SEQUENCE LISTING (177 sequences)

SEQ ID NO: 1 - protein, 736 aa, unidentified
  source 1..736: note = Dependoparvovirus Adeno-associated dependoparvovirus A
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 2 - protein, 736 aa, synthetic construct
  REGION 1..736: note = CAM640-01 capsid
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSHAPRVEELLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 3 - DNA, 2211 bp, synthetic construct
  misc_feature 1..2211: note = CAM640-01 capsid
  source 1..2211: mol_type = other DNA
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtcccacgccccccgggtggaggagttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaa

SEQ ID NO: 4 - protein, 736 aa, synthetic construct
  REGION 1..736: note = CAM640-02 capsid
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSMAPRQEGLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 5 - DNA, 2211 bp, synthetic construct
  misc_feature 1..2211: note = CAM640-02 capsid
  source 1..2211: mol_type = other DNA
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccatggccccccggcaggagggcttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaa

SEQ ID NO: 6 - protein, 736 aa, synthetic construct
  REGION 1..736: note = CAM640-03 capsid
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSHTTDCANLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 7 - DNA, 2211 bp, synthetic construct
  misc_feature 1..2211: note = CAM640-03 capsid
  source 1..2211: mol_type = other DNA
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtcccacaccaccgactgcgccaacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaa

SEQ ID NO: 8 - protein, 736 aa, synthetic construct
  REGION 1..736: note = CAM640-04 capsid
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSAAPRSETLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 9 - DNA, 2211 bp, synthetic construct
  misc_feature 1..2211: note = CAM640-04 capsid
  source 1..2211: mol_type = other DNA
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccgccgccccccggtccgagaccttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaa

SEQ ID NO: 10 - protein, 736 aa, synthetic construct
  REGION 1..736: note = CAM640-05 capsid
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSQAPRQEGLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 11 - DNA, 2211 bp, synthetic construct
  misc_feature 1..2211: note = CAM640-05 capsid
  source 1..2211: mol_type = other DNA
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtcccaggccccccggcaggagggcttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaa

SEQ ID NO: 12 - protein, 736 aa, synthetic construct
  REGION 1..736: note = CAM640-06 capsid
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSVAPRDPALLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 13 - DNA, 2211 bp, synthetic construct
  misc_feature 1..2211: note = CAM640-06 capsid
  source 1..2211: mol_type = other DNA
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccgtggccccccgggaccccgccttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaa

SEQ ID NO: 14 - protein, 736 aa, synthetic construct
  REGION 1..736: note = CAM640-07 capsid
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSSAPRSENLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 15 - DNA, 2211 bp, synthetic construct
  misc_feature 1..2211: note = CAM640-07 capsid
  source 1..2211: mol_type = other DNA
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccagcgccccccggagcgagaacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaa

SEQ ID NO: 16 - skipped

SEQ ID NO: 17 - protein, 7 aa, synthetic construct
  REGION 1..7: note = 454-460 region of CAM640-01
HAPRVEE

SEQ ID NO: 18 - protein, 7 aa, synthetic construct
  REGION 1..7: note = 454-460 region of CAM640-02
MAPRQEG

SEQ ID NO: 19 - protein, 7 aa, synthetic construct
  REGION 1..7: note = 454-460 region of CAM640-03
HTTDCAN

SEQ ID NO: 20 - protein, 7 aa, synthetic construct
  REGION 1..7: note = 454-460 region of CAM640-04
AAPRSET

SEQ ID NO: 21 - protein, 7 aa, synthetic construct
  REGION 1..7: note = 454-460 region of CAM640-05
QAPRQEG

SEQ ID NO: 22 - protein, 7 aa, synthetic construct
  REGION 1..7: note = 454-460 region of CAM640-06
VAPRDPA

SEQ ID NO: 23 - protein, 7 aa, synthetic construct
  REGION 1..7: note = 454-460 region of CAM640-07
SAPRSEN

SEQ ID NO: 24 - skipped

SEQ ID NO: 25 - protein, 736 aa, unidentified
  source 1..736: note = Dependoparvovirus Adeno-associated dependoparvovirus A
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 26 - protein, 735 aa, unidentified [sequence omitted]

SEQ ID NO: 27 - protein, 736 aa, unidentified
  source 1..736: note = Dependoparvovirus Adeno-associated dependoparvovirus A
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 28 - protein, 734 aa, unidentified
  source 1..734: note = Dependoparvovirus Adeno-associated dependoparvovirus A
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 29 - protein, 724 aa, unidentified
  source 1..724: note = Dependoparvovirus Adeno-associated dependoparvovirus A
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 30 - protein, 737 aa, unidentified
  source 1..737: note = Dependoparvovirus Adeno-associated dependoparvovirus A
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 31 - protein, 738 aa, unidentified
  source 1..738: note = Dependoparvovirus Adeno-associated dependoparvovirus A
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 32 - protein, 736 aa, unidentified
  source 1..736: note = Dependoparvovirus Adeno-associated dependoparvovirus A
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 33 - protein, 738 aa, unidentified
  source 1..738: note = Dependoparvovirus Adeno-associated dependoparvovirus A
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 34 - protein, 733 aa, unidentified
  source 1..733: note = Dependoparvovirus Adeno-associated dependoparvovirus A
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPLESPQEPDSSSGIGKKGKQPARKRLNFEEDTGAGDGPPEGSDTSAMSSDIEMRAAPGGNAVDAGQGSDGVGNASGDWHCDSTWSEGKVTTTSTRTWVLPTYNNHLYLRLGTTSSSNTYNGFSTPWGYFDFNRFHCHFSPRDWQRLINNNWGLRPKAMRVKIFNIQVKEVTTSNGETTVANNLTSTVQIFADSSYELPYVMDAGQEGSLPPFPNDVFMVPQYGYCGIVTGENQNQTDRNAFYCLEYFPSQMLRTGNNFEMAYNFEKVPFHSMYAHSQSLDRLMNPLLDQYLWHLQSTTSGETLNQGNAATTFGKIRSGDFAFYRKNWLPGPCVKQQRFSKTASQNYKIPASGGNALLKYDTHYTLNNRWSNIAPGPPMATAGPSDGDFSNAQLIFPGPSVTGNTTTSANNLLFTSEEEIAATNPRDTDMFGQIADNNQNATTAPITGNVTAMGVLPGMVWQNRDIYYQGPIWAKIPHADGHFHPSPLIGGFGLKHPPPQIFIKNTPVPANPATTFTAARVDSFITQYSTGQVAVQIEWEIEKERSKRWNPEVQFTSNYGNQSSMLWAPDTTGKYTEPRVIGSRYLTNHL

SEQ ID NO: 35 - protein, 7 aa, synthetic construct
  REGION 1..7: note = Targeting peptide sequence
  VARIANT 7: note = X can be G or S
NSVRDLX

SEQ ID NO: 36 - protein, 7 aa, synthetic construct
  REGION 1..7: note = Targeting peptide sequence
PRSVTVP

SEQ ID NO: 37 - protein, 7 aa, synthetic construct
  REGION 1..7: note = Targeting peptide sequence
  VARIANT 6: note = X can be any amino acid
  VARIANT 7: note = X can be S or A
NSVSSXX

SEQ ID NO: 38 - protein, 6 aa, synthetic construct
  REGION 1..6: note = Targeting peptide sequence
NGRAHA

SEQ ID NO: 39 - protein, 7 aa, synthetic construct
  REGION 1..7: note = Targeting peptide sequence
QPEHSST

SEQ ID NO: 40 - protein, 7 aa, synthetic construct
  REGION 1..7: note = Targeting peptide sequence
VNTANST

SEQ ID NO: 41 - protein, 6 aa, synthetic construct
  REGION 1..6: note = Targeting peptide sequence
HGPMQS

SEQ ID NO: 42 - protein, 7 aa, synthetic construct
  REGION 1..7: note = Targeting peptide sequence
PHKPPLA

SEQ ID NO: 43 - protein, 7 aa, synthetic construct
  REGION 1..7: note = Targeting peptide sequence
IKNNEMW

SEQ ID NO: 44 - protein, 7 aa, synthetic construct
  REGION 1..7: note = Targeting peptide sequence
RNLDTPM

SEQ ID NO: 45 - protein, 7 aa, synthetic construct
  REGION 1..7: note = Targeting peptide sequence
VDSHRQS

SEQ ID NO: 46 - protein, 7 aa, synthetic construct
  REGION 1..7: note = Targeting peptide sequence
YDSKTKT

SEQ ID NO: 47 - protein, 7 aa, synthetic construct
  REGION 1..7: note = Targeting peptide sequence
SQLPHQK

SEQ ID NO: 48 - protein, 7 aa, synthetic construct
  REGION 1..7: note = Targeting peptide sequence
STMQQNT

SEQ ID NO: 49 - protein, 7 aa, synthetic construct
  REGION 1..7: note = Targeting peptide sequence
TERYMTQ

SEQ ID NO: 50 - protein, 7 aa, synthetic construct
  REGION 1..7: note = Targeting peptide sequence
QPEHSST

SEQ ID NO: 51 - protein, 7 aa, synthetic construct
  REGION 1..7: note = Targeting peptide sequence
DASLSTS

SEQ ID NO: 52 - protein, 6 aa, synthetic construct
  REGION 1..6: note = Targeting peptide sequence
DLPNKT

SEQ ID NO: 53 - protein, 7 aa, synthetic construct
  REGION 1..7: note = Targeting peptide sequence
DLTAARL

SEQ ID NO: 54 - protein, 7 aa, synthetic construct
  REGION 1..7: note = Targeting peptide sequence
EPHQFNY

SEQ ID NO: 55 - protein, 7 aa, synthetic construct
  REGION 1..7: note = Targeting peptide sequence
EPQSNHT

SEQ ID NO: 56 - protein, 7 aa, synthetic construct
  REGION 1..7: note = Targeting peptide sequence
MSSWPSQ

SEQ ID NO: 57 - protein, 7 aa, synthetic construct
  REGION 1..7: note = Targeting peptide sequence
NPKHNAT

SEQ ID NO: 58 - protein, 7 aa, synthetic construct
  REGION 1..7: note = Targeting peptide sequence
PDGMRTT

SEQ ID NO: 59 - protein, 7 aa, synthetic construct
  REGION 1..7: note = Targeting peptide sequence
PNNNKTT

SEQ ID NO: 60 - protein, 7 aa, synthetic construct
  REGION 1..7: note = Targeting peptide sequence
QSTTHDS

SEQ ID NO: 61 - protein, 7 aa, synthetic construct
  REGION 1..7: note = Targeting peptide sequence
TGSKQKQ

SEQ ID NO: 62 - protein, 7 aa, synthetic construct
  REGION 1..7: note = Targeting peptide sequence
SLKHQAL

SEQ ID NO: 63 - protein, 7 aa, synthetic construct
  REGION 1..7: note = Targeting peptide sequence
SPIDGEQ

SEQ ID NO: 64 - protein, 8 aa, synthetic construct
  REGION 1..8: note = Targeting peptide sequence
WIFPWIQL

SEQ ID NO: 65 - protein, 9 aa, synthetic construct
  REGION 1..9: note = Targeting peptide sequence
CDCRGDCFC

SEQ ID NO: 66 - protein, 5 aa, synthetic construct
  REGION 1..5: note = Targeting peptide sequence
CNGRC

SEQ ID NO: 67 - protein, 7 aa, synthetic construct
  REGION 1..7: note = Targeting peptide sequence
CPRECES

SEQ ID NO: 68 - protein, 10 aa, synthetic construct
  REGION 1..10: note = Targeting peptide sequence
CTTHWGFTLC

SEQ ID NO: 69 - protein, 9 aa, synthetic construct
  REGION 1..9: note = Targeting peptide sequence
CGRRAGGSC

SEQ ID NO: 70 - protein, 9 aa, synthetic construct
  REGION 1..9: note = Targeting peptide sequence
CKGGRAKDC

SEQ ID NO: 71 - protein, 9 aa, synthetic construct
  REGION 1..9: note = Targeting peptide sequence
CVPELGHEC

SEQ ID NO: 72 - protein, 9 aa, synthetic construct
  REGION 1..9: note = Targeting peptide sequence
CRRETAWAK

SEQ ID NO: 73 - protein, 14 aa, synthetic construct
  REGION 1..14: note = Targeting peptide sequence
VSWFSHRYSPFAVS

SEQ ID NO: 74 - protein, 13 aa, synthetic construct
  REGION 1..13: note = Targeting peptide sequence
GYRDGYAGPILYN

SEQ ID NO: 75-76 - skipped (2 entries)

SEQ ID NO: 77 - protein, 7 aa, synthetic construct
  REGION 1..7: note = Targeting peptide sequence
RPLPPLP

SEQ ID NO: 78 - protein, 7 aa, synthetic construct
  REGION 1..7: note = Targeting peptide sequence
APPLPPR

SEQ ID NO: 79 - protein, 12 aa, synthetic construct
  REGION 1..12: note = Targeting peptide sequence
DVFYPYPYASGS

SEQ ID NO: 80 - protein, 6 aa, synthetic construct
  REGION 1..6: note = Targeting peptide sequence
MYWYPY

SEQ ID NO: 81 - protein, 12 aa, synthetic construct
  REGION 1..12: note = Targeting peptide sequence
DITWDQLWDLMK

SEQ ID NO: 82 - protein, 8 aa, synthetic construct
  REGION 1..8: note = Targeting peptide sequence
  VARIANT 5: note = X can be G or L
CWDDXWLC

SEQ ID NO: 83 - protein, 14 aa, synthetic construct
  REGION 1..14: note = Targeting peptide sequence
EWCEYLGGYLRCYA

SEQ ID NO: 84 - protein, 14 aa, synthetic construct
  REGION 1..14: note = Targeting peptide sequence
  VARIANT 2: note = X can be any amino acid
  VARIANT 4..5: note = X can be any amino acid
  VARIANT 8: note = X can be any amino acid
  VARIANT 11: note = X can be any amino acid
  VARIANT 13: note = X can be any amino acid
YXCXXGPXTWXCXP

SEQ ID NO: 85 - protein, 14 aa, synthetic construct
  REGION 1..14: note = Targeting peptide sequence
IEGPTLRQWLAARA

SEQ ID NO: 86 - skipped

SEQ ID NO: 87 - protein, 7 aa, synthetic construct
  REGION 1..7: note = Targeting peptide sequence
  VARIANT 1: note = X can be any amino acid
  VARIANT 3..4: note = X can be any amino acid
XFXXYLW

SEQ ID NO: 88 - protein, 6 aa, synthetic construct
  REGION 1..6: note = Targeting peptide sequence
RWGLCD

SEQ ID NO: 89 - protein, 13 aa, synthetic construct
  REGION 1..13: note = Targeting peptide sequence
MSRPACPPNDKYE

SEQ ID NO: 90 - protein, 8 aa, synthetic construct
  REGION 1..8: note = Targeting peptide sequence
CLRSGRGC

SEQ ID NO: 91 - protein, 9 aa, synthetic construct
  REGION 1..9: note = Targeting peptide sequence
CHWMFSPWC

SEQ ID NO: 92 - skipped

SEQ ID NO: 93 - protein, 8 aa, synthetic construct
  REGION 1..8: note = Targeting peptide sequence
CSSRLDAC

SEQ ID NO: 94 - protein, 7 aa, synthetic construct
  REGION 1..7: note = Targeting peptide sequence
CLPVASC

SEQ ID NO: 95 - protein, 13 aa, synthetic construct
  REGION 1..13: note = Targeting peptide sequence
CGFECVRQCPERC

SEQ ID NO: 96 - protein, 13 aa, synthetic construct
  REGION 1..13: note = Targeting peptide sequence
CVALCREACGEGC

SEQ ID NO: 97 - protein, 9 aa, synthetic construct
  REGION 1..9: note = Targeting peptide sequence
SWCEPGWCR

SEQ ID NO: 98 - protein, 6 aa, synthetic construct
  REGION 1..6: note = Targeting peptide sequence
YSGWGW

SEQ ID NO: 99 - protein, 7 aa, synthetic construct
  REGION 1..7: note = Targeting peptide sequence
GLSGGRS

SEQ ID NO: 100 - protein, 7 aa, synthetic construct
  REGION 1..7: note = Targeting peptide sequence
LMLPRAD

SEQ ID NO: 101 - protein, 9 aa, synthetic construct
  REGION 1..9: note = Targeting peptide sequence
CSCFRDVCC

SEQ ID NO: 102 - protein, 9 aa, synthetic construct
  REGION 1..9: note = Targeting peptide sequence
CRDVVSVIC

SEQ ID NO: 103 - protein, 5 aa, synthetic construct
  REGION 1..5: note = Targeting peptide sequence
CNGRC

SEQ ID NO: 104 - protein, 6 aa, synthetic construct
  REGION 1..6: note = Targeting peptide sequence
MARSGL

SEQ ID NO: 105 - protein, 6 aa, synthetic construct
  REGION 1..6: note = Targeting peptide sequence
MARAKE

SEQ ID NO: 106 - protein, 6 aa, synthetic construct
  REGION 1..6: note = Targeting peptide sequence
MSRTMS

SEQ ID NO: 107 - protein, 6 aa, synthetic construct
  REGION 1..6: note = Targeting peptide sequence
KCCYSL

SEQ ID NO: 108 - protein, 14 aa, synthetic construct
  REGION 1..14: note = Targeting peptide sequence
MYWGDSHWLQYWYE

SEQ ID NO: 109 - protein, 7 aa, synthetic construct
  REGION 1..7: note = Targeting peptide sequence
MQLPLAT

SEQ ID NO: 110 - protein, 4 aa, synthetic construct
  REGION 1..4: note = Targeting peptide sequence
EWLS

SEQ ID NO: 111 - protein, 4 aa, synthetic construct
  REGION 1..4: note = Targeting peptide sequence
SNEW

SEQ ID NO: 112 - protein, 4 aa, synthetic construct
  REGION 1..4: note = Targeting peptide sequence
TNYL

SEQ ID NO: 113 - protein, 8 aa, synthetic construct
  REGION 1..8: note = Targeting peptide sequence
WIFPWIQL

SEQ ID NO: 114 - protein, 12 aa, synthetic construct
  REGION 1..12: note = Targeting peptide sequence
WDLAWMFRLPVG

SEQ ID NO: 115 - protein, 13 aa, synthetic construct
  REGION 1..13: note = Targeting peptide sequence
CTVALPGGYVRVC

SEQ ID NO: 116 - protein, 9 aa, synthetic construct
  REGION 1..9: note = Targeting peptide sequence
CVPELGHEC

SEQ ID NO: 117 - protein, 9 aa, synthetic construct
  REGION 1..9: note = Targeting peptide sequence
CGRRAGGSC

SEQ ID NO: 118 - protein, 13 aa, synthetic construct
  REGION 1..13: note = Targeting peptide sequence
CVAYCIEHHCWTC

SEQ ID NO: 119 - protein, 12 aa, synthetic construct
  REGION 1..12: note = Targeting peptide sequence
CVFAHNYDYLVC

SEQ ID NO: 120 - protein, 10 aa, synthetic construct
  REGION 1..10: note = Targeting peptide sequence
CVFTSNYAFC

SEQ ID NO: 121 - protein, 7 aa, synthetic construct
  REGION 1..7: note = Targeting peptide sequence
VHSPNKK

SEQ ID NO: 122 - protein, 9 aa, synthetic construct
  REGION 1..9: note = Targeting peptide sequence
CDCRGDCFC

SEQ ID NO: 123 - protein, 7 aa, synthetic construct
  REGION 1..7: note = Targeting peptide sequence
CRGDGWC

SEQ ID NO: 124 - protein, 6 aa, synthetic construct
  REGION 1..6: note = Targeting peptide sequence
  VARIANT 1: note = X can be any amino acid
  VARIANT 6: note = X can be any amino acid
XRGCDX

SEQ ID NO: 125 - skipped

SEQ ID NO: 126 - protein, 10 aa, synthetic construct
  REGION 1..10: note = Targeting peptide sequence
CTTHWGFTLC

SEQ ID NO: 127 - protein, 11 aa, synthetic construct
  REGION 1..11: note = Targeting peptide sequence
SGKGPRQITAL

SEQ ID NO: 128 - skipped

SEQ ID NO: 129 - protein, 6 aa, synthetic construct
  REGION 1..6: note = Targeting peptide sequence
VYMSPF

SEQ ID NO: 130 - protein, 7 aa, synthetic construct
  REGION 1..7: note = Targeting peptide sequence
MQLPLAT

SEQ ID NO: 131 - protein, 7 aa, synthetic construct
  REGION 1..7: note = Targeting peptide sequence
ATWLPPR

SEQ ID NO: 132 - protein, 12 aa, synthetic construct
  REGION 1..12: note = Targeting peptide sequence
HTMYYHHYQHHL

SEQ ID NO: 133 - protein, 19 aa, synthetic construct
  REGION 1..19: note = Targeting peptide sequence
SEVGCRAGPLQWLCEKYFG

SEQ ID NO: 134 - protein, 18 aa, synthetic construct
  REGION 1..18: note = Targeting peptide sequence
CGLLPVGRPDRNVWRWLC

SEQ ID NO: 135 - protein, 15 aa, synthetic construct
  REGION 1..15: note = Targeting peptide sequence
CKGQCDRFKGLPWEC

SEQ ID NO: 136 - protein, 5 aa, synthetic construct
  REGION 1..5: note = Targeting peptide sequence
SGRSA

SEQ ID NO: 137 - protein, 4 aa, synthetic construct
  REGION 1..4: note = Targeting peptide sequence
WGFP

SEQ ID NO: 138 - protein, 6 aa, synthetic construct
  REGION 1..6: note = Targeting peptide sequence
  VARIANT 3..4: note = X can be any amino acid
LWXXAR

SEQ ID NO: 139 - protein, 7 aa, synthetic construct
  REGION 1..7: note = Targeting peptide sequence
  VARIANT 1: note = X can be any amino acid
  VARIANT 3..4: note = X can be any amino acid
XFXXYLW

SEQ ID NO: 140 - protein, 17 aa, synthetic construct
  REGION 1..17: note = Targeting peptide sequence
AEPMPHSLNFSQYLWYT

SEQ ID NO: 141 - protein, 6 aa, synthetic construct
  REGION 1..6: note = Targeting peptide sequence
  VARIANT 4: note = X can be W or F
WAYXSP

SEQ ID NO: 142 - protein, 7 aa, synthetic construct
  REGION 1..7: note = Targeting peptide sequence
IELLQAR

SEQ ID NO: 143 - protein, 12 aa, synthetic construct
  REGION 1..12: note = Targeting peptide sequence
DITWDQLWDLMK

SEQ ID NO: 144 - protein, 16 aa, synthetic construct
  REGION 1..16: note = Targeting peptide sequence
AYTKCSRQWRTCMTTH

SEQ ID NO: 145 - protein, 15 aa, synthetic construct
  REGION 1..15: note = Targeting peptide sequence
PQNSKIPGPTFLDPH

SEQ ID NO: 146 - protein, 15 aa, synthetic construct
  REGION 1..15: note = Targeting peptide sequence
SMEPALPDWWWKMFK

SEQ ID NO: 147 - protein, 16 aa, synthetic construct
  REGION 1..16: note = Targeting peptide sequence
ANTPCGPYTHDCPVKR

SEQ ID NO: 148 - protein, 12 aa, synthetic construct
  REGION 1..12: note = Targeting peptide sequence
TACHQHVRMVRP

SEQ ID NO: 149 - protein, 12 aa, synthetic construct
  REGION 1..12: note = Targeting peptide sequence
VPWMEPAYQRFL

SEQ ID NO: 150 - protein, 8 aa, synthetic construct
  REGION 1..8: note = Targeting peptide sequence
DPRATPGS

SEQ ID NO: 151 - protein, 12 aa, synthetic construct
  REGION 1..12: note = Targeting peptide sequence
FRPNRAQDYNTN

SEQ ID NO: 152 - protein, 9 aa, synthetic construct
  REGION 1..9: note = Targeting peptide sequence
CTKNSYLMC

SEQ ID NO: 153 - protein, 11 aa, synthetic construct
  REGION 1..11: note = Targeting peptide sequence
  VARIANT 2: note = X can be R or Q
  VARIANT 3: note = X can be L or R
  VARIANT 5: note = X can be G or N
  VARIANT 6..7: note = X can be any amino acid
  VARIANT 9: note = X can be A or V
CXXTXXXGXGC

SEQ ID NO: 154 - protein, 9 aa, synthetic construct
  REGION 1..9: note = Targeting peptide sequence
CPIEDRPMC

SEQ ID NO: 155 - protein, 12 aa, synthetic construct
  REGION 1..12: note = Targeting peptide sequence
HEWSYLAPYPWF

SEQ ID NO: 156 - protein, 9 aa, synthetic construct
  REGION 1..9: note = Targeting peptide sequence
MCPKHPLGC

SEQ ID NO: 157 - protein, 15 aa, synthetic construct
  REGION 1..15: note = Targeting peptide sequence
RMWPSSTVNLSAGRR

SEQ ID NO: 158 - protein, 20 aa, synthetic construct
  REGION 1..20: note = Targeting peptide sequence
SAKTAVSQRVWLPSHRGGEP

SEQ ID NO: 159 - protein, 20 aa, synthetic construct
  REGION 1..20: note = Targeting peptide sequence
KSREHVNNSACPSKRITAAL

SEQ ID NO: 160 - protein, 4 aa, synthetic construct
  REGION 1..4: note = Targeting peptide sequence
EGFR

SEQ ID NO: 161 - protein, 6 aa, synthetic construct
  REGION 1..6: note = Targeting peptide sequence
AGLGVR

SEQ ID NO: 162 - protein, 15 aa, synthetic construct
  REGION 1..15: note = Targeting peptide sequence
GTRQGHTMRLGVSDG

SEQ ID NO: 163 - protein, 15 aa, synthetic construct
  REGION 1..15: note = Targeting peptide sequence
IAGLATPGWSHWLAL

SEQ ID NO: 164 - protein, 7 aa, synthetic construct
  REGION 1..7: note = Targeting peptide sequence
SMSIARL

SEQ ID NO: 165 - protein, 7 aa, synthetic construct
  REGION 1..7: note = Targeting peptide sequence
HTFEPGV

SEQ ID NO: 166 - protein, 14 aa, synthetic construct
  REGION 1..14: note = Targeting peptide sequence
NTSLKRISNKRRRK

SEQ ID NO: 167 - protein, 15 aa, synthetic construct
  REGION 1..15: note = Targeting peptide sequence
LRIKRKRRKRKKTRK

SEQ ID NO: 168 - protein, 4 aa, synthetic construct
RGNR

SEQ ID NO: 169 - protein, 5 aa, synthetic construct
FVFLP

SEQ ID NO: 170 - protein, 9 aa, synthetic construct
SGSAQNKDL

SEQ ID NO: 171 - protein, 9 aa, synthetic construct
SHAPRVEEL

SEQ ID NO: 172 - protein, 9 aa, synthetic construct
SMAPRQEGL

SEQ ID NO: 173 - protein, 9 aa, synthetic construct
SHTTDCANL

SEQ ID NO: 174 - protein, 9 aa, synthetic construct
SAAPRSETL

SEQ ID NO: 175 - protein, 9 aa, synthetic construct
SQAPRQEGL

SEQ ID NO: 176 - protein, 9 aa, synthetic construct
SVAPRDPAL

SEQ ID NO: 177 - protein, 9 aa, synthetic construct
SSAPRSENL

(19) United States
(12) Patent Application Publication
(10) Pub. No.: US 2024/0123085 A1
(43) Pub. Date: Apr. 18, 2024

(54) AAV VECTORS TARGETING T-CELLS

(71) Applicant: Ginkgo Bioworks, Inc., Boston, MA (US)

(72) Inventor: James Kennon Smith, Durham, NC (US)

(21) Appl. No.: 18/221,211

(22) Filed: Jul. 12, 2023

Related U.S. Application Data
(63) Continuation of application No. PCT/US22/12542, filed on Jan. 14, 2022.
(60) Provisional application No. 63/137,497, filed on Jan. 14, 2021.

(51) Int. Cl. A61K 48/00; A61K 45/06; C07K 14/005; C12N 5/0783; C12N 15/86

(52) U.S. Cl. CPC A61K 48/005; A61K 45/06; C07K 14/005; C12N 5/0636; C12N 15/86; C12N 2750/14122; C12N 2750/14143

(57) ABSTRACT

The disclosure provides variant AAV capsid proteins and AAV capsids and virus vectors comprising the same. The virus vectors described herein may have increased transduction in a target cell of interest, such as a T-cell, compared to native AAV capsid sequences. The disclosure also provides methods of administering the virus vectors and virus capsids of the disclosure to a cell or to a patient in need thereof.

### CROSS REFERENCE TO RELATED APPLICATIONS

The present Application claims the benefit of priority to U.S. Provisional Application No. 63/137,497, filed on Jan. 14, 2021, the contents of which are hereby incorporated by reference in its entirety for all purposes.

### FIELD OF THE DISCLOSURE

The present disclosure relates to variant capsid proteins from adeno-associated virus (AAV) and virus capsids and virus vectors comprising the same. In particular, the disclosure relates to variant AAV capsid proteins and AAV capsids comprising the same that can be incorporated into virus vectors to confer a phenotype of enhanced cellular transduction of T-cells in vivo and/or ex vivo.

### DESCRIPTION OF THE TEXT FILE SUBMITTED ELECTRONICALLY

The contents of the text file submitted electronically herewith are incorporated by reference in their entirety: a computer readable format copy of the Sequence Listing (filename: STRD_022_01WO_Sequence_Listing.txt, date recorded Jan. 13, 2022, file size ˜163.4 kilobytes).

### BACKGROUND

Adeno-associated viruses (AAV) are small, single-stranded DNA viruses that belong to the genus Dependovirus, of the Parvoviridae family. AAVs are promising viral vectors for gene therapy due to their ability to infect numerous cell and tissue types, their lack of pathogenicity, their low immunogenicity, and their ability to effectively transduce non-dividing cells. Each of the known AAV serotypes has a differential ability to infect a particular cell type.

There is an interest in using AAVs to target T-cells. For example, AAVs targeting T-cells may be used in gene therapy methods for preventing, limiting, and/or reversing T-cell exhaustion. T-cell exhaustion is a state of T-cell dysfunction that arises during many chronic infections and cancer, and has also been shown to reduce the effectiveness of CAR-T therapies. However, AAVs do not typically transduce T-cells at high levels.

Accordingly, there is a need in the art for improved AAV vectors that can target T-cells with enhanced transduction efficiency.

### SUMMARY

The instant disclosure relates to adeno-associated virus (AAV) capsid proteins comprising one or more transduction-associated peptides, and AAV capsids and viral vectors comprising the same. The disclosed transduction-associated peptides can enhance the cellular transduction of the AAV vectors into desired cell types, such as T-cells.

The disclosure provides recombinant adeno-associated virus (AAV) vectors comprising a capsid protein, wherein the capsid protein comprises a transduction-associated peptide having the sequence of any one of SEQ ID NOs: 17 to 23. In some embodiments, the capsid protein comprises an amino acid sequence that has at least 90%, at least 95%, at least 96%, at least 97%, at least 98%, or at least 99% identity to SEQ ID NO: 1. In some embodiments, the transduction-associated peptide replaces the amino acids corresponding to amino acids 454-460 of SEQ ID NO: 1. In some embodiments, the capsid protein comprises an amino acid sequence selected from the group consisting of SEQ ID NOs: 2, 4, 6, 8, 10, 12, and 14, or a sequence at least 90%, at least 95%, at least 96%, at least 97%, at least 98%, or at least 99% identical thereto.

The disclosure provides recombinant AAV vectors comprising a capsid protein, wherein the capsid protein comprises the sequence of SEQ ID NO: 1, wherein amino acids 454-460 of SEQ ID NO: 1 are replaced by a transduction-associated peptide comprising the sequence X1-X2-X3-X4-X5-X6-X7 (SEQ ID NO: 24). In some embodiments, X1 is not G, X2 is not S, X3 is not A, X4 is not Q, X5 is not N, X6 is not K, and/or X7 is not D. In some embodiments, X1 is H, M, A, Q, V, or S. In some embodiments, X2 is A or T. In some embodiments, X3 is P or T. In some embodiments, X4 is R or D. In some embodiments, X5 is V, Q, C, S, or D. In some embodiments, X6 is E, A, or P. In some embodiments, X7 is E, G, N, T, or A. In some embodiments, X1 is H, X2 is A, X3 is P, X4 is R, X5 is V, X6 is E, and X7 is E. In some embodiments, X1 is M, X2 is A, X3 is P, X4 is R, X5 is Q, X6 is E, and X7 is G. In some embodiments, X1 is H, X2 is T, X3 is T, X4 is D, X5 is C, X6 is A, and X7 is N. In some embodiments, X1 is A, X2 is A, X3 is P, X4 is R, X5 is S, X6 is E, and X7 is T. In some embodiments, X1 is Q, X2 is A, X3 is P, X4 is R, X5 is Q, X6 is E, and X7 is G. In some embodiments, X1 is V, X2 is A, X3 is P, X4 is R, X5 is D, X6 is P, and X7 is A. In some embodiments, X1 is S, X2 is A, X3 is P, X4 is R, X5 is S, X46 is E, and X7 is N.

In some embodiments, the capsid protein comprises an amino acid sequence having at least about 95%, at least about 96%, at least about 97%, at least about 98%, or at least about 99% identity to SEQ ID NO: 1. In some embodiments, the capsid protein comprises an amino acid sequence having about 99% identity to SEQ ID NO: 1. In some embodiments, the capsid protein comprises an amino acid sequence selected from the group consisting of SEQ ID NOs: 2, 4, 6, 8, 10, 12, and 14.

The disclosure provides recombinant AAV vectors comprising a capsid protein, wherein the capsid protein comprises a transduction-associated peptide having an amino acid sequence of SEQ ID NO: 16, wherein the transduction-associated peptide replaces amino acids 454-460 relative to SEQ ID NO: 1. In some embodiments, the transduction-associated peptide has an amino acid sequence of any one of SEQ ID NOs: 17-23.

The disclosure provides nucleic acids encoding a recombinant AAV capsid protein having the sequence of any one of SEQ ID NOs: 2, 4, 6, 8, 10, 12, and 14. In some embodiments, the nucleic acid comprises a sequence selected from the group consisting of SEQ ID NOs: 3, 5, 7, 9, 11, 13, and 15. In some embodiments, the nucleic acid is a DNA sequence. In some embodiments, the nucleic acid is an RNA sequence. The disclosure provides expression vectors comprising any one of the nucleic acids disclosed herein. The disclosure further provides cells comprising any one of the nucleic acids disclosed herein, or any one of the expression vectors disclosed herein.

In some embodiments, any one of the recombinant AAV vectors disclosed herein further comprise a cargo nucleic acid encapsidated by the capsid protein. In some embodiments, the cargo nucleic acid encodes a therapeutic protein or a therapeutic RNA. In some embodiments, the AAV vector exhibits increased transduction into a cell compared to an AAV vector that does not comprise the transduction-associated peptide. In some embodiments, the cell is a T-cell. In some embodiments, the AAV vector exhibits increased transduction into the nucleus of a T-cell as compared to an AAV vector that does not comprise the transduction-associated peptide. In some embodiments, the AAV vector exhibits increased transduction into the cytosol of a T-cell as compared to an AAV vector that does not comprise the transduction-associated peptide.

The disclosure provides compositions, comprising any one of the recombinant AAV vectors disclosed herein, any one of the nucleic acids disclosed herein, any one of the expression vectors disclosed herein, or any one of the cells disclosed herein. The disclosure further provides pharmaceutical compositions, comprising any one of the cells disclosed herein or any one of the recombinant AAV vectors disclosed herein; and a pharmaceutically acceptable carrier.

The disclosure provides methods of delivering an AAV vector into a cell, comprising contacting the cell with any one of the AAV vectors disclosed herein. In some embodiments, the contacting of the cell is performed in vitro, ex vivo or in vivo. In some embodiments, the cell is a T-cell. The disclosure provides methods of treating a subject in need thereof, comprising administering to the subject an effective amount of any one of the AAV vectors disclosed herein. The disclosure provides methods of treating a subject in need thereof, comprising administering to the subject a cell that has been contacted ex vivo with any one of the AAV vectors disclosed herein. In some embodiments, the subject is a mammal. In some embodiments, the subject is a human. The disclosure provides any one of the AAV vectors disclosed herein for use as a medicament. The disclosure also provides any one of the AAV vectors disclosed herein for use in a method of treatment of a subject in need thereof.

These and other embodiments are addressed in more detail in the detailed description set forth below.

### BRIEF DESCRIPTION OF THE DRAWINGS

FIG. 1 shows the total vector genome (vg) volumetric yield obtained using the manufacturing process described in Example 2 for various AAV vectors comprising variant capsids, as compared to wild type AAV6.

FIG. 2 shows images from a microscopic analysis of T-cells transduced with either wild type AAV6 or AAV vectors comprising the indicated AAV6 capsid variants. Each AAV vector packaged a GFP transgene. Images were obtained after transduction of cells with the AAV vectors using different multiplicities of infection (MOI), as indicated.

FIGS. 3A-3C shows results from a flow cytometry analysis of T-cells transduced with either wild type AAV6 or the indicated AAVs comprising variant capsids, each packaging a GFP transgene. FIG. 3A shows size and granularity (i.e., forward scatter and side scatter) of the tested cell samples, from which the cell population of interest (encircled on the diagram) was identified. FIG. 3B shows size and granularity for only the cell population that was selected for analysis. FIG. 3C shows the fluorescence (FITC) signal measured for the cell population of interest. There was an increase in fluorescence in cells transduced with an AAV vector comprising a STRD-207 capsid, as compared to cells transduced with wild type AAV6.

FIG. 4 shows a plot of the percent GFP positive T-cells obtained from flow cytometry experiments performed with wild type AAV6 or each of the AAVs comprising capsid variants as indicated. The T-cells were derived from two different human donors (Donor 11 and Donor 12). Different MOIs were used, as indicated (10,000, 5,000 and 2,500 for Donor 12 T-cells and 15,000, 7,500 and 3,750 for Donor 11 T-cells).

FIG. 5A and FIG. 5B are bubble plots depicting isolates of individual AAVs comprising variant capsids obtained from the nuclear fraction (FIG. 5A) and the cytosolic fraction (FIG. 5B) of activated T-cells after the three rounds of evolution and selection for T-cell transduction as described in Example 1. Each bubble represents a distinct capsid protein amino acid sequence with the radius of the bubble proportional to the number of reads for that variant in the respective library. The y-axis represents the absolute number of reads. Data are spread along the x-axis for ease of visualization. Dominant isolates were selected for sequencing analysis.

FIG. 6 shows the sequences of the transduction-associated peptides identified in AAV vectors enriched in the nuclear fraction or the cytosolic fraction of T-cells. These transduction-associated peptides were located at amino acids 464-456 of the capsid proteins, wherein the amino acid numbering corresponds to wildtype AAV6 (SEQ ID NO: 1). The sequences shown in FIG. 6 correspond to SEQ ID NOs: 17-23, in order from top to bottom.

### DETAILED DESCRIPTION

Unless otherwise defined, all technical and scientific terms used herein have the same meaning as commonly understood by one of ordinary skill in the art to which this disclosure belongs. The terminology used in the detailed description herein is for the purpose of describing particular embodiments only and is not intended to be limiting.

All publications, patent applications, patents, articles, GenBank or other accession numbers and other references mentioned herein are incorporated by reference in their entireties.

The designation of all amino acid positions in the AAV capsid proteins in the disclosure and the appended claims is with respect to VP1 capsid subunit numbering. It will be understood by those skilled in the art that the modifications described herein if inserted into the AAV cap gene may result in modifications in the VP1, VP2 and/or VP3 capsid subunits. Alternatively, the capsid subunits can be expressed independently to achieve modification in only one or two of the capsid subunits (VP1, VP2, VP3, VP1+VP2, VP1+VP3, or VP2+VP3).

### Definitions

The following terms are used in the description herein and the appended claims:

The singular forms “a,” “an” and “the” are intended to include the plural forms as well, unless the context clearly indicates otherwise.

Furthermore, the term “about” as used herein when referring to a measurable value such as an amount or the length of a polynucleotide or polypeptide sequence, dose, time, temperature, and the like, is meant to encompass variations of±20%, ±10%, ±5%, ±1%, ±0.5%, or even ±0.1% of the specified amount.

Also as used herein, “and/or” refers to and encompasses any and all possible combinations of one or more of the associated listed items, as well as the lack of combinations when interpreted in the alternative (“or”).

Unless the context indicates otherwise, it is specifically intended that the various features described herein can be used in any combination.

Moreover, the present disclosure also contemplates that in some embodiments, any feature or combination of features set forth herein can be excluded or omitted. To illustrate further, if, for example, the specification indicates that a particular amino acid can be selected from A, G, I, L and/or V, this language also indicates that the amino acid can be selected from any subset of these amino acid(s) for example A, G, I or L; A, G, I or V; A or G; only L; etc., as if each such sub-combination is expressly set forth herein. Moreover, such language also indicates that one or more of the specified amino acids can be disclaimed. For example, in some embodiments the amino acid is not A, G or I; is not A; is not G or V; etc., as if each such possible disclaimer is expressly set forth herein.

As used herein, the terms “reduce,” “reduces,” “reduction” and similar terms mean a decrease of at least about 10%, about 15%, about 20%, about 25%, about 35%, about 50%, about 75%, about 80%, about 85%, about 90%, about 95%, about 97% or more.

As used herein, the terms “enhance,” “enhances,” “enhancement” and similar terms indicate an increase of at least about 10%, about 15%, about 20%, about 25%, about 35%, about 50%, about 75%, about 80%, about 85%, about 90%, about 95%, about 100%, about 150%, about 200%, about 300%, about 400%, about 500% or more.

The term “parvovirus” as used herein encompasses the family Parvoviridae, including autonomously replicating parvoviruses and dependoviruses. The autonomous parvoviruses include members of the genera Protoparvovirus, Erythroparvovirus, Bocaparvirus, and Densovirus subfamily. Exemplary autonomous parvoviruses include, but are not limited to, minute virus of mouse, bovine parvovirus, canine parvovirus, chicken parvovirus, feline panleukopenia virus, feline parvovirus, goose parvovirus, H1 parvovirus, muscovy duck parvovirus, B19 virus, and any other autonomous parvovirus now known or later discovered. Other autonomous parvoviruses are known to those skilled in the art. See, e.g., BERNARD N. FIELDS et al, VIROLOGY, volume 2, chapter 69 (4th ed., Lippincott-Raven Publishers; Cotmore et al. Archives of Virology DOI 10.1007/s00705-013-1914-I).The terms “subject,” “individual,” and “patient” are used interchangeably herein to refer to a vertebrate, such as a mammal. The mammal may be, for example, a mouse, a rat, a rabbit, a cat, a dog, a pig, a sheep, a horse, a non-human primate (e.g., cynomolgus monkey, chimpanzee), or a human. A subject's tissues, cells, or derivatives thereof, obtained in vivo or cultured in vitro are also encompassed. A human subject may be an adult, a teenager, a child (2 years to 14 years of age), an infant (1 month to 24 months), or a neonate (up to 1 month). In some embodiments, the adults are seniors about 65 years or older, or about 60 years or older. In some embodiments, the subject is a pregnant woman or a woman intending to become pregnant. In some embodiments, the subject is “in need” of the methods described herein.

As used herein, the term “adeno-associated virus” (AAV), includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, AAV type rh32.33, AAV type rh8, AAV type rh10, AAV type rh74, AAV type hu.68, avian AAV, bovine AAV, canine AAV, equine AAV, ovine AAV, snake AAV, bearded dragon AAV, AAV2i8, AAV2g9, AAV-LK03, AAV7m8, AAV Anc80, AAV PHP.B, and any other AAV now known or later discovered. See, e.g., BERNARD N. FIELDS et al., VIROLOGY, volume 2, chapter 69 (4th ed., Lippincott-Raven Publishers). A number of AAV serotypes and clades have been identified (see, e.g., Gao et al, (2004) J. Virology 78:6381-6388; Moris et al, (2004) Virology 33-:375-383; and Table 2).

As used herein, the term “chimeric AAV” refers to an AAV comprising a capsid protein with regions, domains, and/or individual amino acids that are derived from two or more different serotypes of AAV. In some embodiments, a chimeric AAV comprises a capsid protein comprised of a first region that is derived from a first AAV serotype and a second region that is derived from a second AAV serotype. In some embodiments, a chimeric AAV comprises a capsid protein comprised of a first region that is derived from a first AAV serotype, a second region that is derived from a second AAV serotype, and a third region that is derived from a third AAV serotype. In some embodiments, the chimeric AAV may comprise regions, domains, individual amino acids derived from two or more of AAV1, AAV2, AAV3, AAV4, AAV5, AAV6, AAV7, AAV8, AAV9, AAV10, AAV11, and/or AAV12. For example, the chimeric AAV may include regions, domains, and/or individual amino acids from a first and a second AAV serotype as shown below (Table 1), wherein AAVX+Y indicates a chimeric AAV including sequences derived from AAVX and AAVY.

TABLE 1
Chimeric AAVs

| | Second AAV serotype | | | | | | | |
|---|---|---|---|---|---|---|---|---|
| | | AAV1 | AAV2 | AAV3 | AAV4 | AAV5 | AAV6 | AAV7 |
| First AAV | AAV1 | X | AAV1 + 2 | AAV1 + 3 | AAV1 + 4 | AAV1 + 5 | AAV1 + 6 | AAV1 + 7 |
| serotype | AAV2 | AAV2 + 1 | X | AAV2 + 3 | AAV2 + 4 | AAV2 + 5 | AAV2 + 6 | AAV2 + 7 |
| | AAV3 | AAV3 + 1 | AAV3 + 2 | X | AAV3 + 4 | AAV3 + 5 | AAV3 + 6 | AAV3 + 7 |
| | AAV4 | AAV4 + 1 | AAV4 + 2 | AAV4 + 3 | X | AAV4 + 5 | AAV4 + 6 | AAV4 + 7 |
| | AAV5 | AAV5 + 1 | AAV5 + 2 | AAV5 + 3 | AAV5 + 4 | X | AAV5 + 6 | AAV5 + 7 |
| | AAV6 | AAV6 + 1 | AAV6 + 2 | AAV6 + 3 | AAV6 + 4 | AAV6 + 5 | X | AAV6 + 7 |
| | AAV7 | AAV7 + 1 | AAV7 + 2 | AAV7 + 3 | AAV7 + 4 | AAV7 + 5 | AAV7 + 6 | X |
| | AAV8 | AAV8 + 1 | AAV8 + 2 | AAV8 + 3 | AAV8 + 4 | AAV8 + 5 | AAV8 + 6 | AAV8 + 7 |
| | AAV9 | AAV9 + 1 | AAV9 + 2 | AAV9 + 3 | AAV9 + 4 | AAV9 + 5 | AAV9 + 6 | AAV9 + 7 |
| | AAV10 | AAV10 + 1 | AAV10 + 2 | AAV10 + 3 | AAV10 + 4 | AAV10 + 5 | AAV10 + 6 | AAV10 + 7 |
| | AAV11 | AAV11 + 1 | AAV11 + 2 | AAV11 + 3 | AAV11 + 4 | AAV11 + 5 | AAV11 + 6 | AAV11 + 7 |
| | AAV12 | AAV12 + 1 | AAV12 + 2 | AAV12 + 3 | AAV12 + 4 | AAV12 + 5 | AAV12 + 6 | AAV12 + 7 |

| | Second AAV serotype | | | | | |
|---|---|---|---|---|---|---|
| | | AAV8 | AAV9 | AAV10 | AAV11 | AAV12 |
| First AAV | AAV1 | AAV1 + 8 | AAV1 + 9 | AAV1 + 10 | AAV1 + 11 | AAV1 + 12 |
| serotype | AAV2 | AAV2 + 8 | AAV2 + 9 | AAV2 + 10 | AAV2 + 11 | AAV2 + 12 |
| | AAV3 | AAV3 + 8 | AAV3 + 9 | AAV3 + 10 | AAV3 + 11 | AAV3 + 12 |
| | AAV4 | AAV4 + 8 | AAV4 + 9 | AAV4 + 10 | AAV4 + 11 | AAV4 + 12 |
| | AAV5 | AAV5 + 8 | AAV5 + 9 | AAV5 + 10 | AAV5 + 11 | AAV5 + 12 |
| | AAV6 | AAV6 + 8 | AAV6 + 9 | AAV6 + 10 | AAV6 + 11 | AAV6 + 12 |
| | AAV7 | AAV7 + 8 | AAV7 + 9 | AAV7 + 10 | AAV7 + 11 | AAV7 + 12 |
| | AAV8 | X | AAV8 + 9 | AAV8 + 10 | AAV8 + 11 | AAV8 + 12 |
| | AAV9 | AAV9 + 8 | X | AAV9 + 10 | AAV9 + 11 | AAV9 + 12 |
| | AAV10 | AAV10 + 8 | AAV10 + 9 | X | AAV10 + 11 | AAV10 + 12 |
| | AAV11 | AAV11 + 8 | AAV11 + 9 | AAV11 + 10 | X | AAV11 + 12 |
| | AAV12 | AAV12 + 8 | AAV12 + 9 | AAV12 + 10 | AAV12 + 11 | X |

By including individual amino acids or regions from multiple AAV serotypes in one capsid protein, capsid proteins that have multiple desired properties that are separately derived from the multiple AAV serotypes may be obtained.

The genomic sequences of various serotypes of AAV and the autonomous parvoviruses, as well as the sequences of the native terminal repeats (TRs), Rep proteins, and capsid subunits are known in the art. Such sequences may be found in the literature or in public databases such as GenBank. See, e.g., GenBank Accession Numbers NC_002077, NC_001401, NC_001729, NC_001863, NC_001829, NC_001 862, AAB95450.1, NC_000883, NC_001701, NC_001510, NC_006152, NC_006261, AF063497, U89790, AF043303, AF028705, AF028704, J02275, J01901, J02275, X01457, AF288061, AH009962, AY028226, AY028223, NC_001358, NC_001540, AF513851, AF513852, AY530579; the disclosures of which are incorporated by reference herein for teaching parvovirus and AAV nucleic acid and amino acid sequences. See also, e.g., Srivistava et al., (1983) J. Virology 45:555; Chiorini et al, (1998) J Virology 71:6823; Chiorini et al., (1999) J. Virology 73: 1309; Bantel-Schaal et al., (1999) J Virology 73:939; Xiao et al, (1999) J Virology 73:3994; Muramatsu et al., (1996) Virology 221:208; Shade et al, (1986) J. Virol. 58:921; Gao et al, (2002) Proc. Nat. Acad. Sci. USA 99:11854; Moris et al, (2004) Virology 33:375-383; international patent publications WO 00/28061, WO 99/61601, WO 98/11244; and U.S. Pat. No. 6,156,303; the disclosures of which are incorporated by reference herein for teaching parvovirus and AAV nucleic acid and amino acid sequences. See also Table 2. The capsid structures of autonomous parvoviruses and AAV are described in more detail in BERNARD N. FIELDS et al., VIROLOGY, volume 2, chapters 69 & 70 (4th ed., Lippincott-Raven Publishers). See also, description of the crystal structure of AAV2 (Xie et al., (2002) Proc. Nat. Acad. Sci. 99:10405-10), AAV9 (DiMattia et al., (2012) J. Virol. 86:6947-6958), AAV8 (Nam et al, (2007) J. Virol. 81:12260-12271), AAV6 (Ng et al., (2010) J. Virol. 84:12945-12957), AAV5 (Govindasamy et al. (2013) J. Virol. 87, 11187-11199), AAV4 (Govindasamy et al. (2006) J. Virol. 80:11556-11570), AAV3B (Lerch et al., (2010) Virology 403:26-36), BPV(Kailasan et al., (2015) J. Virol. 89:2603-2614) and CPV (Xie et al, (1996) J. Mol. Biol. 6:497-520 and Tsao et al, (1991) Science 251:1456-64).

TABLE 2

| | GenBank |
|---|---|
| | Accession |
| | Number |
| Complete Genomes | |
| Adeno-associated virus 1 | NC_002077, |
| | AF063497 |
| Adeno-associated virus 2 | NC_001401 |
| Adeno-associated virus 3 | NC_001729 |
| Adeno-associated virus 3B | NC_001863 |
| Adeno-associated virus 4 | NC_001829 |
| Adeno-associated virus 5 | Y18065, |
| | AF085716 |
| Adeno-associated virus 6 | NC_001862, |
| | AAB95450.1 |
| Avian AAV ATCC VR-865 | AY186198, |
| | AY629583, |
| | NC_004828 |
| Avian AAV strain DA-1 | NC_006263, |
| | AY629583 |
| Bovine AAV | NC_005889, |
| | AY388617, |
| | AAR26465 |
| AAV11 | AAT46339, |
| | AY631966 |
| AAV12 | ABI16639, |
| | DQ813647 |
| Clade A | |
| AAV1 | NC_002077, |
| | AF063497 |
| AAV6 | NC_001862 |
| Hu.48 | AY530611 |
| Hu 43 | AY530606 |
| Hu 44 | AY530607 |
| Hu 46 | AY530609 |
| Clade B | |
| Hu. 19 | AY530584 |
| Hu. 20 | AY530586 |
| Hu 23 | AY530589 |
| Hu22 | AY530588 |
| Hu24 | AY530590 |
| Hu21 | AY530587 |
| Hu27 | AY530592 |
| Hu28 | AY530593 |
| Hu 29 | AY530594 |
| Hu63 | AY530624 |
| Hu64 | AY530625 |
| Hu13 | AY530578 |
| Hu56 | AY530618 |
| Hu57 | AY530619 |
| Hu49 | AY530612 |
| Hu58 | AY530620 |
| Hu34 | AY530598 |
| Hu35 | AY530599 |
| AAV2 | NC_001401 |
| Hu45 | AY530608 |
| Hu47 | AY530610 |
| Hu51 | AY530613 |
| Hu52 | AY530614 |
| Hu T41 | AY695378 |
| Hu S17 | AY695376 |
| Hu T88 | AY695375 |
| Hu T71 | AY695374 |
| Hu T70 | AY695373 |
| Hu T40 | AY695372 |
| Hu T32 | AY695371 |
| Hu T17 | AY695370 |
| Hu LG15 | AY695377 |
| Clade C | |
| Hu9 | AY530629 |
| Hu10 | AY530576 |
| Hu11 | AY530577 |
| Hu53 | AY530615 |
| Hu55 | AY530617 |
| Hu54 | AY530616 |
| Hu7 | AY530628 |
| Hu18 | AY530583 |
| Hu15 | AY530580 |
| Hu16 | AY530581 |
| Hu25 | AY530591 |
| Hu60 | AY530622 |
| Ch5 | AY243021 |
| Hu3 | AY530595 |
| Hu1 | AY530575 |
| Hu4 | AY530602 |
| Hu2 | AY530585 |
| Hu61 | AY530623 |
| Clade D | |
| Rh62 | AY530573 |
| Rh48 | AY530561 |
| Rh54 | AY530567 |
| Rh55 | AY530568 |
| Cy2 | AY243020 |
| AAV7 | AF513851 |
| Rh35 | AY243000 |
| Rh37 | AY242998 |
| Rh36 | AY242999 |
| Cy6 | AY243016 |
| Cy4 | AY243018 |
| Cy3 | AY243019 |
| Cy5 | AY243017 |
| Rh13 | AY243013 |
| Clade E | |
| Rh38 | AY530558 |
| Hu66 | AY530626 |
| Hu42 | AY530605 |
| Hu67 | AY530627 |
| Hu40 | AY530603 |
| Hu41 | AY530604 |
| Hu37 | AY530600 |
| Rh40 | AY530559 |
| Rh2 | AY243007 |
| Bb1 | AY243023 |
| Bb2 | AY243022 |
| Rh10 | AY243015 |
| Hu17 | AY530582 |
| Hu6 | AY530621 |
| Rh25 | AY530557 |
| Pi2 | AY530554 |
| Pi1 | AY530553 |
| Pi3 | AY530555 |
| Rh57 | AY530569 |
| Rh50 | AY530563 |
| Rh49 | AY530562 |
| Hu39 | AY530601 |
| Rh58 | AY530570 |
| Rh61 | AY530572 |
| Rh52 | AY530565 |
| Rh53 | AY530566 |
| Rh51 | AY530564 |
| Rh64 | AY530574 |
| Rh43 | AY530560 |
| AAV8 | AF513852 |
| Rh8 | AY242997 |
| Rh1 | AY530556 |
| Clade F | |
| Hu14 (AAV9) | AY530579 |
| Hu31 | AY530596 |
| Hu32 | AY530597 |
| HSC1 | MI332400.1 |
| HSC2 | MI332401.1 |
| HSC3 | MI332402.1 |
| HSC4 | MI332403.1 |
| HSC5 | MI332405.1 |
| HSC6 | MI332404.1 |
| HSC7 | MI332407.1 |
| HSC8 | MI332408.1 |
| HSC9 | MI332409.1 |
| HSC11 | MI332406.1 |
| HSC12 | MI332410.1 |
| HSC13 | MI332411.1 |
| HSC14 | MI332412.1 |
| HSC15 | MI332413.1 |
| HSC16 | MI332414.1 |
| HSC17 | MI332415.1 |
| Hu68 | |
| Clonal Isolate | |
| AAV5 | Y18065, |
| | AF085716 |
| AAV 3 | NC_001729 |
| AAV 3B | NC_001863 |
| AAV4 | NC_001829 |
| Rh34 | AY243001 |
| Rh33 | AY243002 |
| Rh32 | AY243003 |
| Others | |
| Rh74 | |
| Bearded Dragon AAV | |
| Snake AAV | NC_006148.1 |

Recombinant AAV (rAAV) vectors can be produced in culture using viral production cell lines. The terms “viral production cell”, “viral production cell line,” or “viral producer cell” refer to cells used to produce viral vectors. HEK293 and 239T cells are common viral production cell lines. Table 8, below, lists exemplary viral production cell lines for various viral vectors. Production of rAAVs typically requires the presence of three elements in the cells: 1) a transgene flanked by AAV inverted terminal repeat (ITR) sequences, 2) AAV rep and cap genes, and 3) helper virus protein sequences. These three elements may be provided on one or more plasmids, and transfected or transduced into the cells.

TABLE 8
Exemplary viral production cell lines

| Virus Vector | Exemplary Viral Production Cell Line(s) |
|---|---|
| Adenovirus | HEK293, 911, pTG6559, PER.C6, GH329, |
| | N52.E6, HeLa-E1, UR, VLI-293 |
| Adeno-Associated Virus | HEK293, Sf9 |
| (AAV) | |
| Retrovirus | HEK293 |
| Lentivirus | 293T |

As used herein, the term “multiplicity of infection” or “MOI” refers to number of virions contacted with a cell. For example, cultured cells may be contacted with AAVs at an MOI in the range of about 1×10² to about 1×10⁵ virions per cell.

The term “transduction” as used herein refers to a process whereby a nucleic acid (e.g., a transgene) is introduced into a cell by a viral vector. Described herein are modified AAV capsid proteins (e.g., variant capsid proteins) and capsids comprising the same that can be incorporated into virus vectors to confer a phenotype of enhanced cellular transduction in vivo or ex vivo. As used herein, “enhanced transduction,” “enhanced cellular transduction” and similar terms may refer to an increase in transduction from about 1.5-fold to about 100-fold, or more. For example, transduction may be increased by at least 1.5-fold, at least 2-fold, at least 3-fold, at least 4-fold, at least 5-fold, at least 10-fold, at least 20-fold, at least 30-fold, at least 40-fold, at least 50-fold, at least 60-fold, at least 70-fold, at least 80-fold, at least 90-fold, at least 100-fold, or more. Transduction of a modified AAV (e.g., an AAV comprising a capsid variant) may be enhanced relative to a wildtype or native AAV vector. In some embodiments, transduction of an AAV vector comprising a transduction-associated peptide may be enhanced relative to an AAV vector that is otherwise identical but lacks the transduction-associated peptide.

The term “transgene” refers to any nucleic acid sequence used in the transduction of a cell, which can be a cell maintained ex vivo or a cell in an organism. A transgene can be a coding sequence, a non-coding sequence, a cDNA, a gene or fragment or portion thereof, a genomic sequence, a regulatory element and the like. A “transgenic” organism, such as a transgenic plant or transgenic animal, is an organism into which a transgene has been delivered or introduced and the transgene can be expressed in the transgenic organism to produce a product, the presence of which can impart an effect (e.g., a therapeutic or beneficial effect) and/or a phenotype (e.g., a desired or altered phenotype) in the organism.

The term “tropism” as used herein refers to preferential entry of the virus into certain cells or tissues, optionally followed by expression (e.g., transcription and, optionally, translation) of a sequence(s) carried by the viral genome in the cell, e.g., for a recombinant virus, expression of a heterologous nucleic acid(s) of interest.

Those skilled in the art will appreciate that transcription of a heterologous nucleic acid sequence from the viral genome may not be initiated in the absence of trans-acting factors, e.g., for an inducible promoter or otherwise regulated nucleic acid sequence. In the case of a rAAV genome, gene expression from the viral genome may be from a stably integrated provirus, from a non-integrated episome, as well as any other form in which the virus may take within the cell.

As used here, “systemic tropism” and “systemic transduction” (and equivalent terms) indicate that the virus capsid or virus vector of the disclosure exhibits tropism for or transduces, respectively, tissues throughout the body (e.g., brain, lung, skeletal muscle, heart, liver, kidney and/or pancreas). In some embodiments, systemic transduction of muscle tissues (e.g., skeletal muscle, diaphragm and cardiac muscle) is observed. In some embodiments, systemic transduction of skeletal muscle tissues achieved. For example, in some embodiments, essentially all skeletal muscles throughout the body are transduced (although the efficiency of transduction may vary by muscle type). In some embodiments, systemic transduction of limb muscles, cardiac muscle and diaphragm muscle is achieved. Optionally, the virus capsid or virus vector is administered via a systemic route (e.g., systemic route such as intravenously, intra-articularly or intra-lymphatically).

Alternatively, in some embodiments, the capsid or virus vector is delivered locally (e.g., to the footpad, intramuscularly, intradermally, subcutaneously, topically).

Unless indicated otherwise, “efficient transduction” or “efficient tropism,” or similar terms, can be determined by reference to a suitable control (e.g., at least about 50%, about 60%, about 70%, about 80%, about 85%, about 90%, about 95% or more of the transduction or tropism, respectively, of the control). In some embodiments, the virus vector efficiently transduces or has efficient tropism for T-cells, skeletal muscle, cardiac muscle, diaphragm muscle, pancreas (including (3-islet cells), spleen, the gastrointestinal tract (e.g., epithelium and/or smooth muscle), cells of the central nervous system, lung, joint cells, and/or kidney. Suitable controls will depend on a variety of factors including the desired tropism profile. In some embodiments, the suitable control is a wild type or native virus.

Similarly, it can be determined if a virus “does not efficiently transduce” or “does not have efficient tropism” for a target tissue, or similar terms, by reference to a suitable control. In some embodiments, the virus vector does not efficiently transduce (i.e., has does not have efficient tropism) for liver, kidney, gonads and/or germ cells. In some embodiments, undesirable transduction of tissue(s) (e.g., liver) is 20% or less, 10% or less, 5% or less, 1% or less, 0.1% or less of the level of transduction of the desired target tissue(s) (e.g., skeletal muscle, diaphragm muscle, cardiac muscle and/or cells of the central nervous system).

As used herein, the term “polypeptide” encompasses both peptides and proteins, unless indicated otherwise.

A “polynucleotide” is a sequence of nucleotide bases, and may be RNA, DNA or DNA-RNA hybrid sequences (including both naturally occurring and non-naturally occurring nucleotide), but in representative embodiments are either single or double stranded DNA sequences.

As used herein, an “isolated” polynucleotide (e.g., an “isolated DNA” or an “isolated RNA”) means a polynucleotide at least partially separated from at least some of the other components of the naturally occurring organism or virus, for example, the cell or viral structural components or other polypeptides or nucleic acids commonly found associated with the polynucleotide. In representative embodiments an “isolated” nucleotide is enriched by at least about 10-fold, about 100-fold, about 1000-fold, about 10,000-fold or more as compared with the starting material.

Likewise, an “isolated” polypeptide means a polypeptide that is at least partially separated from at least some of the other components of the naturally occurring organism or virus, for example, the cell or viral structural components or other polypeptides or nucleic acids commonly found associated with the polypeptide. In some embodiments, an “isolated” polypeptide is enriched by at least about 10-fold, about 100-fold, about 1000-fold, about 10,000-fold or more as compared with the starting material.

As used herein, by “isolate” or “purify” (or grammatical equivalents) a virus vector, it is meant that the virus vector is at least partially separated from at least some of the other components in the starting material. In some embodiments an “isolated” or “purified” virus vector is enriched by at least about 10-fold, 100-fold, 1000-fold, 10,000-fold or more as compared with the starting material.

As used herein, the term “transduction-associated peptide” refers to a short amino acid sequence that may be incorporated into an AAV vector to alter the transduction of the AAV vector into any cell. The transduction-associated peptide may have any effect on the transduction of the AAV vector. For instance, in some embodiments, the transduction-associated peptide increases the transduction of the AAV vector into a target cell of interest. In some embodiments, the transduction-associated peptide decreases the transduction of the AAV vector into a cell that is not being targeted. The transduction-associated peptide may be inserted into an existing AAV capsid sequence (i.e., to produce a net addition of amino acids in the sequence), or it may replace an existing portion of an AAV capsid sequence (i.e., to produce no net change, or a reduction, in the number of amino acids in the sequence).

A “therapeutic polypeptide” or “therapeutic protein” is a polypeptide that can alleviate, reduce, prevent, delay and/or stabilize symptoms that result from an absence or defect in a protein in a cell or subject and/or is a polypeptide that otherwise confers a benefit to a subject, e.g., anti-cancer effects or improvement in transplant survivability.

By the terms “treat,” “treating” or “treatment of” (and grammatical variations thereof) it is meant that the severity of the subject's condition is reduced, at least partially improved or stabilized and/or that some alleviation, mitigation, decrease or stabilization in at least one clinical symptom is achieved and/or there is a delay in the progression of the disease or disorder. The term “subject” and the term “patient” are used interchangeably herein.

The terms “prevent,” “preventing” and “prevention” (and grammatical variations thereof) refer to prevention and/or delay of the onset of a disease, disorder and/or a clinical symptom(s) in a subject and/or a reduction in the severity of the onset of the disease, disorder and/or clinical symptom(s) relative to what would occur in the absence of the methods of the disclosure. The prevention can be complete, e.g., the total absence of the disease, disorder and/or clinical symptom(s). The prevention can also be partial, such that the occurrence of the disease, disorder and/or clinical symptom(s) in the subject and/or the severity of onset is less than what would occur in the absence of the present disclosure.

“Therapeutically effective amount” as used herein refers to an amount that, when administered to a subject for treating a disease, or at least one of the clinical symptoms of a disease, is sufficient to affect such treatment of the disease or symptom thereof. The “therapeutically effective amount” may vary depending, for example, on the disease and/or symptoms of the disease, severity of the disease and/or symptoms of the disease or disorder, the age, weight, and/or health of the patient to be treated, and the judgment of the prescribing physician. An appropriate amount in any given instance may be ascertained by those skilled in the art or capable of determination by routine experimentation.

As used herein, the terms “virus vector,” “vector” or “gene delivery vector” refer to a virus (e.g., AAV) particle that functions as a nucleic acid delivery vehicle, and which comprises the vector genome (e.g., viral DNA [vDNA]) packaged within a virion. Alternatively, in some contexts, the term “vector” may be used to refer to the vector genome/vDNA alone.

An “adeno-associated virus vector” or “AAV vector” typically comprises an AAV capsid, and a nucleic acid (e.g., a nucleic acid comprising a transgene) encapsidated by the AAV capsid. The “AAV capsid” is a near-spherical protein shell that comprises about 60 “AAV capsid proteins” (interchangeably referred to herein as, “AAV capsid protein subunits” or “capsid proteins”) associated and arranged with T=1 icosahedral symmetry. The AAV capsids of the AAV vectors described herein comprise a plurality of AAV capsid proteins. When an AAV vector is described as comprising an AAV capsid protein, it will be understood that the AAV vector comprises an AAV capsid, wherein the AAV capsid comprises one or more AAV capsid proteins. The term “viral-like particle” or “virus-like particle” refers to a protein capsid that does not comprise any vector genome or nucleic acid comprising a transfer cassette or transgene. The terms “AAV vector”, “AAV capsid” and “AAV capsid protein” may sometimes be used interchangeably herein. Based on the context, one of ordinary skill in the art will readily be able to deduce the meaning of the particular term used.

In some embodiments, an AAV vector may comprise a nucleic acid comprising a “transfer cassette,” i.e., a nucleic acid comprising one or more sequences which can be delivered by the AAV vector to a cell. In some embodiments, the nucleic acid is self-complementary (i.e., double stranded). In some embodiments, the nucleic acid is not self-complimentary (i.e., single stranded).

A “rAAV vector genome” or “rAAV genome” is an AAV genome (i.e., vDNA) that comprises one or more heterologous nucleic acid sequences. rAAV vectors generally require only the terminal repeat(s) (TR(s)) in cis to generate virus. All other viral sequences are dispensable and may be supplied in trans (Muzyczka, (1992) Curr. Topics Microbiol. Immunol. 158:97). Typically, the rAAV vector genome will only retain the one or more TR sequence so as to maximize the size of the transgene that can be efficiently packaged by the vector. The structural and non-structural protein coding sequences may be provided in trans (e.g., from a vector, such as a plasmid, or by stably integrating the sequences into a packaging cell). In embodiments, the rAAV vector genome comprises at least one TR sequence (e.g., AAV TR sequence), optionally two TRs (e.g., two AAV TRs), which typically will be at the 5′ and 3′ ends of the vector genome and flank the heterologous nucleic acid, but need not be contiguous thereto. The TRs can be the same or different from each other.

The term “terminal repeat” or “TR” includes any viral terminal repeat or synthetic sequence that forms a hairpin structure and functions as an inverted terminal repeat (i.e., mediates the desired functions such as replication, virus packaging, integration and/or provirus rescue, and the like). The TR can be an AAV TR or a non-AAV TR. For example, a non-AAV TR sequence such as those of other parvoviruses (e.g., canine parvovirus (CPV), mouse parvovirus (MVM), human parvovirus B-19) or any other suitable virus sequence (e.g., the SV40 hairpin that serves as the origin of SV40 replication) can be used as a TR, which can further be modified by truncation, substitution, deletion, insertion and/or addition. Further, the TR can be partially or completely synthetic, such as the “double-D sequence” as described in United States Patent No. 5,478,745 to Samulski et al.

An “AAV terminal repeat” or “AAV TR” may be from any AAV, including but not limited to serotypes 1, 2, 3, 4, 5, 6, 7, 8, 9, 10, 11, 12, 13 or any other AAV now known or later discovered (see, e.g., Table 2). An AAV terminal repeat need not have the native terminal repeat sequence (e.g., a native AAV TR sequence may be altered by insertion, deletion, truncation and/or missense mutations), as long as the terminal repeat mediates the desired functions, e.g., replication, virus packaging, integration, and/or provirus rescue, and the like.

The virus vectors of the disclosure can further be “targeted” virus vectors (e.g., having a directed tropism) and/or a “hybrid” parvovirus (i.e., in which the viral TRs and viral capsid are from different parvoviruses) as described in international patent publication WO00/28004 and Chao et al, (2000) Molecular Therapy 2:619.

The virus vectors of the disclosure can further be duplexed parvovirus particles as described in international patent publication WO 01/92551 (the disclosure of which is incorporated herein by reference in its entirety). Thus, in some embodiments, double stranded (duplex) genomes can be packaged into the virus capsids of the disclosure.

Further, the viral capsid or genomic elements can contain other modifications, including insertions, deletions and/or substitutions.

As used herein, the term “amino acid” encompasses any naturally occurring amino acid, modified forms thereof, and synthetic amino acids.

Naturally occurring, levorotatory (L-) amino acids are shown in Table 3.

TABLE 3
Amino acid residues and abbreviations.

| | Abbreviation | |
|---|---|---|
| Amino Acid Residue | Three-Letter Code | One-Letter Code |
| Alanine | Ala | A |
| Arginine | Arg | R |
| Asparagine | Asn | N |
| Aspartic acid (Aspartate) | Asp | D |
| Cysteine | Cys | C |
| Glutamine | Gln | Q |
| Glutamic acid (Glutamate) | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

Alternatively, the amino acid can be a modified amino acid residue (nonlimiting examples are shown in Table 4) and/or can be an amino acid that is modified by post-translation modification (e.g., acetylation, amidation, formylation, hydroxylation, methylation, phosphorylation or sulfatation).

TABLE 4
Modified Amino Acid Residues

| | |
|---|---|
| Modified Amino Acid Residue | Abbreviation |

Amino Acid Residue Derivatives

| | |
|---|---|
| 2-Aminoadipic acid | Aad |
| 3-Aminoadipic acid | bAad |
| beta-Alanine, beta-Aminoproprionic acid | bAla |
| 2-Aminobutyric acid | Abu |
| 4-Aminobutyric acid, Piperidinic acid | 4Abu |
| 6-Aminocaproic acid | Acp |
| 2-Aminoheptanoic acid | Ahe |
| 2-Aminoisobutyric acid | Aib |
| 3-Aminoisobutyric acid | bAib |
| 2-Aminopimelic acid | Apm |
| t-butylalanine | t-BuA |
| Citrulline | Cit |
| Cyclohexylalanine | Cha |
| 2,4-Diaminobutyric acid | Dbu |
| Desmosine | Des |
| 2,21-Diaminopimelic acid | Dpm |
| 2,3-Diaminoproprionic acid | Dpr |
| N-Ethylglycine | EtGly |
| N-Ethylasparagine | EtAsn |
| Homoarginine | hArg |
| Homocysteine | hCys |
| Homoserine | hSer |
| Hydroxylysine | Hyl |
| Allo-Hydroxylysine | aHyl |
| 3-Hydroxyproline | 3Hyp |
| 4-Hydroxyproline | 4Hyp |
| Isodesmosine | Ide |
| allo-Isoleucine | aIle |
| Methionine sulfoxide | MSO |
| N-Methylglycine, sarcosine | MeGly |
| N-Methyl isoleucine | MeIle |
| 6-N-Methyllysine | MeLys |
| N-Methylvaline | MeVal |
| 2-Naphthylalanine | 2-Nal |
| Norvaline | Nva |
| Norleucine | Nle |
| Ornithine | Orn |
| 4-Chlorophenylalanine | Phe(4-C1) |
| 2-Fluorophenylalanine | Phe(2-F) |
| 3-Fluorophenylalanine | Phe(3-F) |
| 4-Fluorophenylalanine | Phe(4-F) |
| Phenylglycine | Phg |
| Beta-2-thienylalanine | Thi |

Further, the non-naturally occurring amino acid can be an “unnatural” amino acid (as described by Wang et al., Annu Rev Biophys Biomol Struct. 35:225-49 (2006)). These unnatural amino acids can advantageously be used to chemically link molecules of interest to the AAV capsid protein.

An “active immune response” or “active immunity” is characterized by “participation of host tissues and cells after an encounter with the immunogen. It involves differentiation and proliferation of immunocompetent cells in lymphoreticular tissues, which lead to synthesis of antibody or the development of cell-mediated reactivity, or both.” Herbert B. Herscowitz, Immunophysiology: Cell Function and Cellular Interactions in Antibody Formation, in IMMUNOLOGY: BASIC PROCESSES 1 17 (Joseph A. Bellanti ed., 1985). Alternatively stated, an active immune response is mounted by the host after exposure to an immunogen by infection or by vaccination. Active immunity can be contrasted with passive immunity, which is acquired through the transfer of preformed substances (antibody, transfer factor, thymic graft, interleukin-2) from an actively immunized host to a non-immune host.

A “protective” immune response or “protective” immunity as used herein indicates that the immune response confers some benefit to the subject in that it prevents or reduces the incidence of disease. Alternatively, a protective immune response or protective immunity may be useful in the treatment and/or prevention of disease, in particular cancer or tumors (e.g., by preventing cancer or tumor formation, by causing regression of a cancer or tumor and/or by preventing metastasis and/or by preventing growth of metastatic nodules). The protective effects may be complete or partial, as long as the benefits of the treatment outweigh any disadvantages thereof.

As used herein, the term “cancer” encompasses tumor-forming cancers. Likewise, the term “cancerous tissue” encompasses tumors. A “cancer cell antigen” encompasses tumor antigens.

The term “cancer” has its understood meaning in the art, for example, an uncontrolled growth of tissue that has the potential to spread to distant sites of the body (i.e., metastasize). Exemplary cancers include, but are not limited to melanoma, adenocarcinoma, thymoma, lymphoma (e.g., non-Hodgkin's lymphoma, Hodgkin's lymphoma), sarcoma, lung cancer, liver cancer, colon cancer, leukemia, uterine cancer, breast cancer, prostate cancer, ovarian cancer, cervical cancer, bladder cancer, kidney cancer, pancreatic cancer, brain cancer and any other cancer or malignant condition now known or later identified. In representative embodiments, the disclosure provides a method of treating and/or preventing tumor-forming cancers.

The term “tumor” is also understood in the art, for example, as an abnormal mass of undifferentiated cells within a multicellular organism. Tumors can be malignant or benign. In representative embodiments, the methods disclosed herein are used to prevent and treat malignant tumors.

By the terms “treating cancer,” “treatment of cancer” and equivalent terms it is intended that the severity of the cancer is reduced or at least partially eliminated and/or the progression of the disease is slowed and/or controlled and/or the disease is stabilized. In some embodiments, these terms indicate that metastasis of the cancer is prevented or reduced or at least partially eliminated and/or that growth of metastatic nodules is prevented or reduced or at least partially eliminated.

By the terms “prevention of cancer” or “preventing cancer” and equivalent terms it is intended that the methods at least partially eliminate or reduce and/or delay the incidence and/or severity of the onset of cancer. Alternatively stated, the onset of cancer in the subject may be reduced in likelihood or probability and/or delayed.

### Modified AAV Capsid Proteins and Capsids Comprising the Same

The present disclosure provides AAV capsid protein (VP1, VP2 and/or VP3) variants, and virus capsids and virus vectors comprising the same. Each capsid variant comprises one or more transduction-associated peptides. The transduction-associated peptides are not present in a naturally occurring AAV capsid protein and may, in some embodiments, confer enhanced transduction to an AAV vector comprising the capsid protein into a target cell of interest (e.g., a T-cell). The AAV capsid protein variants disclosed herein may be variants relative to the capsid proteins of any AAV serotype now known or later discovered. In some embodiments, the AAV capsid protein variant is a variant of a capsid protein from an AAV serotype selected from AAV1, AAV2, AAV3, AAV4, AAV5, AAV6, AAV7, AAV8, AAV9, AAV10, AAV11, AAV12, AAVrh.8, AAVrh.10, AAVrh32.33, AAVrh74, bovine AAV and avian AAV.

a. Modifications of AAV Capsid Proteins

In some embodiments, the transduction-associated peptides described herein can confer one or more desirable properties to virus vectors comprising the modified AAV capsid protein including without limitation, enhanced cellular transduction in various cell types (e.g., T-cells), in vitro, in vivo or ex vivo. In some embodiments, the capsid proteins of the disclosure may be incorporated into an AAV vector. In some embodiments, the AAV vector comprising the capsid protein has enhanced cellular transduction (e.g. enhanced T-cell transduction), compared to a wild type AAV or an AAV virus particle or AAV virus vector comprising an AAV capsid protein that does not comprise the transduction-associated peptide. In some embodiments, an AAV virus particle or vector of this disclosure can also evade neutralizing antibodies.

The transduction-associated peptides of the disclosure may replace an amino acid sequence of a wild type AAV capsid protein, resulting in no net increase or decrease of the number of amino acids in the AAV capsid protein sequence. In some embodiments, replacement of an amino acid sequence of a wild type AAV capsid protein with a transduction-associated peptide of the disclosure may result in a net loss of amino acids (e.g., a deletion) compared to the wild type AAV capsid protein sequence. For example, the transduction-associated peptide may replace one or more amino acids in an AAV capsid protein from any one of the following serotypes: AAV1, AAV2, AAV3, AAV4, AAV5, AAV6, AAV7, AAV8, AAV9, AAV10, AAV11, AAV12, AAVrh.8, AAVrh.10, AAVrh32.33, AAVrh74, bovine AAV and avian AAV. In some embodiments, the transduction-associated peptides of the disclosure may be inserted into an amino acid sequence of a wild type AAV capsid protein, resulting in an increase in the number of amino acids in the AAV capsid protein sequence.

In some embodiments, modification of the AAV capsid protein results in replacement of one or more amino acid residues of a native AAV capsid protein with an amino acid that does not occur in the native capsid sequence. In some embodiments, modification of the AAV capsid protein results in replacement of one or more of the following amino acid residues: 454, 455, 456, 457, 458, 459, and 460, with an amino acid that does not occur in the native capsid protein sequence, wherein the amino acid numbering is relative to the VP1 sequence of the wildtype AAV6 capsid protein, or the corresponding residues in the capsid protein of any other AAV serotype. In some embodiments, modification of the AAV capsid protein results in a deletion of one or more of the following amino acid residues: 454, 455, 456, 457, 458, 459, and 460, wherein the amino acid numbering is relative to the VP1 sequence of the wildtype AAV6 capsid protein, or the corresponding residues in the capsid protein of any other AAV serotype. In some embodiments, modification of the AAV capsid protein results in replacement of one or more of the amino acids 454, 455, 456, 457, 458, 459, and/or 460 relative to the amino acid sequence of the native AAV6 capsid protein sequence (SEQ ID NO: 1).

In some embodiments, an AAV capsid protein comprises a transduction-associated peptide of the sequence X1-X2-X3-X4-X5-X6-X7 (SEQ ID NO: 24). In some embodiments, an AAV capsid protein comprises a transduction-associated peptide of the sequence X1-X2-X3-X4-X5-X6-X7 (SEQ ID NO: 24), wherein the capsid protein is of any one of the following serotypes: AAV1, AAV2, AAV3, AAV4, AAV5, AAV6, AAV7, AAV8, AAV9, AAV10, AAV11, AAV12, AAVrh.8, AAVrh.10, AAVrh32.33, AAVrh74, bovine AAV or avian AAV. In some embodiments, an AAV capsid protein comprising an amino acid sequence selected from any one of SEQ ID NOs: 1 or 25-34 comprises a transduction-associated peptide of the sequence X1-X2-X3-X4-X5-X6-X7 (SEQ ID NO: 24). In some embodiments, the AAV capsid protein comprises the sequence of the native AAV6 capsid protein sequence (e.g., SEQ ID NO: 1), and further, comprises a transduction-associated peptide of the SEQ ID NO: 24. In some embodiments, an AAV capsid protein comprises an amino acid sequence that has at least about 80% identity, for example, at least about 85%, at least about 90%, at least about 95%, at least about 96%, at least about 97%, at least about 98%, at least about 99%, at least about 99.5%, or about 100% identity, to the amino acid sequence of a wild type AAV capsid protein sequence, such as, for example, SEQ ID NO: 1, or 25-34. In some embodiments, the AAV capsid proteins disclosed herein comprise an amino acid sequence having about 99% identity to SEQ ID NO: 1.

The transduction-associated peptide of SEQ ID NO: 24 may be used to replace a one or more amino acid residues anywhere in the amino acid sequence of the disclosed AAV capsid proteins. In some embodiments, the transduction-associated peptide of SEQ ID NO: 24 may be used to replace a sequence in a capsid protein, wherein the capsid protein has an amino acid sequence selected from any one of SEQ ID NOs: 1 and 25-34. In some embodiments, the transduction-associated peptide of the sequence SEQ ID NO: 24 may be inserted into the amino acid sequence of the AAV capsid proteins disclosed herein. In some embodiments, replacement of a native sequence of one or more AAV capsid proteins described herein with the transduction-associated peptide of the sequence SEQ ID NO: 24 may result in the deletion of one or more amino acids from the sequence of the AAV capsid protein. In some embodiments, a capsid protein may comprise the sequence of SEQ ID NO: 1, except that amino acids 454-460 of SEQ ID NO: 1 are replaced by a transduction-associated peptide comprising the sequence SEQ ID NO: 24. In some embodiments, SEQ ID NO: 24 is used to replace a sequence of a wild type AAV capsid protein, such that the resulting sequence comprises at least one, two, three, etc., individual amino acids that do not occur in the wild type sequence.

In some embodiments, SEQ ID NO: 24 comprises a sequence wherein X1 is not G, X2 is not S, X3 is not A, X4 is not Q, X5 is not N, X6 is not K, and/or X7 is not D. In some embodiments, X1 is H, M, A, Q, V, or S. In some embodiments, X2 is A or T. In some embodiments, X3 is P or T. In some embodiments, X4 is R or D. In some embodiments, X5 is V, Q, C, S, or D. In some embodiments, X6 is E, A, or P. In some embodiments, X7 is E, G, N, T, or A. In some embodiments, X1 is H, X2 is A, X3 is P, X4 is R, X5 is V, X6 is E, and X7 is E. In some embodiments, X1 is M, X2 is A, X3 is P, X4 is R, X5 is Q, X6 is E, and X7 is G. In some embodiments, X1 is H, X2 is T, X3 is T, X4 is D, X5 is C, X6 is A, and X7 is N. In some embodiments, X1 is A, X2 is A, X3 is P, X4 is R, X5 is S, X6 is E, and X7 is T. In some embodiments, X1 is Q, X2 is A, X3 is P, X4 is R, X5 is Q, X6 is E, and X7 is G. In some embodiments, X1 is V, X2 is A, X3 is P, X4 is R, X5 is D, X6 is P, and X7 is A. In some embodiments, X1 is S, X2 is A, X3 is P, X4 is R, X5 is S, X46 is E, and X7 is N.

In some embodiments, the transduction-associated peptide has an amino acid sequence of X1-X2-X3-X4-X5-X6-X7, wherein X1=H, M, Q, V or S; X2=A or T; X3=P or T; X4=R or D; X5=V, Q, C, S, or D, X6=E, A or P; and X7=E, G, N, T or A (SEQ ID NO: 16). In some embodiments, the transduction-associated peptide has an amino acid sequence of any one of SEQ ID NOs: 17-23.

In some embodiments, an AAV capsid protein comprises a transduction-associated peptide having an amino acid sequence of any one of SEQ ID NOs: 17-23. In some embodiments, a transduction-associated peptide having an amino acid sequence of any one of SEQ ID NOs: 17-23 replaces one or more amino acids of an AAV capsid protein. The disclosure provides variants of AAV capsid proteins of any one of the following serotypes: AAV1, AAV2, AAV3, AAV4, AAV5, AAV6, AAV7, AAV8, AAV9, AAV10, AAV11, AAV12, AAVrh.8, AAVrh.10, AAVrh32.33, AAVrh74, bovine AAV and avian AAV, wherein the AAV capsid protein variant comprises an amino acid sequence comprising a transduction-associated peptide having an amino acid sequence of any one of SEQ ID NOs: 17-23. In some embodiments, an AAV capsid protein comprises an amino acid sequence selected from any one of SEQ ID NOs: 1 and 25-34 but wherein one or more amino acids are replaced with a transduction-associated peptide having an amino acid sequence of any one of SEQ ID NOs: 17-23.

In some embodiments, a transduction-associated peptide having an amino acid sequence of any one of SEQ ID NOs: 17-23 replaces one or more amino acids of an AAV capsid protein of any one of the following serotypes: AAV1, AAV2, AAV3, AAV4, AAV5, AAV6, AAV7, AAV8, AAV9, AAV10, AAV11, AAV12, AAVrh.8, AAVrh.10, AAVrh32.33, AAVrh74, bovine AAV and avian AAV. In some embodiments, a transduction-associated peptide having an amino acid sequence of any one of SEQ ID NOs: 17-23 replaces one or more amino acids of an AAV capsid protein comprising an amino acid sequence selected from any one of SEQ ID NOs: 1 and 25-34.

In some embodiments, amino acids 454-460 of the native AAV6 capsid protein (e.g. SEQ ID NO: 1) are replaced by a transduction-associated peptide comprising the sequence any one of SEQ ID NOs: 17-23. In some embodiments, amino acids 454-460 of the native AAV6 capsid protein (e.g. SEQ ID NO: 1) are replaced by a transduction-associated peptide of the sequence SEQ ID NO: 17. In some embodiments, amino acids 454-460 of the native AAV6 capsid protein (e.g. SEQ ID NO: 1) are replaced by a transduction-associated peptide of the sequence SEQ ID NO: 18. In some embodiments, amino acids 454-460 of the native AAV6 capsid protein (e.g. SEQ ID NO: 1) are replaced by a transduction-associated peptide of the sequence SEQ ID NO: 19. In some embodiments, amino acids 454-460 of the native AAV6 capsid protein (e.g. SEQ ID NO: 1) are replaced by a transduction-associated peptide of the sequence SEQ ID NO: 20. In some embodiments, amino acids 454-460 of the native AAV6 capsid protein (e.g. SEQ ID NO: 1) are replaced by a transduction-associated peptide of the sequence SEQ ID NO: 21. In some embodiments, amino acids 454-460 of the native AAV6 capsid protein (e.g. SEQ ID NO: 1) are replaced by a transduction-associated peptide of the sequence SEQ ID NO: 22. In some embodiments, amino acids 454-460 of the native AAV6 capsid protein (e.g. SEQ ID NO: 1) are replaced by a transduction-associated peptide of the sequence SEQ ID NO: 23.

In some embodiments, an AAV capsid protein comprises an amino acid sequence selected from the group consisting of SEQ ID NOs: 2, 4, 6, 8, 10, 12, and 14, or a sequence at least about 80% identical thereto. For example, in some embodiments, an AAV capsid protein comprises an amino acid sequence that is at least about 85%, at least about 90%, at least about 95%, at least about 96%, at least about 97%, at least about 98%, at least about 99% identity, at least about 99.5%, or about 100% identical to any one of SEQ ID NOs: 2, 4, 6, 8, 10, 12, or 14.

b. Other Modifications of AAV Capsid Proteins

The disclosure contemplates that the AAV capsid protein that is to be modified can be a naturally occurring AAV capsid protein (e.g., an AAV2, AAV3a or 3b, AAV4, AAV5, AAV6, AAV7, AAV8, AAV9, AAV10 or AAV11 capsid protein or any of the AAV shown in Table 2) but is not so limited. Those skilled in the art will understand that a variety of manipulations to the AAV capsid proteins are known in the art and the disclosure is not limited to modifications of naturally occurring AAV capsid proteins. For example, the capsid protein to be modified may already have alterations as compared with naturally occurring AAV (e.g., is derived from a naturally occurring AAV capsid protein, e.g., AAV2, AAV3a, AAV3b, AAV4, AAV5, AAV6, AAV7, AAV8, AAV9, AAV10, AAV11, AAV12 or any other AAV now known or later discovered). In some embodiments, the capsid protein may be an engineered AAV, such as AAV2i8, AAV2g9, AAV-LK03, AAV7m8, AAV Anc80, AAV PHP.B. Such AAV capsid proteins are also within the scope of the present disclosure.

In some embodiments, the AAV capsid protein is chimeric. For example, the chimeric AAV capsid protein may comprise sequences derived from two or more AAV serotypes, or three or more AAV serotypes. The chimeric AAV capsid protein may comprise sequences derived from two or more of the following AAV serotypes: AAV1, AAV2, AAV3, AAV4, AAV5, AAV6, AAV7, AAV8, AAV9, AAV10, AAV11, AAV12, AAVrh.8, AAVrh.10, AAVrh32.33, AAVrh74, bovine AAV and avian AAV.

Thus, in some embodiments, the AAV capsid protein to be modified can be derived from a naturally occurring AAV but further comprises one or more foreign sequences (e.g., that are exogenous to the native virus) that are inserted and/or substituted into the capsid protein and/or has been altered by deletion of one or more amino acids. Accordingly, when referring herein to a specific AAV capsid protein (e.g., an AAV1, AAV2, AAV3, AAV4, AAV5, AAV6, AAV7, AAV8, AAV9, AAV10 or AAV11 capsid protein or a capsid protein from any of the AAV shown in Table 2, etc.), it is intended to encompass the native capsid protein as well as capsid proteins that have alterations other than the modifications of the disclosure. Such alterations include substitutions, insertions and/or deletions. In some embodiments, the capsid protein comprises 1, 2, 3, 4, 5, 6, 7, 8, 9, 10, 11, 12, 13, 14, 15, 16, 17, 18, 19 or 20, less than 20, less than 30, less than 40, less than 50, less than 60, or less than 70 amino acids inserted therein (other than the amino acid sequence substitutions of the present disclosure) as compared with the native AAV capsid protein sequence. In embodiments, the capsid protein comprises 1, 2, 3, 4, 5, 6, 7, 8, 9, 10, 11, 12, 13, 14, 15, 16, 17, 18, 19 or 20, less than 20, less than 30, less than 40, less than 50, less than 60, or less than 70 amino acid substitutions (other than the transduction-associated peptides according to the present disclosure) as compared with the native AAV capsid protein sequence. In some embodiments, the capsid protein comprises a deletion of 1, 2, 3, 4, 5, 6, 7, 8, 9, 10, 11, 12, 13, 14, 15, 16, 17, 18, 19 or 20, less than 20, less than 30, less than 40, less than 50, less than 60, or less than 70 amino acids (other than the transduction-associated peptides of the disclosure) as compared with the native AAV capsid protein sequence.

The modifications to the AAV capsid protein according to the present disclosure are “selective” modifications. This approach is in contrast to previous work with whole subunit or large domain swaps between AAV serotypes (see, e.g., international patent publication WO 00/28004 and Hauck et al., (2003) J. Virology 77:2768-2774). In some embodiments, a “selective” modification results in the insertion and/or substitution and/or deletion of less than or equal to about 20, 18, 15, 12, 10, 9, 8, 7, 6, 5, 4 or 3 contiguous amino acids. The modified capsid proteins and capsids of the disclosure can further comprise any other modification, now known or later identified. In the embodiments described herein wherein an amino acid residue is substituted by any amino acid residue other than the amino acid residue present in the wild type or native amino acid sequence, said any other amino acid residue can be any natural or non-natural amino acid residue known in the art (see, e.g., Tables 3 and 4). In some embodiments, the substitution can be a conservative substitution and in some embodiments, the substitution can be a non-conservative substitution.

As described herein, the amino acid sequences and the nucleic acid sequences of the capsid proteins from a number of AAVs are known in the art. Thus, the amino acids “corresponding” to amino acid positions of the native AAV capsid protein can be readily determined for any other AAV (e.g., by using sequence alignments). Methods of determining sequence similarity or identity between two or more amino acid sequences are known in the art. Sequence similarity or identity may be determined using standard techniques known in the art, including, but not limited to, the local sequence identity algorithm of Smith & Waterman, Adv. Appl. Math. 2, 482 (1981), by the sequence identity alignment algorithm of Needleman & Wunsch, J Mol. Biol. 48,443 (1970), by the search for similarity method of Pearson & Lipman, Proc. Natl. Acad. Sci. USA 85,2444 (1988), by computerized implementations of these algorithms (GAP, BESTFIT, FASTA, and TFASTA in the Wisconsin Genetics Software Package, Genetics Computer Group, 575 Science Drive, Madison, WI), the Best Fit sequence program described by Devereux et al., Nucl. Acid Res. 12, 387-395 (1984), or by inspection.

Another suitable algorithm is the BLAST algorithm, described in Altschul et al., J Mol. Biol. 215, 403-410, (1990) and Karlin et al., Proc. Natl. Acad. Sci. USA 90, 5873-5787 (1993). A particularly useful BLAST program is the WU-BLAST-2 program which was obtained from Altschul et al., Methods in Enzymology, 266, 460-480 (1996); blast.wustl/edu/blast/README.html. WU-BLAST-2 uses several search parameters, which are optionally set to the default values. The parameters are dynamic values and are established by the program itself depending upon the composition of the particular sequence and composition of the particular database against which the sequence of interest is being searched; however, the values may be adjusted to increase sensitivity.

Further, an additional useful algorithm is gapped BLAST as reported by Altschul et al, (1997) Nucleic Acids Res. 25, 3389-3402.

Unless indicated otherwise, calculation of percent identity is performed in the instant disclosure using the BLAST algorithm available at the world wide web address: blast.ncbi.nlm.nih.gov/Blast.cgi.

c. Modified Viral Capsids

The disclosure also provides virus capsids comprising at least one of the variant capsid proteins disclosed herein. In some embodiments, the virus capsid is a parvovirus capsid, which may further be an autonomous parvovirus capsid or a dependovirus capsid. Optionally, the virus capsid is an AAV capsid. In some embodiments, the AAV capsid is an AAV1, AAV2, AAV3a, AAV3b, AAV4, AAV5, AAV6, AAV7, AAV8, AAV9, AAV10, AAV11, AAV12, AAVrh8, AAVrh10, AAVrh32.33, bovine AAV capsid, avian AAV capsid or any other AAV now known or later identified. A nonlimiting list of AAV serotypes is shown in Table 2. An AAV capsid of this disclosure can be any AAV serotype listed in Table 2 or derived from any of the foregoing by one or more insertions, substitutions and/or deletions. The modified virus capsids can be used as “capsid vehicles,” as has been described, for example, in U.S. Pat. No. 5,863,541. Virus capsids according to the disclosure can be produced using any method known in the art, e.g., by expression from a baculovirus (Brown et al., (1994) Virology 198:477-488). In some embodiments, an AAV capsid comprises about 60 variant capsid proteins described herein.

In some embodiments, the virus capsid can be a targeted virus capsid comprising a targeting sequence (e.g., substituted or inserted in the viral capsid) that directs the virus capsid to interact with cell-surface molecules present on desired target tissue(s) (see, e.g., International patent publication WO 00/28004 and Hauck et al., (2003) J. Virology 77:2768-2774); Shi et al., Human Gene Therapy 17:353-361 (2006) [describing insertion of the integrin receptor binding motif RGD at positions 520 and/or 584 of the AAV capsid subunit]; and U.S. Pat. No. 7,314,912 [describing insertion of the PI peptide containing an RGD motif following amino acid positions 447, 534, 573 and 587 of the AAV2 capsid subunit]). Other positions within the AAV capsid subunit that tolerate insertions are known in the art (e.g., positions 449 and 588 described by Grifman et al., Molecular Therapy 3:964-975 (2001)).

For example, a virus capsid of this disclosure may have relatively inefficient tropism toward certain target tissues of interest (e.g., liver, skeletal muscle, heart, diaphragm muscle, kidney, brain, stomach, intestines, skin, endothelial cells, and/or lungs). A targeting sequence can advantageously be incorporated into these low-transduction vectors to thereby confer to the virus capsid a desired tropism and, optionally, selective tropism for particular tissues or cells, such as T-cells. AAV capsid proteins, capsids and vectors comprising targeting sequences are described, for example in international patent publication WO 00/28004. As another example, one or more non-naturally occurring amino acids as described by Wang et al., Annu Rev Biophys Biomol Struct. 35:225-49 (2006)) can be incorporated into an AAV capsid subunit of this disclosure at an orthogonal site as a means of redirecting a low-transduction vector to desired target tissue(s). These unnatural amino acids can advantageously be used to chemically link molecules of interest to the AAV capsid protein including without limitation: glycans (mannose-dendritic cell targeting); RGD, bombesin or a neuropeptide for targeted delivery to specific cancer cell types; RNA aptamers or peptides selected from phage display targeted to specific cell surface receptors such as growth factor receptors, integrins, and the like. Methods of chemically modifying amino acids are known in the art (see, e.g., Greg T. Hermanson, Bioconjugate Techniques, 1^{st} edition, Academic Press, 1996). In some embodiments, the targeting sequence may be a virus capsid sequence (e.g., an autonomous parvovirus capsid sequence, AAV capsid sequence, or any other viral capsid sequence) that directs infection to a particular cell type(s).

As another nonlimiting example, a heparin or heparan sulfate (HS) binding domain (e.g., the respiratory syncytial virus heparin binding domain) may be inserted or substituted into a capsid subunit that does not typically bind HS receptors (e.g., AAV4, AAV5) to confer heparin and/or heparan sulfate binding to the resulting variant. It is known in the art that HS/heparin binding is mediated by a “basic patch” that is rich in arginines and/or lysines. In exemplary embodiments, a sequence following the motif BXXB (SEQ ID NO: 105), where “B” is a basic residue and X is neutral and/or hydrophobic can be employed. As a nonlimiting example, BXXB can be RGNR (SEQ ID NO: 106). As another nonlimiting example, BXXB is substituted for amino acid positions 262 through 265 in the native AAV2 capsid protein or at the corresponding position(s) in the capsid protein of another AAV serotype.

Parvovirus B19 infects primary erythroid progenitor cells using globoside as its receptor (Brown et al, (1993) Science 262: 114). The structure of B19 has been determined to 8 Å resolution (Agbandje-McKenna et al, (1994) Virology 203:106). The region of the B19 capsid that binds to globoside has been mapped between amino acids 399-406 (Chapman et al, 15 (1993) Virology 194:419), a looped out region between β-barrel structures E and F (Chipman et al, (1996) Proc. Nat. Acad. Sci. USA 93:7502). Accordingly, the globoside receptor binding domain of the B19 capsid may be substituted into an AAV capsid protein of this disclosure to target a virus capsid or virus vector comprising the same to erythroid cells.

In some embodiments, the exogenous targeting sequence may be any amino acid sequence encoding a peptide that alters the tropism of a virus capsid or virus vector comprising the modified AAV capsid protein. In some embodiments, the targeting peptide or protein may be naturally occurring or, alternately, completely or partially synthetic. Exemplary targeting sequences include ligands and other peptides that bind to cell surface receptors and glycoproteins, such as ROD peptide sequences, bradykinin, hormones, peptide growth factors (e.g., epidermal growth factor, nerve growth factor, fibroblast growth factor, platelet-derived growth factor, insulin-like growth factors I and II, etc.), cytokines, melanocyte stimulating hormone (e.g., a, β or γ), neuropeptides and endorphins, and the like, and fragments thereof that retain the ability to target cells to their cognate receptors. Other illustrative peptides and proteins include substance P, keratinocyte growth factor, neuropeptide Y, gastrin releasing peptide, interleukin 2, hen egg white lysozyme, erythropoietin, gonadoliberin, corticostatin, β-endorphin, leu-enkephalin, rimorphin, alpha-neo-enkephalin, angiotensin, pneumadin, vasoactive intestinal peptide, neurotensin, motilin, and fragments thereof as described above. As yet a further alternative, the binding domain from a toxin (e.g., tetanus toxin or snake toxins, such as alpha-bungarotoxin, and the like) can be substituted into the capsid protein as a targeting sequence. In a yet further representative embodiment, the AAV capsid protein can be modified by substitution of a “nonclassical” import/export signal peptide (e.g., fibroblast growth factor-1 and -2, interleukin 1, HIV-1 Tat protein, herpes virus VP22 protein, and the like) as described by Cleves (Current Biology 7:R318 (1997)) into the AAV capsid protein. Also encompassed are peptide motifs that direct uptake by specific cells, e.g., a FVFLP (SEQ ID NO: 104) peptide motif triggers uptake by liver cells.

Phage display techniques, as well as other techniques known in the art, may be used to identify peptides that recognize any cell type of interest. The targeting sequence may encode any peptide that targets to a cell surface binding site, including receptors (e.g., protein, carbohydrate, glycoprotein or proteoglycan). Examples of cell surface binding sites include, but are not limited to, heparan sulfate, chondroitin sulfate, and other glycosaminoglycans, sialic acid moieties found on mucins, glycoproteins, and gangliosides, MHC 1 glycoproteins, carbohydrate components found on membrane glycoproteins, including, mannose, N-acetyl-galactosamine, N-acetyl-glucosamine, fucose, galactose, and the like. Table 7 shows other non-limiting examples of suitable targeting sequences.

TABLE 7
Illustrative targeting sequences

| Sequence | SEQ ID NO | Reference |
|---|---|---|
| NSVRDL(G/S) | 35 | Muller et al., Nature Biotechnology 21: 1040-1046 |
| | | (2003) |
| PRSVTVP | 36 | Muller et al., Nature Biotechnology 21: 1040-1046 |
| | | (2003) |
| NSVSSX(S/A) | 37 | Muller et al., Nature Biotechnology 21: 1040-1046 |
| | | (2003) |
| NGR, NGRAHA | 38 | Grifman et al., Molecular Therapy 3:964-975 (2001) |
| QPEHSST | 39 | Work et al., Molecular Therapy 13:683-693 (2006) |
| VNTANST | 40 | Work et al., Molecular Therapy 13:683-693 (2006) |
| HGPMQS | 41 | Work et al., Molecular Therapy 13:683-693 (2006) |
| PHKPPLA | 42 | Work et al., Molecular Therapy 13:683-693 (2006) |
| IKNNEMW | 43 | Work et al., Molecular Therapy 13:683-693 (2006) |
| RNLDTPM | 44 | Work et al., Molecular Therapy 13:683-693 (2006) |
| VDSHRQS | 45 | Work et al., Molecular Therapy 13:683-693 (2006) |
| YDSKTKT | 46 | Work et al., Molecular Therapy 13:683-693 (2006) |
| SQLPHQK | 47 | Work et al., Molecular Therapy 13:683-693 (2006) |
| STMQQNT | 48 | Work et al., Molecular Therapy 13:683-693 (2006) |
| TERYMTQ | 49 | Work et al., Molecular Therapy 13:683-693 (2006) |
| QPEHSST | 50 | Work et al., Molecular Therapy 13:683-693 (2006) |
| DASLSTS | 51 | Work et al., Molecular Therapy 13:683-693 (2006) |
| DLPNKT | 52 | Work et al., Molecular Therapy 13:683-693 (2006) |
| DLTAARL | 53 | Work et al., Molecular Therapy 13:683-693 (2006) |
| EPHQFNY | 54 | Work et al., Molecular Therapy 13:683-693 (2006) |
| EPQSNHT | 55 | Work et al., Molecular Therapy 13:683-693 (2006) |
| MSSWPSQ | 56 | Work et al., Molecular Therapy 13:683-693 (2006) |
| NPKHNAT | 57 | Work et al., Molecular Therapy 13:683-693 (2006) |
| PDGMRTT | 58 | Work et al., Molecular Therapy 13:683-693 (2006) |
| PNNNKTT | 59 | Work et al., Molecular Therapy 13:683-693 (2006) |
| QSTTHDS | 60 | Work et al., Molecular Therapy 13:683-693 (2006) |
| TGSKQKQ | 61 | Work et al., Molecular Therapy 13:683-693 (2006) |
| SLKHQAL | 62 | Work et al., Molecular Therapy 13:683-693 (2006) |
| SPIDGEQ | 63 | Work et al., Molecular Therapy 13:683-693 (2006) |
| WIFPWIQL | 64 | Hajitou et al., TCM 16:80-88 (2006) |
| CDCRGDCFC | 65 | Hajitou et al., TCM 16:80-88 (2006) |
| CNGRC | 66 | Hajitou et al., TCM 16:80-88 (2006) |
| CPRECES | 67 | Hajitou et al., TCM 16:80-88 (2006) |
| CTTHWGFTLC | 68 | Hajitou et al., TCM 16:80-88 (2006) |
| CGRRAGGSC | 69 | Hajitou et al., TCM 16:80-88 (2006) |
| CKGGRAKDC | 70 | Hajitou et al., TCM 16:80-88 (2006) |
| CVPELGHEC | 71 | Hajitou et al., TCM 16:80-88 (2006) |
| CRRETAWAK | 72 | Koivunen et al., J. Nucl. Med. 40:883-888 (1999) |
| VSWFSHRYSPFAVS | 73 | Koivunen et al., J. Nucl. Med. 40:883-888 (1999) |
| GYRDGYAGPILYN | 74 | Koivunen et al., J. Nucl. Med. 40:883-888 (1999) |
| XXXY*XXX | 75 | Koivunen et al., J. Nucl. Med. 40:883-888 (1999) |
| Y*E/MNW | 76 | Koivunen et al., J. Nucl. Med. 40:883-888 (1999) |
| RPLPPLP | 77 | Koivunen et al., J. Nucl. Med. 40:883-888 (1999) |
| APPLPPR | 78 | Koivunen et al., J. Nucl. Med. 40:883-888 (1999) |
| DVFYPYPYASGS | 79 | Koivunen et al., J. Nucl. Med. 40:883-888 (1999) |
| MYWYPY | 80 | Koivunen et al., J. Nucl. Med. 40:883-888 (1999) |
| DITWDQLWDLMK | 81 | Koivunen et al., J. Nucl. Med. 40:883-888 (1999) |
| CWDD(G/L)WLC | 82 | Koivunen et al., J. Nucl. Med. 40:883-888 (1999) |
| EWCEYLGGYLRCYA | 83 | Koivunen et al., J. Nucl. Med. 40:883-888 (1999) |
| YXCXXGPXTWXCXP | 84 | Koivunen et al., J. Nucl. Med. 40:883-888 (1999) |
| IEGPTLRQWLAARA | 85 | Koivunen et al., J. Nucl. Med. 40:883-888 (1999) |
| LWXX(Y/W/F/H) | 86 | Koivunen et al., J. Nucl. Med. 40:883-888 (1999) |
| XFXXYLW | 87 | Koivunen et al., J. Nucl. Med. 40:883-888 (1999) |
| RWGLCD | 88 | Koivunen et al., J. Nucl. Med. 40:883-888 (1999) |
| MSRPACPPNDKYE | 89 | Koivunen et al., J. Nucl. Med. 40:883-888 (1999) |
| CLRSGRGC | 90 | Koivunen et al., J. Nucl. Med. 40:883-888 (1999) |
| CHWMFSPWC | 91 | Koivunen et al., J. Nucl. Med. 40:883-888 (1999) |
| WXXF | 92 | Koivunen et al., J. Nucl. Med. 40:883-888 (1999) |
| CSSRLDAC | 93 | Koivunen et al., J. Nucl. Med. 40:883-888 (1999) |
| CLPVASC | 94 | Koivunen et al., J. Nucl. Med. 40:883-888 (1999) |
| CGFECVRQCPERC | 95 | Koivunen et al., J. Nucl. Med. 40:883-888 (1999) |
| CVALCREACGEGC | 96 | Koivunen et al., J. Nucl. Med. 40:883-888 (1999) |
| SWCEPGWCR | 97 | Koivunen et al., J. Nucl. Med. 40:883-888 (1999) |
| YSGWGW | 98 | Koivunen et al., J. Nucl. Med. 40:883-888 (1999) |
| GLSGGRS | 99 | Koivunen et al., J. Nucl. Med. 40:883-888 (1999) |
| LMLPRAD | 100 | Koivunen et al., J. Nucl. Med. 40:883-888 (1999) |
| CSCFRDVCC | 101 | Koivunen et al., J. Nucl. Med. 40:883-888 (1999) |
| CRDVVSVIC | 102 | Koivunen et al., J. Nucl. Med. 40:883-888 (1999) |
| CNGRC | 103 | Koivunen et al., J. Nucl. Med. 40:883-888 (1999) |
| MARSGL | 104 | Newton & Deutscher, Phage Peptide Display in |
| | | Handbook of Experimental Pharmacology, pages |
| | | 145-163, Springer-Verlag, Berlin (2008) |
| MARAKE | 105 | Newton & Deutscher, Phage Peptide Display in |
| | | Handbook of Experimental Pharmacology, pages |
| | | 145-163, Springer-Verlag, Berlin (2008) |
| MSRTMS | 106 | Newton & Deutscher, Phage Peptide Display in |
| | | Handbook of Experimental Pharmacology, pages |
| | | 145-163, Springer-Verlag, Berlin (2008) |
| KCCYSL | 107 | Newton & Deutscher, Phage Peptide Display in |
| | | Handbook of Experimental Pharmacology, pages |
| | | 145-163, Springer-Verlag, Berlin (2008) |
| MYWGDSHWLQYW | 108 | Newton & Deutscher, Phage Peptide Display in |
| YE | | Handbook of Experimental Pharmacology, pages |
| | | 145-163, Springer-Verlag, Berlin (2008) |
| MQLPLAT | 109 | Newton & Deutscher, Phage Peptide Display in |
| | | Handbook of Experimental Pharmacology, pages |
| | | 145-163, Springer-Verlag, Berlin (2008) |
| EWLS | 110 | Newton & Deutscher, Phage Peptide Display in |
| | | Handbook of Experimental Pharmacology, pages |
| | | 145-163, Springer-Verlag, Berlin (2008) |
| SNEW | 111 | Newton & Deutscher, Phage Peptide Display in |
| | | Handbook of Experimental Pharmacology, pages |
| | | 145-163, Springer-Verlag, Berlin (2008) |
| TNYL | 112 | Newton & Deutscher, Phage Peptide Display in |
| | | Handbook of Experimental Pharmacology, pages |
| | | 145-163, Springer-Verlag, Berlin (2008) |
| WIFPWIQL | 113 | Newton & Deutscher, Phage Peptide Display in |
| | | Handbook of Experimental Pharmacology, pages |
| | | 145-163, Springer-Verlag, Berlin (2008) |
| WDLAWMFRLPVG | 114 | Newton & Deutscher, Phage Peptide Display in |
| | | Handbook of Experimental Pharmacology, pages |
| | | 145-163, Springer-Verlag, Berlin (2008) |
| CTVALPGGYVRVC | 115 | Newton & Deutscher, Phage Peptide Display in |
| | | Handbook of Experimental Pharmacology, pages |
| | | 145-163, Springer-Verlag, Berlin (2008) |
| CVPELGHEC | 116 | Newton & Deutscher, Phage Peptide Display in |
| | | Handbook of Experimental Pharmacology, pages |
| | | 145-163, Springer-Verlag, Berlin (2008) |
| CGRRAGGSC | 117 | Newton & Deutscher, Phage Peptide Display in |
| | | Handbook of Experimental Pharmacology, pages |
| | | 145-163, Springer-Verlag, Berlin (2008) |
| CVAYCIEHHCWTC | 118 | Newton & Deutscher, Phage Peptide Display in |
| | | Handbook of Experimental Pharmacology, pages |
| | | 145-163, Springer-Verlag, Berlin (2008) |
| CVFAHNYDYLVC | 119 | Newton & Deutscher, Phage Peptide Display in |
| | | Handbook of Experimental Pharmacology, pages |
| | | 145-163, Springer-Verlag, Berlin (2008) |
| CVFTSNYAFC | 120 | Newton & Deutscher, Phage Peptide Display in |
| | | Handbook of Experimental Pharmacology, pages |
| | | 145-163, Springer-Verlag, Berlin (2008) |
| VHSPNKK | 121 | Newton & Deutscher, Phage Peptide Display in |
| | | Handbook of Experimental Pharmacology, pages |
| | | 145-163, Springer-Verlag, Berlin (2008) |
| CDCRGDCFC | 122 | Newton & Deutscher, Phage Peptide Display in |
| | | Handbook of Experimental Pharmacology, pages |
| | | 145-163, Springer-Verlag, Berlin (2008) |
| CRGDGWC | 123 | Newton & Deutscher, Phage Peptide Display in |
| | | Handbook of Experimental Pharmacology, pages |
| | | 145-163, Springer-Verlag, Berlin (2008) |
| XRGCDX | 124 | Newton & Deutscher, Phage Peptide Display in |
| | | Handbook of Experimental Pharmacology, pages |
| | | 145-163, Springer-Verlag, Berlin (2008) |
| PXX(S/T) | 125 | Newton & Deutscher, Phage Peptide Display in |
| | | Handbook of Experimental Pharmacology, pages |
| | | 145-163, Springer-Verlag, Berlin (2008) |
| CTTHWGFTLC | 126 | Newton & Deutscher, Phage Peptide Display in |
| | | Handbook of Experimental Pharmacology, pages |
| | | 145-163, Springer-Verlag, Berlin (2008) |
| SGKGPRQITAL | 127 | Newton & Deutscher, Phage Peptide Display in |
| | | Handbook of Experimental Pharmacology, pages |
| | | 145-163, Springer-Verlag, Berlin (2008) |
| A(A/Q)(N/A) | 128 | Newton & Deutscher, Phage Peptide Display in |
| (L/Y)(T/V/M/R) | | Handbook of Experimental Pharmacology, pages |
| (R/K) | | 145-163, Springer-Verlag, Berlin (2008) |
| VYMSPF | 129 | Newton & Deutscher, Phage Peptide Display in |
| | | Handbook of Experimental Pharmacology, pages |
| | | 145-163, Springer-Verlag, Berlin (2008) |
| MQLPLAT | 130 | Newton & Deutscher, Phage Peptide Display in |
| | | Handbook of Experimental Pharmacology, pages |
| | | 145-163, Springer-Verlag, Berlin (2008) |
| ATWLPPR | 131 | Newton & Deutscher, Phage Peptide Display in |
| | | Handbook of Experimental Pharmacology, pages |
| | | 145-163, Springer-Verlag, Berlin (2008) |
| HTMYYHHYQHHL | 132 | Newton & Deutscher, Phage Peptide Display in |
| | | Handbook of Experimental Pharmacology, pages |
| | | 145-163, Springer-Verlag, Berlin (2008) |
| SEVGCRAGPLQWLC | 133 | Newton & Deutscher, Phage Peptide Display in |
| EKYFG | | Handbook of Experimental Pharmacology, pages |
| | | 145-163, Springer-Verlag, Berlin (2008) |
| CGLLPVGRPDRNV | 134 | Newton & Deutscher, Phage Peptide Display in |
| WRWLC | | Handbook of Experimental Pharmacology, pages |
| | | 145-163, Springer-Verlag, Berlin (2008) |
| CKGQCDRFKGLPW | 135 | Newton & Deutscher, Phage Peptide Display in |
| EC | | Handbook of Experimental Pharmacology, pages |
| | | 145-163, Springer-Verlag, Berlin (2008) |
| SGRSA | 136 | Newton & Deutscher, Phage Peptide Display in |
| | | Handbook of Experimental Pharmacology, pages |
| | | 145-163, Springer-Verlag, Berlin (2008) |
| WGFP | 137 | Newton & Deutscher, Phage Peptide Display in |
| | | Handbook of Experimental Pharmacology, pages |
| | | 145-163, Springer-Verlag, Berlin (2008) |
| LWXXAr | 138 | Newton & Deutscher, Phage Peptide Display in |
| | | Handbook of Experimental Pharmacology, pages |
| | | 145-163, Springer-Verlag, Berlin (2008) |
| XFXXYLW | 139 | Newton & Deutscher, Phage Peptide Display in |
| | | Handbook of Experimental Pharmacology, pages |
| | | 145-163, Springer-Verlag, Berlin (2008) |
| AEPMPHSLNFSQYL | 140 | Newton & Deutscher, Phage Peptide Display in |
| WYT | | Handbook of Experimental Pharmacology, pages |
| | | 145-163, Springer-Verlag, Berlin (2008) |
| WAY(W/F)SP | 141 | Newton & Deutscher, Phage Peptide Display in |
| | | Handbook of Experimental Pharmacology, pages |
| | | 145-163, Springer-Verlag, Berlin (2008) |
| IELLQAR | 142 | Newton & Deutscher, Phage Peptide Display in |
| | | Handbook of Experimental Pharmacology, pages |
| | | 145-163, Springer-Verlag, Berlin (2008) |
| DITWDQLWDLMK | 143 | Newton & Deutscher, Phage Peptide Display in |
| | | Handbook of Experimental Pharmacology, pages |
| | | 145-163, Springer-Verlag, Berlin (2008) |
| AYTKCSRQWRTCM | 144 | Newton & Deutscher, Phage Peptide Display in |
| TTH | | Handbook of Experimental Pharmacology, pages |
| | | 145-163, Springer-Verlag, Berlin (2008) |
| PQNSKIPGPTFLDP | 145 | Newton & Deutscher, Phage Peptide Display in |
| H | | Handbook of Experimental Pharmacology, pages |
| | | 145-163, Springer-Verlag, Berlin (2008) |
| SMEPALPDWWWK | 146 | Newton & Deutscher, Phage Peptide Display in |
| MFK | | Handbook of Experimental Pharmacology, pages |
| | | 145-163, Springer-Verlag, Berlin (2008) |
| ANTPCGPYTHDCPV | 147 | Newton & Deutscher, Phage Peptide Display in |
| KR | | Handbook of Experimental Pharmacology, pages |
| | | 145-163, Springer-Verlag, Berlin (2008) |
| TACHQHVRMVRP | 148 | Newton & Deutscher, Phage Peptide Display in |
| | | Handbook of Experimental Pharmacology, pages |
| | | 145-163, Springer-Verlag, Berlin (2008) |
| VPWMEPAYQRFL | 149 | Newton & Deutscher, Phage Peptide Display in |
| | | Handbook of Experimental Pharmacology, pages |
| | | 145-163, Springer-Verlag, Berlin (2008) |
| DPRATPGS | 150 | Newton & Deutscher, Phage Peptide Display in |
| | | Handbook of Experimental Pharmacology, pages |
| | | 145-163, Springer-Verlag, Berlin (2008) |
| FRPNRAQDYNTN | 15 | Newton & Deutscher, Phage Peptide Display in |
| | | Handbook of Experimental Pharmacology, pages |
| | | 145-163, Springer-Verlag, Berlin (2008) |
| CTKNSYLMC | 152 | Newton & Deutscher, Phage Peptide Display in |
| | | Handbook of Experimental Pharmacology, pages |
| | | 145-163, Springer-Verlag, Berlin (2008) |
| C(R/Q)L/RT(G/N) | 153 | Newton & Deutscher, Phage Peptide Display in |
| XXG(A/V)GC | | Handbook of Experimental Pharmacology, pages |
| | | 145-163, Springer-Verlag, Berlin (2008) |
| CPIEDRPMC | 154 | Newton & Deutscher, Phage Peptide Display in |
| | | Handbook of Experimental Pharmacology, pages |
| | | 145-163, Springer-Verlag, Berlin (2008) |
| HEWSYLAPYPWF | 155 | Newton & Deutscher, Phage Peptide Display in |
| | | Handbook of Experimental Pharmacology, pages |
| | | 145-163, Springer-Verlag, Berlin (2008) |
| MCPKHPLGC | 156 | Newton & Deutscher, Phage Peptide Display in |
| | | Handbook of Experimental Pharmacology, pages |
| | | 145-163, Springer-Verlag, Berlin (2008) |
| RMWPSSTVNLSAGR | 157 | Newton & Deutscher, Phage Peptide Display in |
| R | | Handbook of Experimental Pharmacology, pages |
| | | 145-163, Springer-Verlag, Berlin (2008) |
| SAKTAVSQRVWLPS | 158 | Newton & Deutscher, Phage Peptide Display in |
| HRGGEP | | Handbook of Experimental Pharmacology, pages |
| | | 145-163, Springer-Verlag, Berlin (2008) |
| KSREHVNNSACPSK | 159 | Newton & Deutscher, Phage Peptide Display in |
| RITAAL | | Handbook of Experimental Pharmacology, pages |
| | | 145-163, Springer-Verlag, Berlin (2008) |
| EGFR | 160 | Newton & Deutscher, Phage Peptide Display in |
| | | Handbook of Experimental Pharmacology, pages |
| | | 145-163, Springer-Verlag, Berlin (2008) |
| AGLGVR | 161 | Newton & Deutscher, Phage Peptide Display in |
| | | Handbook of Experimental Pharmacology, pages |
| | | 145-163, Springer-Verlag, Berlin (2008) |
| GTRQGHTMRLGVS | 162 | Newton & Deutscher, Phage Peptide Display in |
| DG | | Handbook of Experimental Pharmacology, pages |
| | | 145-163, Springer-Verlag, Berlin (2008) |
| IAGLATPGWSHWLA | 163 | Newton & Deutscher, Phage Peptide Display in |
| L | | Handbook of Experimental Pharmacology, pages |
| | | 145-163, Springer-Verlag, Berlin (2008) |
| SMSIARL | 164 | Newton & Deutscher, Phage Peptide Display in |
| | | Handbook of Experimental Pharmacology, pages |
| | | 145-163, Springer-Verlag, Berlin (2008) |
| HTFEPGV | 165 | Newton & Deutscher, Phage Peptide Display in |
| | | Handbook of Experimental Pharmacology, pages |
| | | 145-163, Springer-Verlag, Berlin (2008) |
| NTSLKRISNKR1RR | 166 | Newton & Deutscher, Phage Peptide Display in |
| K | | Handbook of Experimental Pharmacology, pages |
| | | 145-163, Springer-Verlag, Berlin (2008) |
| LRIKRKRRKRKKTR | 167 | Newton & Deutscher, Phage Peptide Display in |
| K | | Handbook of Experimental Pharmacology, pages |
| | | 145-163, Springer-Verlag, Berlin (2008) |

Y* is phospho-Tyr

In some embodiments, the targeting sequence may be a peptide that can be used for chemical coupling (e.g., can comprise arginine and/or lysine residues that can be chemically coupled through their R groups) to another molecule that targets entry into a cell. In some embodiments, the AAV capsid protein or virus capsid of the disclosure can comprise a mutation as described in WO 2006/066066. For example, the capsid protein can comprise a selective amino acid substitution at amino acid position 263, 705, 708 and/or 716 of the native AAV2 capsid protein or a corresponding change(s) in a capsid protein from another AAV serotype.

Additionally, or alternatively, in some embodiments, the capsid protein, virus capsid or vector comprises a selective amino acid insertion directly following amino acid position 264 of the AAV2 capsid protein or a corresponding change in the capsid protein from other AAV. By “directly following amino acid position X” it is intended that the insertion immediately follows the indicated amino acid position (for example, “following amino acid position 264” indicates a point insertion at position 265 or a larger insertion, e.g., from positions 265 to 268, etc.). Furthermore, in some embodiments, the capsid protein, virus capsid or vector of this disclosure can comprise amino acid modifications such as described in PCT Publication No. WO 2010/093784 (e.g., 2i8) and/or in PCT Publication No. WO 2014/144229 (e.g., dual glycan).

Heterologous molecules are defined as those that are not naturally found in an AAV infection, e.g., those not encoded by a wild-type AAV genome. Further, therapeutically useful molecules can be associated with the outside of the chimeric virus capsid for transfer of the molecules into host target cells. Such associated molecules can include DNA, RNA, small organic molecules, metals, carbohydrates, lipids and/or polypeptides. In one embodiment of the disclosure the therapeutically useful molecule is covalently linked (i.e., conjugated or chemically coupled) to the capsid proteins. Methods of covalently linking molecules are known by those skilled in the art.

d. Modified Viral Vectors

The disclosure provides virus vectors comprising the capsid protein variants and capsids of the disclosure. In some embodiments, the virus vector is a parvovirus vector (e.g., comprising a parvovirus capsid and/or vector genome), for example, an AAV vector (e.g., comprising an AAV capsid and/or vector genome). In some embodiments, the virus vector comprises a modified AAV capsid comprising a modified capsid protein of the disclosure and a vector genome.

For example, in some embodiments, the virus vector comprises: (a) a virus capsid (e.g., an AAV capsid) comprising a capsid protein variant of the disclosure; and (b) a nucleic acid comprising a terminal repeat sequence (e.g., an AAV TR), wherein the nucleic acid comprising the terminal repeat sequence is encapsidated by the virus capsid. The nucleic acid can optionally comprise two terminal repeats (e.g., two AAV TRs). In representative embodiments, the virus vector is a recombinant virus vector comprising a heterologous nucleic acid encoding a polypeptide or functional RNA of interest.

AAVs do not typically transduce T-cells at high levels. In contrast, in some embodiments, the virus vectors of the disclosure exhibit enhanced transduction of one or more cell types (e.g., T-cells) and/or tissues, as compared with the level of transduction by a wild type virus vector, or a virus vector without the capsid protein variant. In some embodiments, an AAV viral vector has increased cellular transduction compared to a wild type or native AAV viral vector. In some embodiments, the AAV viral vector has increased transduction in one or more cell types (e.g., T-cells) compared to a wild type or native AAV viral vector, or an AAV viral vector that does not comprise any one of the capsid protein variants disclosed herein. In some embodiments, the AAV viral vector may have increased transduction into a hematopoietic stem cell. In some embodiments, the AAV viral vector may have increased transduction in monocytes, basophils, eosinophils, neutrophils, dendritic cells, macrophages, B-cells, T-cells, and/or natural killer cells. In some embodiments, the AAV viral vector may have increased transduction in satellite cells, mesenchymal stem cells, and/or basal cells. In some embodiments, the AAV viral vector may have increased transduction in lung epithelial cells, hepatocytes, and/or skeletal muscle cells.

Known receptors and co-receptors for AAVs include heparan sulfate proteoglycans, integrins, O-linked sialic acid, N-linked sialic acid, AAV receptor (AAVR, KIAA0319L), hepatocyte growth factor receptor (c-Met), CD9, FGFR-1, 37/67-kDa laminin receptor, and platelet derived growth factor receptor. In embodiments, the AAV viral vectors of the disclosure have increased affinity for one or more of these receptors and/or co-receptors. For example, in some embodiments, the AAV viral vector has increased heparin and/or heparan sulfate binding compared to a wildtype or native AAV viral vector. In some embodiments, the AAV viral vector has increased sialic acid binding compared to a wildtype or native AAV viral vector. In some embodiments, the AAV viral vector has increased integrin binding compared to wildtype or native AAV viral vector. In some embodiments, the AAV viral vector has increased binding to an integrin that comprises an α subunit and a β subunit, compared to wildtype or native AAV viral vector. The integrin may be, for example, α4β7, α4β1, α1β1, α2β1, αEβ7, αLβ2, α5β1, α5β6, α5β5, α5β8, α5β8, α3β1, α5β1, α11β1, α5β3, α11β3, αVβ3, αVβ5, αVβ6, αVβ8.

The disclosure also provides a nucleotide sequence, or an expression vector comprising the same, that encodes one or more of the capsid protein variants (e.g. AAV capsid protein variants) of the disclosure or one or more the capsids (e.g. AAV capsids) comprising a capsid protein variant. In some embodiments, the nucleic acids encode a recombinant AAV capsid protein having the sequence of any one of SEQ ID NOs: 2, 4, 6, 8, 10, 12, and 14. In some embodiments, the nucleic acid comprises a sequence selected from the group consisting of SEQ ID NOs: 3, 5, 7, 9, 11, 13, and 15. The nucleotide sequence may be a DNA sequence or an RNA sequence. The expression vector is not limited and may be a viral vector (e.g., adenovirus, AAV, herpesvirus, vaccinia, poxviruses, baculoviruses, and the like), or a non-viral vector such as plasmids, phage, YACs, BACs, and the like. The present disclosure also provides a cell that comprises one or more nucleotide sequences or expression vectors of the disclosure. The cells may be in vitro, ex vivo, or in vivo.

### Methods for Producing Virus Vectors

The present disclosure further provides methods of producing the virus vectors disclosed herein. Thus, in some embodiments, the present disclosure provides a method of producing an AAV vector that has increased cellular transduction (e.g., increased transduction into T-cells), comprising: a) identifying surface-exposed residues on an AAV capsid protein; b) generating a library of AAV capsid proteins comprising amino acid substitutions of the surface-exposed amino acid residues identified in (a); c) producing AAV particles comprising capsid proteins from the library of AAV capsid proteins of (b); d) contacting the AAV particles of (c) with cells under conditions whereby infection and replication can occur; e) selecting AAV particles that can complete at least one infectious cycle and replicate to titers similar to or greater than control AAV particles. In some embodiments, steps (d) and (e) are repeated more than one time, for example 2, 3, 4, 5, 6, 7, 8, 9, or 10 times. Non-limiting examples of methods for identifying surface-exposed residues include cryo-electron microscopy. See also, description of the crystal structure of AAV2 (Xie et al., (2002) Proc. Nat. Acad. Sci. 99:10405-10), AAV9 (DiMattia et al., (2012) J. Virol. 86:6947-6958), AAV8 (Nam et al, (2007) J. Virol. 81:12260-12271), AAV6 (Ng et al., (2010) J. Virol. 84:12945-12957), AAV5 (Govindasamy et al. (2013) J. Virol. 87, 11187-11199), AAV4 (Govindasamy et al. (2006) J. Virol. 80:11556-11570), AAV3B (Lerch et al., (2010) Virology 403:26-36), BPV (Kailasan et al., (2015) J. Virol. 89:2603-2614) and CPV (Xie et al, (1996) J. Mol. Biol. 6:497-520 and Tsao et al, (1991) Science 251:1456-64).

Resolution and identification of the surface-exposed residues allows for their subsequent modification through random, rational and/or degenerate mutagenesis to generate AAV capsids that can be identified through further selection and/or screening. Thus, in a further embodiment, the present disclosure provides a method of producing an AAV vector that has increased cellular transduction (e.g., increased transduction into T-cells), comprising: a) identifying surface-exposed amino acid residues on an AAV capsid protein; b) generating AAV capsid proteins comprising amino acid substitutions of the surface-exposed amino acid residues identified in (a) by random, rational and/or degenerate mutagenesis; c) producing AAV particles comprising capsid proteins from the AAV capsid proteins of (b); d) contacting the AAV particles of (c) with cells under conditions whereby infection and replication can occur; and e) selecting AAV particles that can complete at least one infectious cycle and replicate to titers similar to or greater than control AAV particles.

Methods of generating AAV capsid proteins comprising amino acid substitutions of surface-exposed amino acid residues by random, rational and/or degenerate mutagenesis are known in the art. This comprehensive approach presents a platform technology that can be applied to modifying any AAV capsid. Application of this platform technology yields AAV variants derived from the original AAV capsid template that have enhanced transduction efficiency. As one advantage and benefit, application of this technology will expand the cohort of patients eligible for gene therapy with AAV vectors.

In some embodiments, the present disclosure provides a method of producing a virus vector, the method comprising providing to a cell: (a) a nucleic acid template comprising at least one TR sequence (e.g., AAV TR sequence), and (b) AAV sequences sufficient for replication of the nucleic acid template and encapsidation into AAV capsids (e.g., AAV rep sequences and AAV cap sequences encoding the AAV capsids of the disclosure). Optionally, the nucleic acid template further comprises at least one heterologous nucleic acid sequence. In some embodiments, the nucleic acid template comprises two AAV ITR sequences, which are located 5′ and 3′ to the heterologous nucleic acid sequence (if present), although they need not be directly contiguous thereto.

The nucleic acid template and AAV rep and cap sequences are provided under conditions such that virus vector comprising the nucleic acid template packaged within the AAV capsid is produced in the cell. The method can further comprise the step of collecting the virus vector from the cell. The virus vector can be collected from the medium and/or by lysing the cells. The cell can be a cell that is permissive for AAV viral replication. Any suitable cell known in the art may be employed. In some embodiments, the cell is a mammalian cell. In some embodiments, the cell can be a trans-complementing packaging cell line that provides functions deleted from a replication-defective helper virus, e.g., 293 cells or other E1a trans-complementing cells.

The AAV replication and capsid sequences may be provided by any method known in the art. Current protocols typically express the AAV rep/cap genes on a single plasmid. The AAV replication and packaging sequences need not be provided together, although it may be convenient to do so. The AAV rep and/or cap sequences may be provided by any viral or non-viral vector. For example, the rep/cap sequences may be provided by a hybrid adenovirus or herpesvirus vector (e.g., inserted into the E1a or E3 regions of a deleted adenovirus vector). EBV vectors may also be employed to express the AAV cap and rep genes. One advantage of this method is that EBV vectors are episomal, yet will maintain a high copy number throughout successive cell divisions (i.e., are stably integrated into the cell as extra-chromosomal elements, designated as an “EBV based nuclear episome,” see Margolski, (1992) Curr. Top. Microbiol. Immun. 158:67). As a further alternative, the rep/cap sequences may be stably incorporated into a cell. Typically the AAV rep/cap sequences will not be flanked by the TRs, to prevent rescue and/or packaging of these sequences.

The nucleic acid template can be provided to the cell using any method known in the art. For example, the template can be supplied by a non-viral (e.g., plasmid) or viral vector. In some embodiments, the nucleic acid template is supplied by a herpesvirus or adenovirus vector (e.g., inserted into the E1a or E3 regions of a deleted adenovirus). As another illustration, Palombo et al., (1998) J. Virology 72:5025, describes a baculovirus vector carrying a reporter gene flanked by the AAV TRs. EBV vectors may also be employed to deliver the template, as described above with respect to the rep/cap genes.

In some embodiments, the nucleic acid template is provided by a replicating rAAV virus. In some embodiments, an AAV provirus comprising the nucleic acid template is stably integrated into the chromosome of the cell. To enhance virus titers, helper virus functions (e.g., adenovirus or herpesvirus) that promote a productive AAV infection can be provided to the cell. Helper virus sequences necessary for AAV replication are known in the art. Typically, these sequences will be provided by a helper adenovirus or herpesvirus vector. Alternatively, the adenovirus or herpesvirus sequences can be provided by another non-viral or viral vector, e.g., as a noninfectious adenovirus miniplasmid that carries all of the helper genes that promote efficient AAV production as described by Ferrari et al., (1997) Nature Med. 3:1295, and U.S. Pat. Nos. 6,040,183 and 6,093,570.

Further, the helper virus functions may be provided by a packaging cell with the helper sequences embedded in the chromosome or maintained as a stable extrachromosomal element. Generally, the helper virus sequences cannot be packaged into AAV virions, e.g., are not flanked by TRs. Those skilled in the art will appreciate that it may be advantageous to provide the

AAV replication and capsid sequences and the helper virus sequences (e.g., adenovirus sequences) on a single helper construct. This helper construct may be a non-viral or viral construct. As one nonlimiting illustration, the helper construct can be a hybrid adenovirus or hybrid herpesvirus comprising the AAV rep/cap genes. In some embodiments, the AAV rep/cap sequences and the adenovirus helper sequences are supplied by a single adenovirus helper vector. This vector further can further comprise the nucleic acid template. The AAV rep/cap sequences and/or the rAAV template can be inserted into a deleted region (e.g., the E1a or E3 regions) of the adenovirus.

In some embodiments, the AAV rep/cap sequences and the adenovirus helper sequences are supplied by a single adenovirus helper vector. The rAAV template can be provided, for example, as a plasmid template. In some embodiments, the AAV rep/cap sequences and adenovirus helper sequences are provided by a single adenovirus helper vector, and the rAAV template is integrated into the cell as a provirus. Alternatively, the rAAV template is provided by an EBV vector that is maintained within the cell as an extrachromosomal element (e.g., as an EBV based nuclear episome).

In some embodiments, the AAV rep/cap sequences and adenovirus helper sequences are provided by a single adenovirus helper. The rAAV template can be provided as a separate replicating viral vector. For example, the rAAV template can be provided by a rAAV particle or a second recombinant adenovirus particle. According to the foregoing methods, the hybrid adenovirus vector typically comprises the adenovirus 5′ and 3′ cis sequences sufficient for adenovirus replication and packaging (i.e., the adenovirus terminal repeats and PAC sequence). The AAV rep/cap sequences and, if present, the rAAV template are embedded in the adenovirus backbone and are flanked by the 5′ and 3′ cis sequences, so that these sequences may be packaged into adenovirus capsids. As described above, the adenovirus helper sequences and the AAV rep/cap sequences are generally not flanked by TRs so that these sequences are not packaged into the AAV virions. Zhang et al., ((2001) Gene Ther. 18:704-12) describe a chimeric helper comprising both adenovirus and the AAV rep and cap genes.

Herpesvirus may also be used as a helper virus in AAV packaging methods. Hybrid herpesviruses encoding the AAV Rep protein(s) may advantageously facilitate scalable AAV vector production schemes. A hybrid herpes simplex virus type I (HSV-1) vector expressing the AAV-2 rep and cap genes has been described (Conway et al., (1999) Gene Therapy 6:986 and WO 00/17377. In some embodiments, the virus vectors of the disclosure can be produced in insect cells using baculovirus vectors to deliver the rep/cap genes and rAAV template as described, for example, by Urabe et al., (2002) Human Gene Therapy 13: 1935-43.

AAV vector stocks free of contaminating helper virus may be obtained by any method known in the art. For example, AAV and helper virus may be readily differentiated based on size. AAV may also be separated away from helper virus based on affinity for a heparan substrate (Zolotukhin et al. (1999) Gene Therapy 6:973). Deleted replication-defective helper viruses can be used so that any contaminating helper virus is not replication competent. In some embodiments, an adenovirus helper lacking late gene expression may be employed, as only adenovirus early gene expression is required to mediate packaging of AAV virus. Adenovirus mutants defective for late gene expression are known in the art (e.g., ts100K and ts149 adenovirus mutants).

### Recombinant Virus Vectors

The disclosure provides recombinant viral vectors (e.g. recombinant AAV vectors) comprising at least one of the capsid proteins (e.g. AAV capsid proteins) or at least one of the capsids (e.g. AAV capsids) disclosed herein, wherein the capsid protein comprises one or more transduction-associated peptides disclosed herein. In some embodiments, the AAV vector exhibits increased transduction into a cell, such as a T-cell, compared to a wild type AAV vector or an AAV vector that does not comprise the transduction-associated peptide. In some embodiments, the AAV vector exhibits increased transduction into the nucleus of a T-cell as compared to a wild type AAV vector or an AAV vector that does not comprise the transduction-associated peptide. In some embodiments, the AAV vector exhibits increased transduction into the cytosol of a T-cell as compared to a wild type AAV vector or an AAV vector that does not comprise the transduction-associated peptide.

The recombinant virus vectors of the present disclosure are useful for the delivery of nucleic acids to cells in vitro, ex vivo, and in vivo. Molecules that can be packaged by the modified virus capsid and transferred into a cell include heterologous DNA, RNA, polypeptides, small organic molecules, metals, or combinations of the same. In particular, the virus vectors can be advantageously employed to deliver or transfer nucleic acids to animal cells, including mammalian cells. Thus, in some embodiments, a nucleic acid (“cargo nucleic acid”) may be encapsidated by a capsid protein of the disclosure. The cargo nucleic acid sequence delivered in the virus vectors of the present disclosure may be any heterologous nucleic acid sequence(s) of interest.

In some embodiments, the expression of the heterologous nucleic acid delivered by the AAV vectors disclosed herein is increased as compared to the expression of the heterologous nucleic acid delivered by a wild type AAV vector (such as, AAV6 vector), or an AAV vector that does not comprise the transduction-associated peptide disclosed herein. In some embodiments, the expression of the heterologous nucleic acid delivered by the AAV vectors disclosed herein is increased at least about 1.5 fold, for example about 2 fold, 2.5 fold, 3 fold, 3.5 fold, 4, fold, 4.5 fold, 5 fold, 6 fold, 7 fold, 8 fold, 9 fold, or 10 fold, including all values and subranges that lie therebetween, as compared to the expression of the heterologous nucleic acid delivered by a wild type AAV vector (such as, AAV6 vector), or an AAV vector that does not comprise the transduction-associated peptide disclosed herein. In some embodiments, the expression of the heterologous nucleic acid delivered by the AAV vectors disclosed herein is increased at least about 10%, for example, about 20%, about 30%, about 40%, about 50%, about 60%, about 70%, about 80%, about 90%, or about 100%, including all values and subranges that lie therebetween, as compared to the expression of the heterologous nucleic acid delivered by a wild type AAV vector (such as, AAV6 vector), or an AAV vector that does not comprise the transduction-associated peptide disclosed herein.

Nucleic acids of interest include nucleic acids encoding polypeptides, including therapeutic (e.g., for medical or veterinary uses) or immunogenic (e.g., for vaccines) polypeptides or RNAs. In some embodiments, the cargo nucleic acid encodes a therapeutic protein or a therapeutic RNA.

Therapeutic polypeptides may include, but are not limited to, a chimeric antigen receptor (CAR), ABCD1, beta globin (HBB), hemoglobin A, hemoglobin F, cystic fibrosis transmembrane regulator protein (CFTR), dystrophin (including mini- and micro-dystrophins, see, e.g., Vincent et al, (1993) Nature Genetics 5:130; U.S. Patent Publication No. 2003/017131; International publication WO/2008/088895, Wang et al., Proc. Natl. Acad. Sci. USA 97:1 3714-13719 (2000); and Gregorevic et al., Mol. Ther. 16:657-64 (2008)), myostatin propeptide, follistatin, activin type 11 soluble receptor, IGF-1, anti-inflammatory polypeptides such as the Ikappa B dominant mutant, sarcospan, utrophin (Tinsley et al, (1996) Nature 384:349), mini-utrophin, clotting factors (e.g., Factor VIII, Factor IX, Factor X, etc.), erythropoietin, angiostatin, endostatin, catalase, tyrosine hydroxylase, superoxide dismutase, leptin, the LDL receptor, lipoprotein lipase, ornithine transcarbamylase, β-globin, α-globin, spectrin, alpha-1-antitrypsin, adenosine deaminase, hypoxanthine guanine phosphoribosyl transferase, β-glucocerebrosidase, sphingomyelinase, lysosomal hexosaminidase A, branched-chain keto acid dehydrogenase, RP65 protein, cytokines (e.g., alpha-interferon, beta-interferon, gamma-interferon, interleukin-2, interleukin-4, granulocyte-macrophage colony stimulating factor, lymphotoxin, and the like), peptide growth factors, neurotrophic factors and hormones (e.g., somatotropin, insulin, insulin-like growth factors 1 and 2, platelet derived growth factor, epidermal growth factor, fibroblast growth factor, nerve growth factor, neurotrophic factor −3 and −4, brain-derived neurotrophic factor, bone morphogenic proteins [including RANKL and VEGF], glial derived growth factor, transforming growth factor −α and −β, and the like), lysosomal acid alpha-glucosidase, alpha-galactosidase A, receptors (e.g., the tumor necrosis growth factor soluble receptor), S100A1, parvalbumin, adenylyl cyclase type 6, a molecule that modulates calcium handling (e.g., SERCA_{2A}, Inhibitor 1 of PP1 and fragments thereof [e.g., WO 2006/029319 and WO 2007/100465]), a molecule that effects G-protein coupled receptor kinase type 2 knockdown such as a truncated constitutively active bARKct, anti-inflammatory factors such as TRAP, anti-myostatin proteins, aspartoacylase, monoclonal antibodies (including single chain monoclonal antibodies; an exemplary Mab is the Herceptin® Mab), neuropeptides and fragments thereof (e.g., galanin, Neuropeptide Y (see, U.S. Pat. No. 7,071,172), angiogenesis inhibitors such as Vasohibins and other VEGF inhibitors (e.g., Vasohibin 2 [see, WO JP2006/073052]). Other illustrative heterologous nucleic acid sequences encode suicide gene products (e.g., thymidine kinase, cytosine deaminase, diphtheria toxin, and tumor necrosis factor), proteins that enhance or inhibit transcription of host factors (e.g., nuclease-dead Cas9 linked to a transcription enhancer or inhibitor element, zinc-finger proteins linked to a transcription enhancer or inhibitor element, transcription activator-like (TAL) effectors linked to a transcription enhancer or inhibitor element), proteins conferring resistance to a drug used in cancer therapy, tumor suppressor gene products (e.g., p53, Rb, Wt-1), TRAIL, FAS-ligand, and any other polypeptide that has a therapeutic effect in a subject in need thereof. AAV vectors can also be used to deliver monoclonal antibodies and antibody fragments, for example, an antibody or antibody fragment directed against myostatin (see, e.g., Fang et al., Nature Biotechnology 23:584-590 (2005)). Heterologous nucleic acid sequences encoding polypeptides include those encoding reporter polypeptides (e.g., an enzyme). Reporter polypeptides are known in the art and include, but are not limited to, Green Fluorescent Protein, β-galactosidase, alkaline phosphatase, luciferase, and chloramphenicol acetyltransferase gene. Optionally, the heterologous nucleic acid encodes a secreted polypeptide (e.g., a polypeptide that is a secreted polypeptide in its native state or that has been engineered to be secreted, for example, by operable association with a secretory signal sequence as is known in the art).

Alternatively, in some embodiments of this disclosure, the heterologous nucleic acid may encode an antisense nucleic acid, a ribozyme (e.g., as described in U.S. Pat. No. 5,877,022), RNAs that effect spliceosome-mediated/ram-splicing (see, Puttaraju et al, (1999) Nature Biotech. 17:246; U.S. Pat. Nos. 6,013,487; 6,083,702), interfering RNAs (RNAi) including siRNA, shRNA or miRNA that mediate gene silencing (see, Sharp et al, (2000) Science 287:2431), and other non-translated RNAs, such as “guide” RNAs (Gorman et al., (1998) Proc. Nat. Acad. Sci. USA 95:4929; U.S. Pat. No. 5,869,248 to Yuan et al.), and the like. Exemplary untranslated RNAs include RNAi against a multiple drug resistance (MDR) gene product (e.g., to treat and/or prevent tumors and/or for administration to the heart to prevent damage by chemotherapy), RNAi against myostatin (e.g., for Duchenne muscular dystrophy), RNAi against VEGF (e.g., to treat and/or prevent tumors), RNAi against phospholamban (e.g., to treat cardiovascular disease, see, e.g., Andino et al., J. Gene Med. 10:132-142 (2008) and Li et al., Acta Pharmacol Sin. 26:51-55 (2005)); phospholamban inhibitory or dominant-negative molecules such as phospholamban S 16E (e.g., to treat cardiovascular disease, see, e.g., Hoshijima et al. Nat. Med. 8:864-871 (2002)), RNAi to adenosine kinase (e.g., for epilepsy), and RNAi directed against pathogenic organisms and viruses (e.g., hepatitis B and/or C virus, human immunodeficiency virus, CMV, herpes simplex virus, human papilloma virus, etc.).

Further, a nucleic acid sequence that directs alternative splicing can be delivered. To illustrate, an antisense sequence (or other inhibitory sequence) complementary to the 5′ and/or 3′ splice site of dystrophin exon 51 can be delivered in conjunction with a U1 or U7 small nuclear (sn) RNA promoter to induce skipping of this exon. For example, a DNA sequence comprising a U1 or U7 snRNA promoter located 5′ to the antisense/inhibitory sequence(s) can be packaged and delivered in a modified capsid of the disclosure.

In some embodiments, a nucleic acid sequence that directs gene editing can be delivered. For example, the nucleic acid may encode a gene-editing molecule such as a guide RNA or a nuclease. In some embodiments, the nucleic acid may encode a zinc-finger nuclease, a homing endonuclease, a TALEN (transcription activator-like effector nuclease), a NgAgo (agronaute endonuclease), a SGN (structure-guided endonuclease), or a RGN (RNA-guided nuclease) such as a Cas9 nuclease or a Cpf1 nuclease.

The virus vector may also comprise a heterologous nucleic acid that shares homology with and recombines with a locus on a host chromosome. This approach can be utilized, for example, to correct a genetic defect in the host cell.

The present disclosure also provides virus vectors that express an immunogenic polypeptide, e.g., for vaccination. The nucleic acid may encode any immunogen of interest known in the art including, but not limited to, immunogens from human immunodeficiency virus (HIV), simian immunodeficiency virus (SIV), influenza virus, HIV or SIV gag proteins, tumor antigens, cancer antigens, bacterial antigens, viral antigens, and the like.

The use of parvoviruses as vaccine vectors is known in the art (see, e.g., Miyamura el al, (1994) Proc. Nat. Acad. Sci USA 91:8507; U.S. Pat. No. 5,916,563 to Young et al, U.S. Pat. No. 5,905,040 to Mazzara et al, U.S. Pat. Nos. 5,882,652, 5,863,541 to Samulski et al). The antigen may be presented in the parvovirus capsid. Alternatively, the antigen may be expressed from a heterologous nucleic acid introduced into a recombinant vector genome. Any immunogen of interest as described herein and/or as is known in the art can be provided by the virus vector of the present disclosure.

An immunogenic polypeptide can be any polypeptide suitable for eliciting an immune response and/or protecting the subject against an infection and/or disease, including, but not limited to, microbial, bacterial, protozoal, parasitic, fungal and/or viral infections and diseases. For example, the immunogenic polypeptide can be an orthomyxovirus immunogen (e.g., an influenza virus immunogen, such as the influenza virus hemagglutinin (HA) surface protein or the influenza virus nucleoprotein, or an equine influenza virus immunogen) or a lentivirus immunogen (e.g., an equine infectious anemia virus immunogen, a Simian Immunodeficiency Virus (SIV) immunogen, or a Human Immunodeficiency Virus (HIV) immunogen, such as the HIV or SIV envelope GP 160 protein, the HIV or SIV matrix/capsid proteins, and the HIV or SIV gag, pol and env genes products). The immunogenic polypeptide can also be an arenavirus immunogen (e.g., Lassa fever virus immunogen, such as the Lassa fever virus nucleocapsid protein and the Lassa fever envelope glycoprotein), a poxvirus immunogen (e.g., a vaccinia virus immunogen, such as the vaccinia LI or L8 gene products), a flavivirus immunogen (e.g., a yellow fever virus immunogen or a Japanese encephalitis virus immunogen), a filovirus immunogen (e.g., an Ebola virus immunogen, or a Marburg virus immunogen, such as NP and GP gene products), a bunyavirus immunogen (e.g., RVFV, CCHF, and/or SFS virus immunogens), or a coronavirus immunogen (e.g., an infectious human coronavirus immunogen, such as the human coronavirus envelope glycoprotein, or a porcine transmissible gastroenteritis virus immunogen, or an avian infectious bronchitis virus immunogen). The immunogenic polypeptide can further be a polio immunogen, a herpes immunogen (e.g., CMV, EBV, HSV immunogens), a mumps immunogen, a measles immunogen, a rubella immunogen, a diphtheria toxin or other diphtheria immunogen, a pertussis antigen, a hepatitis (e.g., hepatitis A, hepatitis B, hepatitis C, etc.) immunogen, and/or any other vaccine immunogen now known in the art or later identified as an immunogen.

Alternatively, the immunogenic polypeptide can be any tumor or cancer cell antigen. Optionally, the tumor or cancer antigen is expressed on the surface of the cancer cell.

Exemplary cancer and tumor cell antigens are described in S.A. Rosenberg (Immunity 10:281 (1991)). Other illustrative cancer and tumor antigens include, but are not limited to: BRCA1 gene product, BRCA2 gene product, gp100, tyrosinase, GAGE-1/2, BAGE, RAGE, LAGE, NY-ESO-1, CDK-4, β-catenin, MUM-1, Caspase-8, KIAA0205, HPVE, SART-1, FRAME, p15, melanoma tumor antigens (Kawakami et al., (1994) Proc. Natl. Acad. Sci. USA 91:3515; Kawakami et al., (1994) J. Exp. Med., 180:347; Kawakami et al., (1994) Cancer Res. 54:3124), MART-1, gp100, MAGE-1, MAGE-2, MAGE-3, CEA, TRP-1, TRP-2, P-15, tyrosinase (Brichard et al., (1993) J Exp. Med. 178:489); HER-2/neu gene product (U.S. Pat. No. 4.968.603), CA 125, LK26, FB5 (endosialin), TAG 72, AFP, CA 19-9, NSE, DU-PAN-2, CA50, SPan-1, CA72-4, HCG, STN (sialyl Tn antigen), c-erbB-2 proteins, PSA, L-CanAg, estrogen receptor, milk fat globulin, p53 tumor suppressor protein (Levine, (1993) Ann. Rev. Biochem. 62:623); mucin antigens (International Patent Publication No. WO 90/05142); telomerases; nuclear matrix proteins; prostatic acid phosphatase; papilloma virus antigens; and/or antigens now known or later discovered to be associated with the following cancers: melanoma, adenocarcinoma, thymoma, lymphoma (e.g., non-Hodgkin's lymphoma, Hodgkin's lymphoma), sarcoma, lung cancer, liver cancer, colon cancer, leukemia, uterine cancer, breast cancer, prostate cancer, ovarian cancer, cervical cancer, bladder cancer, kidney cancer, pancreatic cancer, brain cancer and any other cancer or malignant condition or metastasis thereof now known or later identified (see, e.g., Rosenberg, (1996) Ann. Rev. Med. 47:481-91).

It will be understood by those skilled in the art that the heterologous nucleic acid(s) of interest can be operably associated with appropriate control sequences. For example, the heterologous nucleic acid can be operably associated with expression control elements, such as transcription/translation control signals, origins of replication, polyadenylation signals, internal ribosome entry sites (IRES), promoters, and/or enhancers, and the like.

Further, regulated expression of the heterologous nucleic acid(s) of interest can be achieved at the post-transcriptional level, e.g., by regulating selective splicing of different introns by the presence or absence of an oligonucleotide, small molecule and/or other compound that selectively blocks splicing activity at specific sites (e.g., as described in WO 2006/119137).

Those skilled in the art will appreciate that a variety of promoter/enhancer elements can be used depending on the level and tissue-specific expression desired. The promoter/enhancer can be constitutive or inducible, depending on the pattern of expression desired. The promoter/enhancer can be native or foreign and can be a natural or a synthetic sequence. By foreign, it is intended that the transcriptional initiation region is not found in the wild-type host into which the transcriptional initiation region is introduced.

In some embodiments, the promoter/enhancer elements can be native to the target cell or subject to be treated. In representative embodiments, the promoters/enhancer element can be native to the heterologous nucleic acid sequence. The promoter/enhancer element is generally chosen so that it functions in the target cell(s) of interest. Further, in some embodiments the promoter/enhancer element is a mammalian promoter/enhancer element. The promoter/enhancer element may be constitutive or inducible.

Inducible expression control elements are typically advantageous in those applications in which it is desirable to provide regulation over expression of the heterologous nucleic acid sequence(s). Inducible promoters/enhancer elements for gene delivery can be tissue-specific or -preferred promoter/enhancer elements, and include muscle specific or preferred (including cardiac, skeletal and/or smooth muscle specific or preferred), neural tissue specific or preferred (including brain-specific or preferred), eye specific or preferred (including retina-specific and cornea-specific), liver specific or preferred, bone marrow specific or preferred, pancreatic specific or preferred, spleen specific or preferred, and lung specific or preferred promoter/enhancer elements. In some embodiments, the inducible expression control elements, such as promoters and/or enhancers, promote selective expression in T-cells. Other inducible promoter/enhancer elements include hormone-inducible and metal-inducible elements. Exemplary inducible promoters/enhancer elements include, but are not limited to, a Tet on/off element, a RU486-inducible promoter, an ecdysone-inducible promoter, a rapamycin-inducible promoter, and a metallothionein promoter.

In embodiments wherein the heterologous nucleic acid sequence(s) is transcribed and then translated in the target cells, specific initiation signals are generally included for efficient translation of inserted protein coding sequences. These exogenous translational control sequences, which may include the ATG initiation codon and adjacent sequences, can be of a variety of origins, both natural and synthetic.

### Pharmaceutical Compositions and Methods of Use

The disclosure also provides compositions comprising at least one of the AAV capsid proteins, the AAV capsids, the viral vectors, the nucleic acids, the expression vectors and/or the cells disclosed herein. In some embodiments, the compositions further comprise a pharmaceutically acceptable carrier. In some embodiments, a pharmaceutical composition is provided comprising a virus vector and/or capsid and/or capsid protein and/or virus particle of the disclosure in a pharmaceutically acceptable carrier and, optionally, other medicinal agents, pharmaceutical agents, stabilizing agents, buffers, carriers, adjuvants, diluents, etc. For injection, the carrier will typically be a liquid. For other modes of administration, the carrier may be either solid or liquid. For inhalation administration, the carrier will be respirable, and optionally can be in solid or liquid particulate form. By “pharmaceutically acceptable” it is meant a material that is not toxic or otherwise undesirable, i.e., the material may be administered to a subject without causing any undesirable biological effects.

The virus vectors according to the present disclosure provide a means for delivering heterologous nucleic acids into a broad range of cells, including dividing and non-dividing cells. In some embodiments, the cell is a T-cell. The virus vectors can be employed to deliver a nucleic acid of interest to a cell in vitro, e.g., to produce a polypeptide in vitro or for ex vivo gene therapy. The virus vectors are additionally useful in a method of delivering a nucleic acid to a subject in need thereof e.g., to express an immunogenic or therapeutic polypeptide or a functional RNA. In this manner, the polypeptide or functional RNA can be produced in vivo in the subject. The subject can be in need of the polypeptide because the subject has a deficiency of the polypeptide. Further, the method can be practiced because the production of the polypeptide or functional RNA in the subject may impart some beneficial effect. In some embodiments, the methods comprise expressing the polypeptide or RNA in the cell in vitro, ex vivo or in vivo, and optionally, isolating the polypeptide or RNA from the cell. The virus vectors can also be used to produce a polypeptide of interest or functional RNA in cultured cells or in a subject (e.g., using the subject as a bioreactor to produce the polypeptide or to observe the effects of the functional RNA on the subject, for example, in connection with screening methods).

The disclosure provides methods of administering any one of the virus vectors, virus particles and/or compositions of this disclosure to a subject. Therefore, the disclosure provides methods of treating a subject in need thereof, comprising administering to the subject an effective amount of any one of the viral vectors (e.g. AAV vectors), any one of the viral particles (e.g. AAV particles), and/or any one of the compositions disclosed herein. Accordingly, the disclosure provides any one of the viral vectors (e.g. AAV vectors), any one of the viral particles (e.g. AAV particles), and/or any one of the compositions disclosed herein for use as a medicament, and/or for use in a method of treatment of a subject in need thereof.

In some embodiments, the virus vectors of the present disclosure can be employed to deliver a heterologous nucleic acid encoding a polypeptide or functional RNA to treat and/or prevent any disease state for which it is beneficial to deliver a therapeutic polypeptide or functional RNA. In some embodiments, the disease state is associated with, correlated with or caused by a dysfunction in, or increase in T-cells. In some embodiments, the disease states include, but are not limited to: cystic fibrosis (cystic fibrosis transmembrane regulator protein) and other diseases of the lung, hemophilia A (Factor VIII), hemophilia B (Factor IX), thalassemia (β-globin), anemia (erythropoietin) and other blood disorders. Alzheimer's disease (GDF; neprilysin), multiple sclerosis (β-interferon), Parkinson's disease (glial-cell line derived neurotrophic factor [GDNF]), Huntington's disease (RNAi to remove repeats), amyotrophic lateral sclerosis, epilepsy (galanin, neurotrophic factors), and other neurological disorders, cancer (endostatin, angiostatin, TRAIL, FAS-ligand, cytokines including interferons; RNAi including RNAi against VEGF or the multiple drug resistance gene product, mir-26a [e.g., for hepatocellular carcinoma]), diabetes mellitus (insulin), muscular dystrophies including Duchenne (dystrophin, mini-dystrophin, insulin-like growth factor I, a sarcoglycan [e.g., a, β, γ], RNAi against myostatic myostatin propeptide, follistatin, activin type II soluble receptor, anti-inflammatory polypeptides such as the Ikappa B dominant mutant, sarcospan, utrophin, mini-utrophin, antisense or RNAi against splice junctions in the dystrophin gene to induce exon skipping [see, e.g., WO/2003/095647], antisense against U7 snRNAs to induce exon skipping [see, e.g., WO/2006/021724], and antibodies or antibody fragments against myostatin or myostatin propeptide) and Becker, Gaucher disease (glucocerebrosidase), Hurler's disease (a-L-iduronidase), adenosine deaminase deficiency (adenosine deaminase), glycogen storage diseases (e.g., Fabry disease [a-galactosidase] and Pompe disease [lysosomal acid alpha-glucosidase]) and other metabolic disorders, congenital emphysema (alpha-1-antitrypsin), Lesch-Nyhan Syndrome (hypoxan thine guanine phosphoribosyl transferase), Niemann-Pick disease (sphingomyelinase), Tay-Sachs disease (lysosomal hexosaminidase A), Maple Syrup Urine Disease (branched-chain keto acid dehydrogenase), retinal degenerative diseases (and other diseases of the eye and retina; e.g., PDGF for macular degeneration and/or vasohibin or other inhibitors of VEGF or other angiogenesis inhibitors to treat/prevent retinal disorders, e.g., in Type I diabetes), diseases of solid organs such as brain (including Parkinson's Disease [GDNF], astrocytomas [endostatin, angiostatin and/or RNAi against VEGF], glioblastomas [endostatin, angiostatin and/or RNAi against VEGF]), liver, kidney, heart including congestive heart failure or peripheral artery disease (PAD) (e.g., by delivering protein phosphatase inhibitor I (I-1) and fragments thereof (e.g., IlC), serca2a, zinc finger proteins that regulate the phospholamban gene, Barkct, [32-adrenergic receptor, 2-adrenergic receptor kinase (BARK), phosphoinositide-3 kinase (PI3 kinase), S100A1, parvalbumin, adenylyl cyclase type 6, a molecule that effects G-protein coupled receptor kinase type 2 knockdown such as a truncated constitutively active bARKct; calsarcin, RNAi against phospholamban; phospholamban inhibitory or dominant-negative molecules such as phospholamban S 16E, etc.), arthritis (insulin-like growth factors), joint disorders (insulin-like growth factor 1 and/or 2), intimal hyperplasia (e.g., by delivering enos, inos), improve survival of heart transplants (superoxide dismutase), AIDS (soluble CD4), muscle wasting (insulin-like growth factor I), kidney deficiency (erythropoietin), anemia (erythropoietin), arthritis (anti-inflammatory factors such as I RAP and TNFa soluble receptor), hepatitis (a-interferon), LDL receptor deficiency (LDL receptor), hyperammonemia (ornithine transcarbamylase), Krabbe's disease (galactocerebrosidase), Batten's disease, spinal cerebral ataxias including SCA1, SCA2 and SCA3, phenylketonuria (phenylalanine hydroxylase), autoimmune diseases, and the like. The disclosure can further be used following organ transplantation to increase the success of the transplant and/or to reduce the negative side effects of organ transplantation or adjunct therapies (e.g., by administering immunosuppressant agents or inhibitory nucleic acids to block cytokine production). As another example, bone morphogenic proteins (including BNP 2, 7, etc., RANKL and/or VEGF) can be administered with a bone allograft, for example, following a break or surgical removal in a cancer patient.

In some embodiments, the virus vectors of the present disclosure can be employed to deliver a heterologous nucleic acid encoding a polypeptide or functional RNA to treat and/or prevent a liver disease or disorder. The liver disease or disorder may be, for example, primary biliary cirrhosis, nonalcoholic fatty liver disease (NAFLD), non-alcoholic steatohepatitis (NASH), autoimmune hepatitis, hepatitis B, hepatitis C, alcoholic liver disease, fibrosis, jaundice, primary sclerosing cholangitis (PSC), Budd-Chiari syndrome, hemochromatosis, Wilson's disease, alcoholic fibrosis, non-alcoholic fibrosis, liver steatosis, Gilbert's syndrome, biliary atresia, alpha-1-antitrypsin deficiency, alagille syndrome, progressive familial intrahepatic cholestasis, Hemophilia B, Hereditary Angioedema (HAE), Homozygous Familial Hypercholesterolemia (HoFH), Heterozygous Familial Hypercholesterolemia (HeFH), Von Gierke's Disease (GSD I), Hemophilia A, Methylmalonic Acidemia, Propionic Acidemia, Homocystinuria, Phenylketonuria (PKU), Tyrosinemia Type 1, Arginase 1 Deficiency, Argininosuccinate Lyase Deficiency, Carbamoyl-phosphate synthetase 1 deficiency, Citrullinemia Type 1, Citrin Deficiency, Crigler-Najjar Syndrome Type 1, Cystinosis, Fabry Disease, Glycogen Storage Disease 1b, LPL Deficiency, N-Acetylglutamate Synthetase Deficiency, Ornithine Transcarbamylase Deficiency, Ornithine Translocase Deficiency, Primary Hyperoxaluria Type 1, or ADA SCID.

The virus vectors described herein can also be used to produce induced pluripotent stem cells (iPS). For example, a virus vector of the disclosure can be used to deliver stem cell associated nucleic acid(s) into a non-pluripotent cell, such as adult fibroblasts, skin cells, liver cells, renal cells, adipose cells, cardiac cells, neural cells, epithelial cells, endothelial cells, and the like.

Nucleic acids encoding factors associated with stem cells are known in the art. Nonlimiting examples of such factors associated with stem cells and pluripotency include Oct-3/4, the SOX family (e.g., SOX 1, SOX2, SOX3 and/or SOX 15), the Klf family (e.g., Klfl, KHZ Klf4 and/or Klf5), the Myc family (e.g., C-myc, L-myc and/or N-myc), NANOG and/or LIN28.

In some embodiments, the modified vectors disclosed herein can be used to treat a lysosomal storage disorder such as a mucopolysaccharidosis disorder (e.g., Sly syndrome [β-glucuronidase], Hurler Syndrome [alpha-L-iduronidase], Scheie Syndrome [alpha-L-iduronidase], Hurler-Scheie Syndrome [alpha-L-iduronidase], Hunter's Syndrome [iduronate sulfatase], Sanfilippo Syndrome A [heparan sulfamidase], B [N-acetylglucosaminidase], C [acetyl-CoA:alpha-glucosaminide acetyltransferase], D [N-acetylglucosamine 6-sulfatase], Morquio Syndrome A [galactose-6-sulfate sulfatase], B [β-galactosidase], Maroteaux-Lamy Syndrome [N-acetylgalactosamine-4-sulfatase], etc.), Fabry disease (a-galactosidase), Gaucher's disease (glucocerebrosidase), or a glycogen storage disorder (e.g., Pompe disease; lysosomal acid alpha-glucosidase) as described herein. In some embodiments, the disclosure can also be practiced to treat and/or prevent a metabolic disorder such as diabetes (e.g., insulin), hemophilia (e.g., Factor IX or Factor VIII), a lysosomal storage disorder such as a mucopolysaccharidosis disorder (e.g., Sly syndrome [β-glucuronidase], Hurler Syndrome [alpha-L-iduronidase], Scheie Syndrome [alpha-L-iduronidase], Hurler-Scheie Syndrome [alpha-L-iduronidase], Hunter's Syndrome [iduronate sulfatase], Sanfilippo Syndrome A [heparan sulfamidase], B [N-acetylglucosaminidase], C [acetyl-CoA:alpha-glucosaminide acetyltransferase], D [N-acetylglucosamine 6-sulfatase], Morquio Syndrome A [galactoses-sulfate sulfatase], B [β-galactosidase], Maroteaux-Lamy Syndrome [N-acetylgalactosamine-4-sulfatase], etc.), Fabry disease (alpha-galactosidase), Gaucher's disease (glucocerebrosidase), or a glycogen storage disorder (e.g., Pompe disease; lysosomal acid alpha-glucosidase).

Gene transfer has substantial use for understanding and providing therapy for disease states. There are a number of inherited diseases in which defective genes are known and have been cloned. In general, the above disease states fall into two classes: deficiency states, usually of enzymes, which are generally inherited in a recessive manner, and unbalanced states, which may involve regulatory or structural proteins, and which are typically inherited in a dominant manner. For deficiency state diseases, gene transfer can be used to bring a normal gene into affected tissues for replacement therapy, as well as to create animal models for the disease using antisense mutations. For unbalanced disease states, gene transfer can be used to create a disease state in a model system, which can then be used in efforts to counteract the disease state. Thus, virus vectors according to the present disclosure permit the treatment and/or prevention of genetic diseases.

The virus vectors according to the present disclosure may also be employed to provide a functional RNA to a cell in vitro or in vivo. The functional RNA may be, for example, a non-coding RNA. In some embodiments, expression of the functional RNA in the cell can diminish expression of a particular target protein by the cell. Accordingly, functional RNA can be administered to decrease expression of a particular protein in a subject in need thereof. In some embodiments, expression of the functional RNA in the cell can increase expression of a particular target protein by the cell. Accordingly, functional RNA can be administered to increase expression of a particular protein in a subject in need thereof. In some embodiments, expression of the functional RNA can regulate splicing of a particular target RNA in a cell. Accordingly, functional RNA can be administered to regulate splicing of a particular RNA in a subject in need thereof. In some embodiments, expression of the functional RNA in the cell can regulate the function of a particular target protein by the cell. Accordingly, functional RNA can be administered to regulate the function of a particular protein in a subject in need thereof. Functional RNA can also be administered to cells in vitro to regulate gene expression and/or cell physiology, e.g., to optimize cell or tissue culture systems or in screening methods.

In some embodiments, the virus vectors disclosed herein may be contacted with a cell ex vivo. In some embodiments, the cell is a T-cell, such as an activated T-cell. In some embodiments, the cells (e.g. activated T-cells) are obtained from a subject, such as a human patient. In some embodiments, the cell upon contact with the virus vector is administered to a subject in need thereof.

In some embodiments, the virus vector comprises a heterologous nucleic acid encoding a chimeric antigen receptor (CAR). Thus, in some embodiments, the contacting of the virus vector with the T-cell results in the expression of the chimeric antigen receptor (CAR) to generate CAR T-cells. Therefore, in some embodiments, the disclosure provides methods of preparing CAR T-cells comprising contacting any one of the virus vectors disclosed herein with a T-cell ex vivo. The disclosure further provides CAR T-cells produced using any one of the methods disclosed herein, and methods of treating a subject in need thereof comprising administering to the subject the CAR T-cells disclosed herein. In some embodiments, the CAR T-cells are produced using T-cells obtained from the same subject (autologous T-cells), while in other embodiments, the CAR T-cells are produced using T-cells obtained from a healthy donor subject (allogenic T-cells). The subject in need of CAR T-cell administration may be identified by a doctor or a skilled medical practitioner, and may have any disease, such as cancer, for instance, acute lymphoblastic leukemia (ALL), diffuse large B-cell lymphoma (DLBCL), Hodgkin's lymphoma, acute myeloid leukemia (AML), or multiple myeloma.

T-cell exhaustion is a state of T-cell dysfunction that arises during many chronic infections and cancer, and has also been shown to reduce the effectiveness of CAR-T therapies. In some embodiments, the recombinant virus vectors disclosed herein are used in gene therapy methods (e.g. CAR-T therapy methods) for preventing, limiting, and/or reversing T-cell exhaustion. Therefore, the disclosure provides methods of alleviating, preventing, limiting, and/or reversing T-cell exhaustion in a subject, comprising administering to the subject an effective amount of any one of the viral vectors (e.g. AAV vectors), any one of the viral particles (e.g. AAV particles), and/or any one of the compositions disclosed herein.

In some embodiments, the virus vector comprises a heterologous nucleic acid that encodes an immunogen, such as an immunogenic polypeptide. Thus, in some embodiments, the contacting of the virus vector with the cell results in the expression of the immunogen. In some embodiments, the cell may be administered to a subject, and therefore, result in inducing an immune response in the subject against the immunogen. In some embodiments, a protective immune response is elicited. In some embodiments, the cell is an antigen-presenting cell (e.g., a dendritic cell). In some embodiments, the cells have been removed from a subject, the virus vector is introduced therein, and the cells are then administered back into the subject. Methods of removing cells from subject for manipulation ex vivo, followed by introduction back into the subject are known in the art (see, e.g., U.S. Pat. No. 5,399,346). Alternatively, the recombinant virus vector can be introduced into cells from a donor subject, into cultured cells, or into cells from any other suitable source, and the cells are administered to a subject in need thereof (i.e., a “recipient” subject).

In some embodiments, cells may be removed from a subject with cancer and contacted with a virus vector expressing a cancer cell antigen according to the instant disclosure. The modified cell is then administered to the subject, whereby an immune response against the cancer cell antigen is elicited. This method can be advantageously employed with immunocompromised subjects that cannot mount a sufficient immune response in vivo (i.e., cannot produce enhancing antibodies in sufficient quantities). Alternatively, the cancer antigen can be expressed as part of the virus capsid or be otherwise associated with the virus capsid (e.g., as described above). As another alternative, any other therapeutic nucleic acid (e.g., RNAi) or polypeptide (e.g., cytokine) known in the art can be administered to treat and/or prevent cancer.

It is known in the art that immune responses may be enhanced by immunomodulatory cytokines (e.g., alpha-interferon, beta-interferon, gamma-interferon, omega-interferon, tau-interferon, interleukin-1-alpha, interleukin-1β, interleukin-2, interleukin-3, interleukin-4, interleukin 5, interleukin-6, interleukin-7, interleukin-8, interleukin-9, interleukin-10, interleukin-11, interleukin-12, interleukin-13, interleukin-14, interleukin-18, B cell Growth factor, CD40 Ligand, tumor necrosis factor-alpha, tumor necrosis factor-β, monocyte chemoattractant protein-1, granulocyte-macrophage colony stimulating factor, and lymphotoxin). Accordingly, immunomodulatory cytokines (preferably, CTL inductive cytokines) may be administered to a subject in conjunction with the virus vector. Cytokines may be administered by any method known in the art. Exogenous cytokines may be administered to the subject, or alternatively, a nucleic acid encoding a cytokine may be delivered to the subject using a suitable vector, and the cytokine produced in vivo.

In addition, virus vectors according to the instant disclosure find use in diagnostic and screening methods, whereby a nucleic acid of interest is transiently or stably expressed in a cell culture system, or alternatively, a transgenic animal model.

The virus vectors of the present disclosure can also be used for various non-therapeutic purposes, including but not limited to use in protocols to assess gene targeting, clearance, transcription, translation, etc., as would be apparent to one skilled in the art. The virus vectors can also be used for the purpose of evaluating safety (spread, toxicity, immunogenicity, etc.) Such data, for example, are considered by the United States Food and Drug Administration as part of the regulatory approval process prior to evaluation of clinical efficacy.

In some embodiments, the modified virus capsids of the disclosure find use in raising antibodies against the novel capsid structures. In some embodiments, an exogenous amino acid sequence may be inserted into the modified virus capsid for antigen presentation to a cell, e.g., for administration to a subject to produce an immune response to the exogenous amino acid sequence.

In some embodiments, the virus capsids can be administered to block certain cellular sites prior to and/or concurrently with (e.g., within minutes or hours of each other) administration of a virus vector delivering a nucleic acid encoding a polypeptide or functional RNA of interest. For example, the inventive capsids can be delivered to block cellular receptors on liver cells and a delivery vector can be administered subsequently or concurrently, which may reduce transduction of liver cells, and enhance transduction of other targets (e.g., skeletal, cardiac and/or diaphragm muscle).

### Dosage and Modes of Administration

The virus vector may be introduced into the cells at the appropriate multiplicity of infection according to standard transduction methods suitable for the particular target cells. Titers of virus vector to administer can vary, depending upon the target cell type and number, and the particular virus vector, and can be determined by those of skill in the art without undue experimentation. In representative embodiments, at least about 10³ infectious units, optionally at least about 10⁵ infectious units are introduced to the cell.

The cell(s) into which the virus vector is introduced can be of any type, including but not limited to T-cells, neural cells (including cells of the peripheral and central nervous systems, in particular, brain cells such as neurons and oligodendrocytes), lung cells, cells of the eye (including retinal cells, retinal pigment epithelium, and corneal cells), epithelial cells (e.g., gut and respiratory epithelial cells), muscle cells (e.g., skeletal muscle cells, cardiac muscle cells, smooth muscle cells and/or diaphragm muscle cells), dendritic cells, pancreatic cells (including islet cells), hepatic cells, myocardial cells, bone cells (e.g., bone marrow stem cells), hematopoietic stem cells, spleen cells, keratinocytes, fibroblasts, endothelial cells, prostate cells, germ cells, and the like. In representative embodiments, the cell can be any progenitor cell. As a further possibility, the cell can be a stem cell (e.g., neural stem cell, liver stem cell). As still a further alternative, the cell can be a cancer or tumor cell. Moreover, the cell can be from any species of origin, as indicated above.

Suitable cells for ex vivo nucleic acid delivery are as described above. Dosages of the cells to administer to a subject will vary upon the age, condition and species of the subject, the type of cell, the nucleic acid being expressed by the cell, the mode of administration, and the like. Typically, at least about 10² to about 10⁸ cells or at least about 10³ to about 10⁶ cells will be administered per dose in a pharmaceutically acceptable carrier. In some embodiments, the cells transduced with the virus vector are administered to the subject in a therapeutically effective amount in combination with a pharmaceutical carrier.

In some embodiments, the virus vector is introduced into a cell and the cell can be administered to a subject to elicit an immunogenic response against the delivered polypeptide (e.g., expressed as a transgene or in the capsid). Typically, a quantity of cells expressing an immunogenically effective amount of the polypeptide in combination with a pharmaceutically acceptable carrier is administered. An “immunogenically effective amount” is an amount of the expressed polypeptide that is sufficient to evoke an active immune response against the polypeptide in the subject to which the pharmaceutical formulation is administered. In some embodiments, the dosage is sufficient to produce a protective immune response. The degree of protection conferred need not be complete or permanent, as long as the benefits of administering the immunogenic polypeptide outweigh any disadvantages thereof. Thus, the present disclosure provides a method of administering a nucleic acid to a cell, the method comprising contacting the cell with the virus vector, virus particle and/or composition of this disclosure.

Dosages of the virus vector and/or capsid to be administered to a subject depend upon the mode of administration, the disease or condition to be treated and/or prevented, the individual subject's condition, the particular virus vector or capsid, and the nucleic acid to be delivered, and the like, and can be determined in a routine manner. Exemplary doses for achieving therapeutic effects are titers of at least about 10⁵, about 10⁶, about 10⁷, about 10⁸, about 10⁹, about 10¹⁰, about 10¹¹, about 10¹², about 10¹³, about 10¹⁴, about 10¹⁵ transducing units, optionally about 10⁸-10¹³ transducing units. In some embodiments, more than one administration (e.g., two, three, four or more administrations) may be employed to achieve the desired level of gene expression over a period of various intervals, e.g., daily, weekly, monthly, yearly, etc.

Administration of the virus vectors, virus particles and/or capsids according to the present disclosure to a human subject or an animal in need thereof can be by any means known in the art. Optionally, the virus vectors, virus particles and/or compositions are delivered in a therapeutically effective dose in a pharmaceutically acceptable carrier. In some embodiments, a therapeutically effective amount of the virus vector, virus particle and/or capsid is delivered.

Exemplary modes of administration include oral, rectal, transmucosal, intranasal, inhalation (e.g., via an aerosol), buccal (e.g., sublingual), vaginal, intrathecal, intraocular, transdermal, in utero (or in ovo), parenteral (e.g., intravenous, subcutaneous, intradermal, intramuscular [including administration to skeletal, diaphragm and/or cardiac muscle], intradermal, intrapleural, intracerebral, and intraarticular), topical (e.g., to both skin and mucosal surfaces, including airway surfaces, and transdermal administration), intralymphatic, and the like, as well as direct tissue or organ injection (e.g., to liver, skeletal muscle, cardiac muscle, diaphragm muscle or brain). Administration can also be to a tumor (e.g., in or near a tumor or a lymph node). The most suitable route in any given case will depend on the nature and severity of the condition being treated and/or prevented and on the nature of the particular vector that is being used. The disclosure can also be practiced to produce noncoding RNA, such as antisense RNA, RNAi or other functional RNA (e.g., a ribozyme) for systemic delivery.

Injectables can be prepared in conventional forms, either as liquid solutions or suspensions, solid forms suitable for solution or suspension in liquid prior to injection, or as emulsions. Alternatively, one may administer the virus vector and/or virus capsids of the disclosure in a local rather than systemic manner, for example, in a depot or sustained-release formulation. Further, the virus vector and/or virus capsid can be delivered adhered to a surgically implantable matrix (e.g., as described in U.S. Patent Publication No. US-2004-0013645-A1).

### EXAMPLES

The following examples, which are included herein for illustration purposes only, are not intended to be limiting. As used herein, the terms STRD.201, STRD.202, STRD.203, STRD.204, STRD.205, STRD.206 and STRD. 207 are used to describe capsid protein sequences, and the terms, AAV-STRD. 201, AAV-STRD. 202, AAV-STRD.203 AAV-STRD.204 AAV-STRD.205, AAV-STRD.206, and AAV-STRD.207 are used to describe AAV vectors comprising the capsid proteins. However, the terms STRD.201, STRD.202, STRD.203, STRD.204, STRD.205, STRD.206 and STRD. 207 may be used in some contexts to describe AAV vectors comprising the named capsids, as will be apparent to the skilled artisan.

### Example 1: Evolution of AAV Capsid Protein Variants Comprising Transduction-Associated Peptides

An in vitro evolution process was used to prepare AAV capsid protein variants that, when incorporated into AAV vectors, provide enhanced transduction of the vectors into T-cells. The first step of this process involved identification of surface-exposed regions on the AAV capsid surface using cryo-electron microscopy. Selected residues within surface-exposed regions of the AAV capsid were then subjected to mutagenesis using degenerate primers with each codon substituted by nucleotides NNK and gene fragments combined together by Gibson assembly and/or multistep PCR. Here, amino acid residues 454-460 of SEQ ID NO: 1 were subjected to random mutagenesis to generate a library of recombinant capsid gene sequences. Each gene in this degenerate library was cloned into a wild type AAV genome to replace the original Cap-encoding DNA sequence, yielding a plasmid library. Plasmid libraries were then transfected into 293 producer cell lines with an adenoviral helper plasmid to generate AAV capsid libraries. Successful generation of AAV libraries was confirmed via DNA sequencing.

In order to identify the AAV vectors that can target and effectively transduce T-cells, the AAV libraries described above were subjected to multiple rounds of in vitro selection. Specifically, a first round of transduction into a mixed population of cells was performed, followed by two rounds of transduction into activated donor T-cells. At each stage, viral DNA was purified, PCR amplified and backcloned into AAV vectors, and used for the next round of selection. Further details of the general method used for combinatorial engineering and selection of AAV vectors is provided in WO 2019/195449, WO 2019/195423, and WO 2019/195444, the contents of each of which is incorporated herein by reference in its entirety. After three rounds of infection, AAV particles were isolated from the cultured T-cells. Specifically, cells were lysed and viral DNA was purified from the nuclear and cytosolic fractions of the T-cells, PCR amplified and backcloned into AAV vectors as described above.

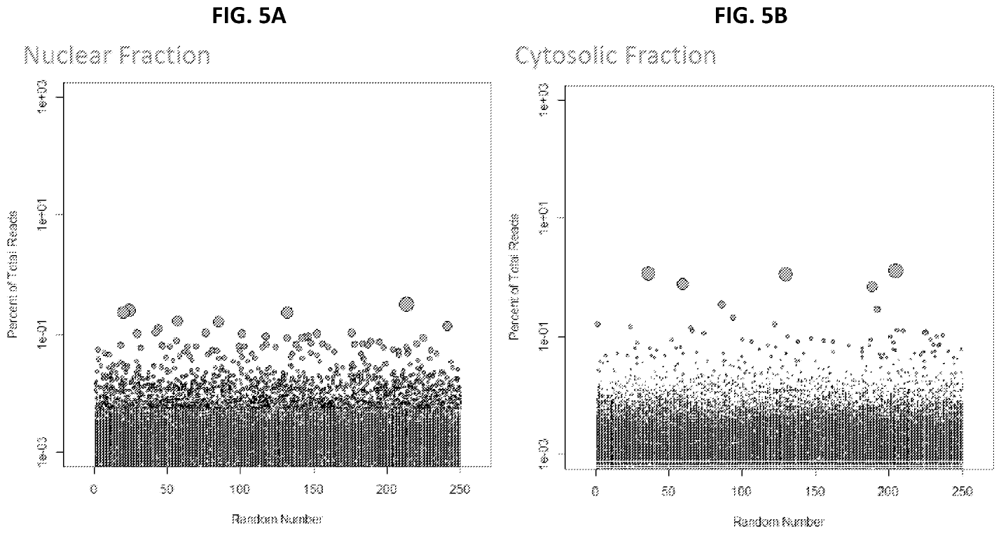

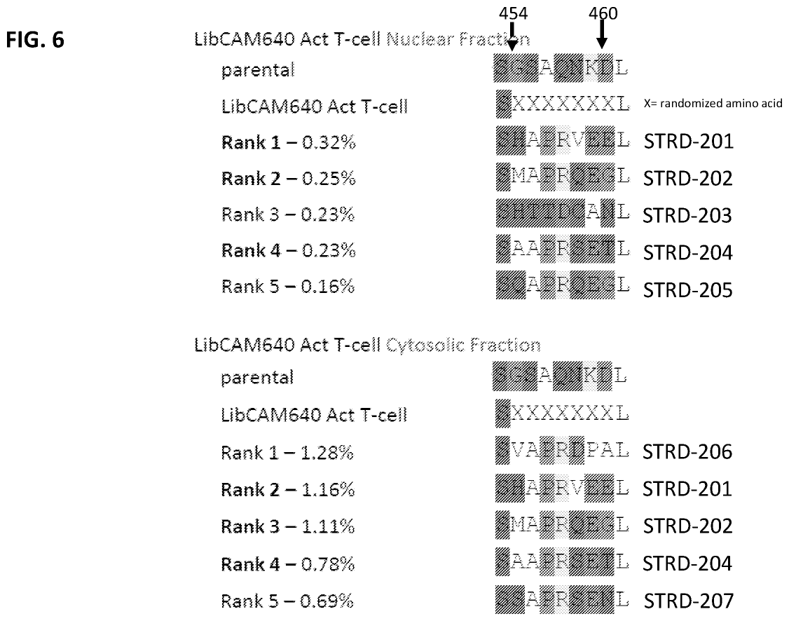

The AAV variants that were enriched in the nuclear and cytosolic fractions of the T-cells after the three rounds of selection and evolution described in Example 1 were sequenced to identify single AAV isolates. In the bubble plot shown in FIG. 5, the bubble size is proportional to the number of reads. The AAV variants that were most enriched in the nuclear fraction (AAV.STRD-203, 205), the cytosolic fraction (AAV.STRD-206, 207), or the nuclear and cytosolic fractions (AAV.STRD-201, 202 and 204) were sequenced to identify the amino acid residues present at amino acid positions 454-460. See FIG. 6 and Table 5. These results demonstrated that the recombinant AAV virions comprising variant capsid proteins comprising the transduction-associated peptides of Table 5 were able to effectively transduce T-cells.

TABLE 5
Transduction-associated peptides identified
using an in vitro evolution process

| Variant | | | SEQ ID NO of |
|---|---|---|---|
| AAV6 | Transduction | SEQ | corresponding |
| capsid | enhancing | ID | AAV6 |
| protein | peptide | NO: | capsid variant |
| STRD-201 | HAPRVEE | 17 | 2 |
| STRD-202 | MAPRQEG | 18 | 4 |
| STRD-203 | HTTDCAN | 19 | 6 |
| STRD-204 | AAPRSET | 20 | 8 |
| STRD-205 | QAPRQEG | 21 | 10 |
| STRD-206 | VAPRDPA | 22 | 12 |
| STRD-207 | SAPRSEN | 23 | 14 |

### Example 2: Manufacturability of AAV Vectors Comprising Transduction-Associated Peptides

To determine whether the various AAV vectors identified in Example 1 may be manufactured in large-scale systems, the AAVs were produced according to standard methods, and yield was compared to that of wild type AAV6 vector.

AAVs were produced in HEK293 cells according to a standard triple transfection protocol. Briefly, the cells were transfected with (i) a plasmid comprising either the wild type AAV9 capsid sequence, or the variant capsid sequences listed in Table 5; (ii) a plasmid comprising a 5′ITR, a transgene, and a 3′ ITR sequence; and (iii) a plasmid comprising helper genes necessary for AAV production. AAVs were purified from the supernatant of the cell culture. Subsequently, the yield of each AAV was measured using a PCR-based quantification approach.

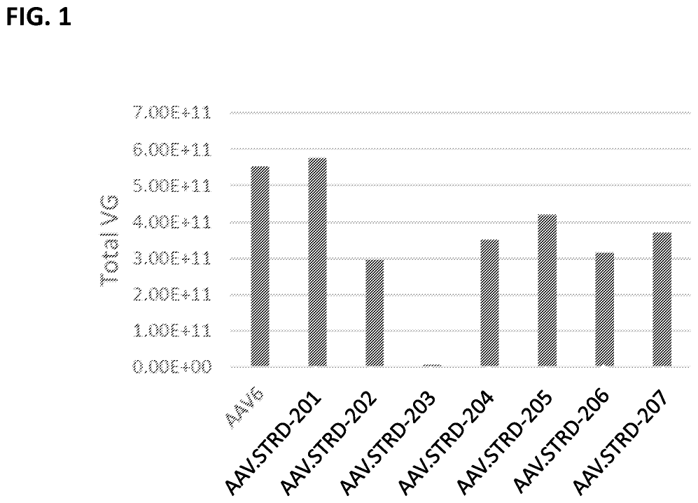

As shown in FIG. 1 and Table 6, recombinant AAV vectors comprising the capsid sequence of STRD-201 (termed “AAV.STRD-201” here) had a higher yield than the yield of wild type AAV6. Further, the yield of AAV. STRD-204, AAV.STRD-205, AAV. STRD-206 and AAV.STRD-207 was comparable to the yield of wild type AAV6.

These data confirm that the AAV vectors comprising the capsid variant proteins are suitable for commercial manufacturing.

TABLE 6

| Recombinant | Volume | Titer (vector | Yield |
|---|---|---|---|
| AAV | (mL) | genome (vg)/mL) | (total vg) |
| AAV6 | 2.80 | 1.97E+11 | 5.52E+11 |
| AAV.STRD-201 | 3.25 | 1.77E+11 | 5.75E+11 |
| AAV.STRD-202 | 2.25 | 1.31E+11 | 2.95E+11 |
| AAV.STRD-203 | 2.25 | 2.64E+09 | 5.94E+09 |
| AAV.STRD-204 | 2.50 | 1.40E+11 | 3.50E+11 |
| AAV.STRD-205 | 2.50 | 1.68E+11 | 4.20E+11 |
| AAV.STRD-206 | 2.50 | 1.26E+11 | 3.15E+11 |
| AAV.STRD-207 | 2.50 | 1.48E+11 | 3.70E+11 |

### Example 3: Characterizing the Expression of GFP Transgene by AAV Variants in T-Cells

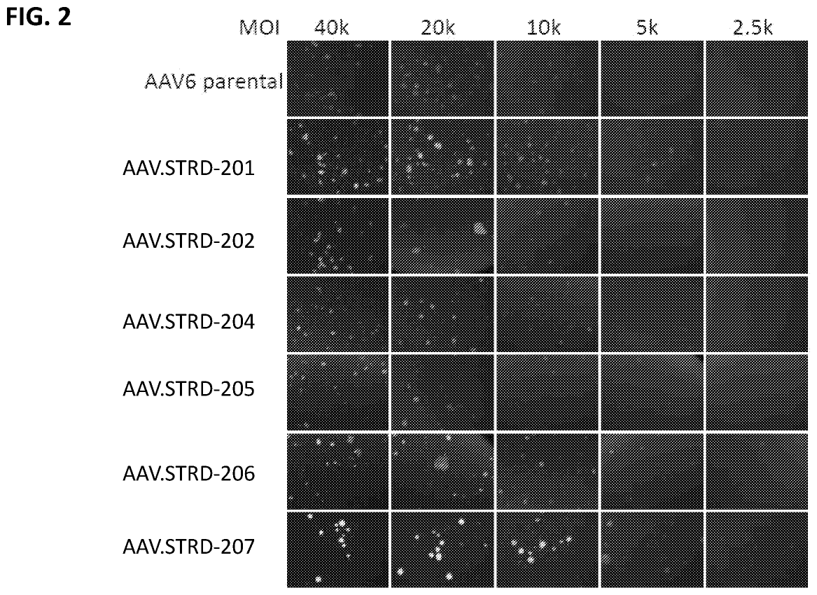

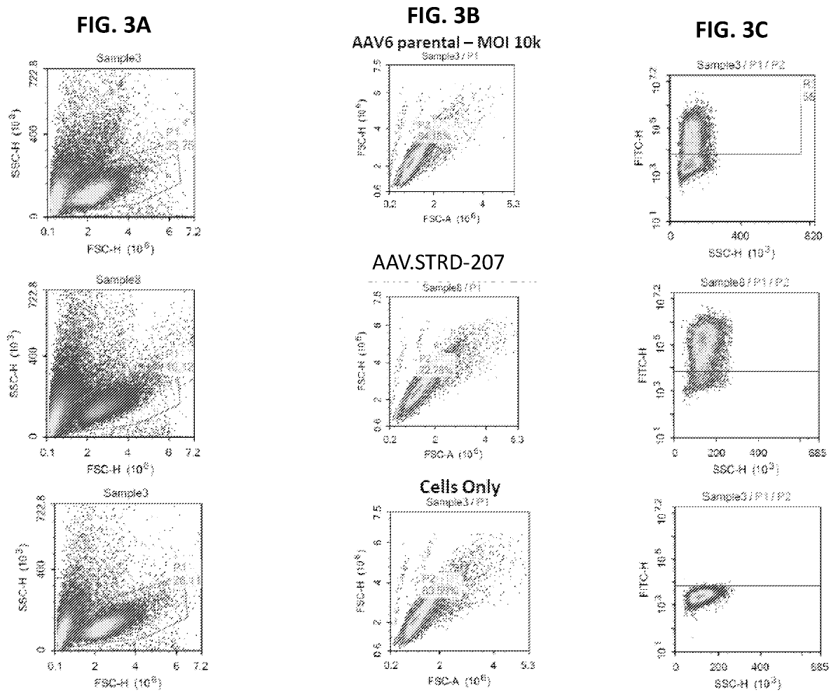

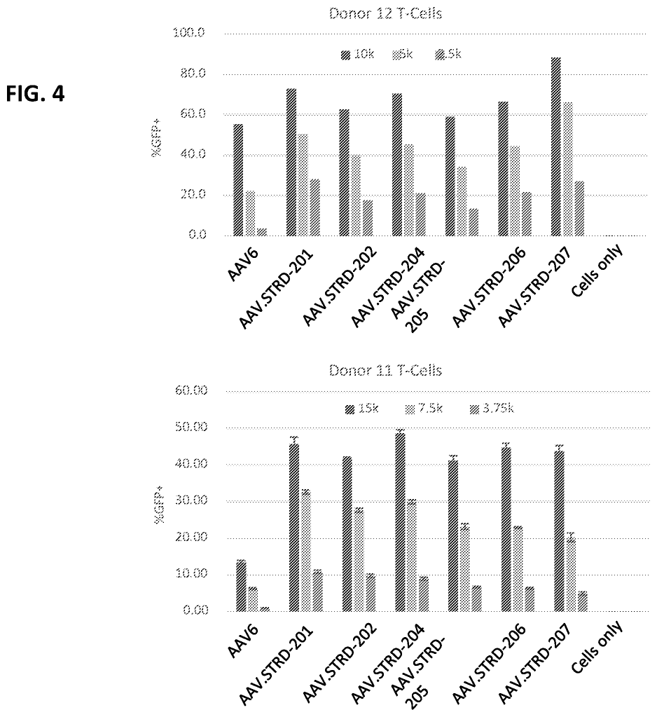

Recombinant AAV variants, AAV.STRD-201, AAV. STRD-202, AAV. STRD-204, AAV.STRD-205, AAV.STRD-206, and AAV.STRD-207 or the wild type AAV6 vector carrying a GFP transgene sequence were transduced into activated T-cells. Since T-cells clump during expansion, the cells were pipetted up and down or mixed prior to imaging. The expression of GFP was observed by microscopy and images from the experiment are shown in FIG. 2. Higher GFP expression indicates more efficient transduction of the viral vector to the T-cells. As seen from the images in FIG. 2, all the AAV variants show brighter green fluorescence signal and therefore, higher expression of GFP in activated T-cells, as compared to the wild type AAV6 viral vector. Among the recombinant AAV variants, AAV. STRD-201 and AAV. STRD-207 showed particularly enhanced GFP expression indicating more enhanced transduction into T-cells. To further analyze the level of GFP expression from the AAV variants in comparison to the wild type AAV6 viral vector, T-cells transduced with either AAV6 vector or AAV. STRD-207 variant were subjected to flow cytometry, with T-cells alone being used as a negative control. As shown in FIG. 3C, an increased proportion of cells transduced with the AAV.STRD-207 variant show higher GFP signal (indicated by the FITC signal above the blue line), as compared to the population that was transduced by the AAV6 parental vector. The GFP expression in cells transduced with AAV variants (AAV.STRD-201, AAV. STRD-202, AAV. STRD-204, AAV. STRD-205, AAV. STRD-206 and AAV. STRD-207) is further quantified in FIG. 4, which shows the % of GFP-positive cells in a given population (indicated by bars) as well as the mean intensity of GFP in that population (indicated by line graph). The results show that an increase in the number of GFP positive cells corresponds well with the increase in the mean intensity of the GFP signal in cells transduced by the AAV variants, as compared to the wild type AAV6, indicating that enhanced transduction of the AAV variants into T-cells results in the increased GFP expression in the T-cells.

The foregoing is illustrative of the present invention, and is not to be construed as limiting thereof. The invention is defined by the following claims, with equivalents of the claims to be included therein.

### Numbered Embodiments

The following list of embodiments is included herein for illustration purposes only and is not intended to be comprehensive or limiting. The subject matter to be claimed is expressly not limited to the following embodiments.

Embodiment 1. A recombinant adeno-associated virus (AAV) vector comprising a capsid protein, wherein the capsid protein comprises a transduction-associated peptide having the sequence of any one of SEQ ID NOs: 17 to 23.

Embodiment 2. The recombinant AAV vector of embodiment 1, wherein the capsid protein comprises an amino acid sequence that has at least 90%, at least 95%, at least 96%, at least 97%, at least 98%, or at least 99% identity to SEQ ID NO: 1.

Embodiment 3. The recombinant AAV vector of embodiment 1 or embodiment 2, wherein the transduction-associated peptide replaces the amino acids corresponding to amino acids 454-460 of SEQ ID NO: 1.

Embodiment 4. The recombinant AAV vector of embodiment 1, wherein the capsid protein comprises an amino acid sequence selected from the group consisting of SEQ ID NOs: 2, 4, 6, 8, 10, 12, and 14, or a sequence at least 90%, at least 95%, at least 96%, at least 97%, at least 98%, or at least 99% identical thereto.

Embodiment 5. A recombinant AAV vector comprising a capsid protein, wherein the capsid protein comprises the sequence of SEQ ID NO: 1, wherein amino acids 454-460 of SEQ ID NO: 1 are replaced by a transduction-associated peptide comprising the sequence X1-X2-X3-X4-X5-X6-X7 (SEQ ID NO: 24).

Embodiment 6. The recombinant AAV vector of embodiment 5, wherein X1 is not G, X2 is not S, X3 is not A, X4 is not Q, X5 is not N, X6 is not K, and/or X7 is not D.

Embodiment 7. The recombinant AAV vector of any one of embodiments 5-6, wherein X1 is H, M, A, Q, V, or S.

Embodiment 8. The recombinant AAV vector of any one of embodiments 5-7, wherein X2 is A or T.

Embodiment 9. The recombinant AAV vector of any one of embodiments 5-8, wherein X3 is P or T.

Embodiment 10. The recombinant AAV vector of any one of embodiments 5-9, wherein X4 is R or D.

Embodiment 11. The recombinant AAV vector of any one of embodiments 5-10, wherein X5 is V, Q, C, S, or D.

Embodiment 12. The recombinant AAV vector of any one of embodiments 5-11, wherein X6 is E, A, or P.

Embodiment 13. The recombinant AAV vector of any one of embodiments 5-12, wherein X7 is E, G, N, T, or A.

Embodiment 14. The recombinant AAV vector of embodiment 5, wherein X1 is H, X2 is A, X3 is P, X4 is R, X5 is V, X6 is E, and X7 is E.

Embodiment 15. The recombinant AAV vector of embodiment 5, wherein X1 is M, X2 is A, X3 is P, X4 is R, X5 is Q, X6 is E, and X7 is G.

Embodiment 16. The recombinant AAV vector embodiment 5, wherein X1 is H, X2 is T, X3 is T, X4 is D, X5 is C, X6 is A, and X7 is N.

Embodiment 17. The recombinant AAV vector of embodiment 5, wherein X1 is A, X2 is A, X3 is P, X4 is R, X5 is S, X6 is E, and X7 is T.

Embodiment 18. The recombinant AAV vector of embodiment 5, wherein X1 is Q, X2 is A, X3 is P, X4 is R, X5 is Q, X6 is E, and X7 is G.

Embodiment 19. The recombinant AAV vector of embodiment 5, wherein X1 is V, X2 is A, X3 is P, X4 is R, X5 is D, X6 is P, and X7 is A.

Embodiment 20. The recombinant AAV vector of embodiment 5, wherein X1 is S, X2 is A, X3 is P, X4 is R, X5 is S, X46 is E, and X7 is N.

Embodiment 21. The recombinant AAV vector of embodiment 5, wherein the capsid protein comprises an amino acid sequence having at least about 95%, at least about 96%, at least about 97%, at least about 98%, or at least about 99% identity to SEQ ID NO: 1.

Embodiment 22. The recombinant AAV vector of embodiment 21, wherein the capsid protein comprises an amino acid sequence having about 99% identity to SEQ ID NO: 1.

Embodiment 23. The recombinant AAV vector of embodiment 5, wherein the capsid protein comprises an amino acid sequence selected from the group consisting of SEQ ID NOs: 2, 4, 6, 8, 10, 12, and 14.

Embodiment 24. A recombinant AAV vector comprising a capsid protein, wherein the capsid protein comprises a transduction-associated peptide having an amino acid sequence of SEQ ID NO: 16, wherein the transduction-associated peptide replaces amino acids 454-460 relative to SEQ ID NO: 1.

Embodiment 25. The recombinant AAV vector of embodiment 24, wherein the transduction-associated peptide has an amino acid sequence of any one of SEQ ID NOs: 17-23.

Embodiment 26. A nucleic acid encoding a recombinant AAV capsid protein having the sequence of any one of SEQ ID NOs: 2, 4, 6, 8, 10, 12, and 14.

Embodiment 27. The nucleic acid of embodiment 26, wherein the nucleic acid comprises a sequence selected from the group consisting of SEQ ID NOs: 3, 5, 7, 9, 11, 13, and 15.

Embodiment 28. The nucleic acid of embodiment 26 or embodiment 27, wherein the nucleic acid is a DNA sequence.

Embodiment 29. The nucleic acid of embodiment 26 or embodiment 27, wherein the nucleic acid is an RNA sequence.

Embodiment 30. An expression vector comprising the nucleic acid of any one of embodiments 26-29.

Embodiment 31. A cell comprising the nucleic acid of any one of embodiments 26-29, or the expression vector of embodiment 30.

Embodiment 32. The recombinant AAV vector of any one of embodiments 1-25, further comprising a cargo nucleic acid encapsidated by the capsid protein.

Embodiment 33. The recombinant AAV vector of embodiment 32, wherein the cargo nucleic acid encodes a therapeutic protein or a therapeutic RNA.

Embodiment 34. The recombinant AAV vector of any one of embodiments 32 to 33, wherein the AAV vector exhibits increased transduction into a cell compared to an AAV vector that does not comprise the transduction-associated peptide.

Embodiment 35. The AAV vector of embodiment 34, wherein the cell is a T-cell.

Embodiment 36. The AAV vector of embodiment 35, wherein the AAV vector exhibits increased transduction into the nucleus of a T-cell as compared to an AAV vector that does not comprise the transduction-associated peptide.

Embodiment 37. The AAV vector of embodiment 35, wherein the AAV vector exhibits increased transduction into the cytosol of a T-cell as compared to an AAV vector that does not comprise the transduction-associated peptide.

Embodiment 38. A composition, comprising the recombinant AAV vector of any one of embodiments 1-25 or 32-37, the nucleic acid of any one of embodiments 26-29, the expression vector of embodiment 30, or the cell of embodiment 31.

Embodiment 39. A pharmaceutical composition, comprising the cell of embodiment 31 or the recombinant AAV vector of any one of embodiments 1-25 or 32-37; and a pharmaceutically acceptable carrier.

Embodiment 40. A method of delivering an AAV vector into a cell, comprising contacting the cell with the AAV vector of any one of embodiments 1-25 or 32-37.

Embodiment 41. The method of embodiment 40, wherein the contacting of the cell is performed in vitro, ex vivo or in vivo.

Embodiment 42. The method of embodiment 40 or embodiment 41, wherein the cell is a T-cell.

Embodiment 43. A method of treating a subject in need thereof, comprising administering to the subject an effective amount of an AAV vector of any one of embodiments 1-25 or 32-37.

Embodiment 44. A method of treating a subject in need thereof, comprising administering to the subject a cell that has been contacted ex vivo with an AAV vector of any one of embodiments 1-25 or 32-37.

Embodiment 45. The method of embodiment 43 or embodiment 44, wherein the subject is a mammal.

Embodiment 46. The method of embodiment 45, wherein the subject is a human.

Embodiment 47. An AAV vector of any one of embodiments 1-25 or 32-37 for use as a medicament.

Embodiment 48. An AAV vector of any one of embodiments 1-25 or 32-37 for use in a method of treatment of a subject in need thereof.

1. A recombinant adeno-associated virus (AAV) vector comprising a capsid protein, wherein the capsid protein comprises a peptide comprising the sequence of any one of SEQ ID NOs: 17 to 23.

2. (canceled)

3. The recombinant AAV vector of claim 1, wherein the capsid protein comprises an amino acid sequence that has at least 95% sequence identity to SEQ ID NO: 1 and wherein the peptide replaces the amino acids corresponding to amino acids 454-460 of SEQ ID NO: 1.

4. (canceled)

5. A recombinant AAV vector comprising a capsid protein, wherein the capsid protein comprises the sequence of SEQ ID NO: 1, wherein amino acids 454-460 of SEQ ID NO: 1 are replaced by a peptide comprising the sequence X1-X2-X3-X4-X5-X6-X7 (SEQ ID NO: 24), wherein X1 is not G, X2 is not S, X3 is not A, X4 is not Q, X5 is not N, X6 is not K, and/or X7 is not D.

6. (canceled)

7. The recombinant AAV vector of claim 5, wherein:
X1 is H, M, A, Q, V, or S;
X2 is A or T;
X3 is P or T;
X4 is R or D;
X5 is V, Q, C, S, or D;
X6 is E, A, or P; and
X7 is E, G, N, T, or A.

8.-22. (canceled)

23. A recombinant AAV vector comprising a capsid protein, wherein the capsid protein comprises an amino acid sequence selected from the group consisting of SEQ ID NOs: 2, 4, 6, 8, 10, 12, and 14.

24.-25. (canceled)

26. A nucleic acid encoding a recombinant AAV capsid protein of claim 23.

27. The nucleic acid of claim 26, wherein the nucleic acid comprises a sequence selected from the group consisting of SEQ ID NOs: 3, 5, 7, 9, 11, 13, and 15.

28. The nucleic acid of claim 26, wherein the nucleic acid is a DNA sequence or an RNA sequence.

29. (canceled)

30. An expression vector comprising the nucleic acid of claim 26.

31. A cell comprising the nucleic acid of claim 26.

32. The recombinant AAV vector of claim 1, further comprising a cargo nucleic acid encapsidated by the capsid protein.

33. The recombinant AAV vector of claim 32, wherein the cargo nucleic acid encodes a therapeutic protein or a therapeutic RNA.

34. The recombinant AAV vector of claim 32, wherein the AAV vector exhibits increased transduction into a cell compared to an AAV vector that does not comprise the transduction-associated peptide.

35. The AAV vector of claim 34, wherein the cell is a T-cell.

36. The AAV vector of claim 35, wherein the AAV vector exhibits increased transduction into the nucleus or cytosol of a T-cell as compared to an AAV vector that does not comprise the peptide.

37.-38. (canceled)

39. A pharmaceutical composition, comprising the cell of claim 31 and a pharmaceutically acceptable carrier.

40. A method of delivering an AAV vector into a cell, comprising contacting the cell with the AAV vector of claim 1.

41. (canceled)

42. The method of claim 40, wherein the cell is a T-cell.

43. A method of treating a subject in need thereof, comprising administering to the subject an effective amount of an AAV vector of claim 1.

44. A method of treating a subject in need thereof, comprising administering to the subject a cell that has been contacted ex vivo with an AAV vector of claim 1.

45.-48. (canceled)
